(19) United States
(12) Patent Application Publication
(10) Pub. No.: US 2026/0355171 A1
(43) Pub. Date: Nov. 12, 2026

(54) INHIBITORS AND USES THEREFOR

(71) Applicant: Macquarie University, North Ryde (AU)

(72) Inventors: Lars Matthias Ittner, North Ryde (AU); Daryl Ariawan, North Ryde (AU); Ole Tietz, North Ryde (AU); Janet Van Eersel, North Ryde (AU)

(21) Appl. No.: 19/101,538

(22) PCT Filed: Aug. 7, 2023
(86) PCT No.: PCT/AU2023/050736

§ 371 Date: Feb. 5, 2025

(30) Foreign Application Priority Data
Aug. 5, 2022 (AU) 2022902202

(51) Int. Cl. A61K 31/4545; A61K 31/517; A61P 31/12; C07D 401/14; C07D 471/04

(52) U.S. Cl. CPC A61K 31/4545; A61K 31/517; A61P 31/12; C07D 401/14; C07D 471/04

(57) ABSTRACT

Disclosed are compounds of Formula I and their use as inhibitors of the viral protease non-structural protein 5 (NSP5). Typically, the NSP5 is derived from a coronavirus, more typically from SARS-CoV-2. More particularly, the invention relates to compounds of Formula (I) and their use for treating or inhibiting the development of a condition in which inhibiting NSP5 activity is associated with effective treatment or inhibition, including a coronavirus infection, for treating or inhibiting the development of a condition associated with a coronavirus infection, for treating or inhibiting the development of neurological symptoms of, and/or neurological complications associated with, a coronavirus infection and for treating or inhibiting the development or progression of long COVID following SARS-CoV-2 infection.

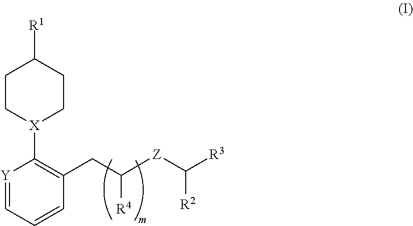

### FIELD OF THE INVENTION

This application claims priority to Australian Provisional Patent Application No. 2022902202 entitled “Inhibitors and Uses Therefor” filed 5 Aug. 2022, the contents of which are incorporated herein by reference in their entirety.

This invention relates generally to compounds of Formula I and their use as inhibitors of the viral protease non-structural protein 5 (NSP5). Typically, the NSP5 is derived from a coronavirus, more typically from SARS-CoV-2. More particularly, the invention relates to compounds of Formula I and their use for treating or inhibiting the development of a condition in which inhibiting NSP5 activity is associated with effective treatment or inhibition, including a coronavirus infection, for treating or inhibiting the development of a condition associated with a coronavirus infection, for treating or inhibiting the development of neurological symptoms of, and/or neurological complications associated with, a coronavirus infection and for treating or inhibiting the development or progression of long COVID following SARS-CoV-2 infection.

### BACKGROUND OF THE INVENTION

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavor to which this specification relates.

Viral infections cause a significant economic and social burden to be placed on society. This has been particularly demonstrated by the recent pandemic caused by severe acute respiratory syndrome coronavirus 2 (SARS-CoV-2) which emerged in China in 2019. Extremely infectious, SARS-CoV-2 has resulted in substantial mortality and morbidity worldwide. The clinical spectrum of disease (COVID-19) caused by SARS-CoV-2 varies from asymptomatic to mild or moderate disease to severe clinical manifestations characterised by respiratory failure (acute respiratory distress syndrome), acute lung inflammation, sepsis, septic shock and multiple organ dysfunction syndrome. Increasingly, neurological symptoms are associated with COVID-19, both in the acute phase and during recovery from COVID-19.

“Long COVID” (also referred to as post COVID syndrome) is a term used to describe the presence of persistent (or new) symptoms weeks, months or even years after an individual has acquired a SARS-CoV-2 infection, irrespective of viral status. Long COVID can be continuous or relapsing and remitting in nature. Common symptoms of long COVID include fatigue, heart symptoms or conditions (such as myocarditis), digestive symptoms including diarrhea and stomach pain, joint or muscle pain, and a range of neurological symptoms.

Common neurological impairments associated with the acute phase of a SARS-CoV-2 infection include headaches, fatigue, impaired or loss of smell (anosmia) and impaired or loss of taste (dysgeusia). Longer term neurological effects of SARS-CoV-2 infection are increasingly being reported in those experiencing long COVID, including fatigue, dizziness, difficulty sleeping, anxiety, depression, and cognitive challenges and/or deficits such as loss of memory, impaired concentration, poor attention, confusion and difficulties with thinking and decision making.

The current pandemic caused by SARS-CoV-2 is a global health emergency that requires the development not only of new vaccines in a bid to inhibit the spread of viral infection but also new therapeutics to treat infections caused by the virus and to treat or lessen the impact of COVID-19 symptoms and conditions associated with the disease.

Coronaviruses, including SARS-CoV-2, have a genome containing at least six open reading frames. The major open reading frame encodes two overlapping polyproteins that are cleaved by two proteases, non-structural protein 3 or NSP3 (also known as papain-like protease or PLPro) and non-structural protein 5 or NSP5 (also known as main protease, Mpro, 3C-like protease or 3CLpro), into 16 non-structural proteins, NSP1-16, which are required for viral replication and maturation. These non-structural proteins are essential for the assembly of the viral replication transcription complex.

NSP5 is a cysteine protease and is responsible for cleavage of viral polyproteins. Its function and sequence are highly conserved amongst coronaviruses, with SARS-CoV-2 NSP5 displaying approximately 98% sequence identity with SARS-CoV NSP5. Importantly, the greatest degree of sequence conservation is around the active site. In addition to its importance for viral replication, NSP5 has been shown to play a role in viral entry into host cells and, more recently, has been found to cleave host proteins, impacting on host cell function. NSP5 has a substrate specificity that is distinct from the majority of human proteases, thereby making it a viable therapeutic target.

Due to its important role in viral replication, host cell entry and host cell damage, NSP5 is a potential target for therapies for coronavirus infections. Accordingly, compounds which inhibit NSP5 activity are desired.

### SUMMARY OF THE INVENTION

The present invention is predicated in part on the discovery of particular compounds that inhibit non-structural protein 5 (NSP5), in particular SARS-CoV-2 NSP5. Accordingly, the inventors have conceived that such compounds will be useful for: treating and inhibiting the development of conditions associated with NSP5 activity, including a coronavirus infection; treating or inhibiting the development of conditions associated with coronavirus infections; treating or inhibiting the development of neurological symptoms of, and/or neurological complications or impairments associated with, a coronavirus infection; and treating or inhibiting the development or progression of long COVID following SARS-CoV-2 infection.

In one aspect, there is provided a compound of Formula I:

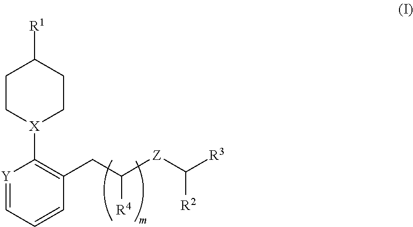

or a pharmaceutically acceptable salt thereof,
wherein:
R¹ is selected from the group consisting of H, hydroxy, optionally substituted C₁₋₆ alkyl, —NH₂, —C(O)—NH₂, optionally substituted —C(O)—C₁₋₆ alkyl, halogen, —C(OH)—NH₂, —C(O)—OH, —CH₂—NH₂, —C(O)H and optionally substituted C₁₋₆ alkoxy;
R² is selected from the group consisting of H, ═O, hydroxy, halogen, optionally substituted C₁₋₆ alkyl and optionally substituted C₁₋₆ alkoxy; and
R³ is selected from the group consisting of optionally substituted C₆₋₁₈ aryl, optionally substituted 5- to 18-membered heteroaryl, optionally substituted C₅₋₁₈ cycloalkyl, optionally substituted 5- to 18-membered heterocycloalkyl, or —C₁₋₆ alkylene-R⁵; or
R² and R³ together with the carbon atom to which they are attached form an optionally substituted C₆₋₁₈ aryl, optionally substituted 5- to 18-membered heteroaryl, optionally substituted C₅₋₁₈ cycloalkyl and optionally substituted 5- to 18-membered heterocycloalkyl;
R⁴ is selected from the group consisting of H, ═O, hydroxy, halogen, optionally substituted C₁₋₆ alkyl and optionally substituted C₁₋₆ alkoxy;
R⁵ is selected from the group consisting of optionally substituted C₆₋₁₈ aryl, optionally substituted 5- to 18-membered heteroaryl, optionally substituted C₅₋₁₈ cycloalkyl and optionally substituted 5- to 18-membered heterocycloalkyl;
m is 0 or 1;
X is selected from the group consisting of C and N;
Y is selected from the group consisting of C and N; and
Z is selected from the group consisting of C, O, N and S.

In some embodiments, the compound is other than a compound of Formula IV, V, VII, VIII, IX, XI and/or XII:

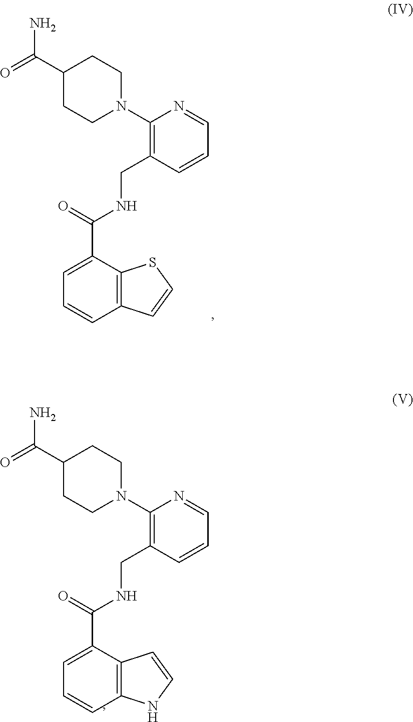 [SMILES: NC(=O)C1CCN(c2ncccc2CNC(=O)c2cccc3ccsc23)CC1 ; NC(=O)C1CCN(c2ncccc2CNC(=O)c2cccc3[nH]ccc23)CC1]

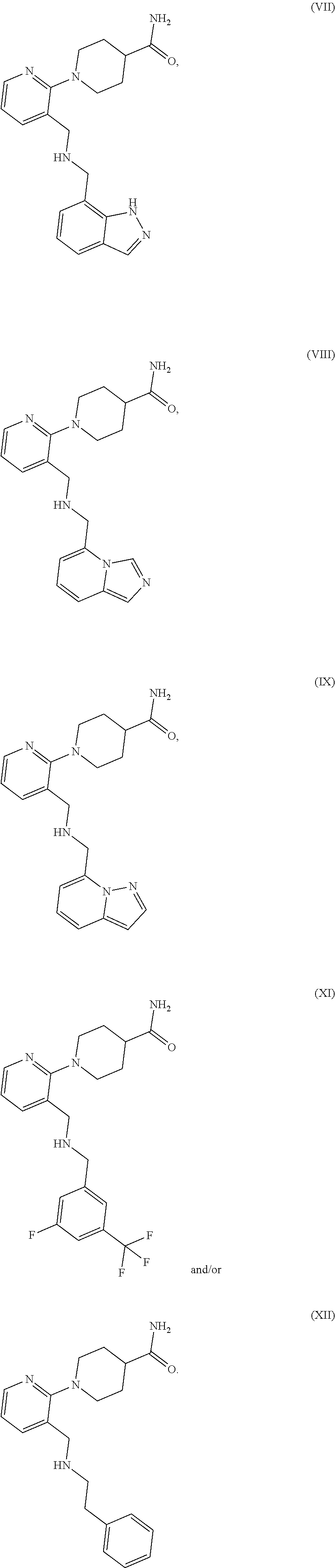 [SMILES: NC(=O)C1CCN(c2ncccc2CNCc2cccc3cn[nH]c23)CC1 ; NC(=O)C1CCN(c2ncccc2CNCc2cccc3cncn23)CC1 ; NC(=O)C1CCN(c2ncccc2CNCc2cccc3ccnn23)CC1 ; NC(=O)C1CCN(c2ncccc2CNCc2cc(F)cc(C(F)(F)F)c2)CC1 ; NC(=O)C1CCN(c2ncccc2CNCCc2ccccc2)CC1]

In some embodiments, R¹ is selected from the group consisting of —C(O)—NH₂, optionally substituted —C(O)—C₁₋₆ alkyl, —C(OH)—NH₂ and —C(O)—OH; especially —C(O)—NH₂.

In some embodiments, R² is H or ═O.

In some embodiments, R³ is selected from the group consisting of optionally substituted C₆₋₁₈ aryl and optionally substituted 5- to 18-membered heteroaryl; especially optionally substituted 5- to 18-membered heteroaryl.

In some embodiments, R³ is:

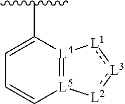

wherein L¹, L², L³, L⁴ and L⁵ are independently selected from the group consisting of C, O, N and S. In particular embodiments, R³ is:

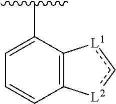

wherein L¹ and L² are independently selected from the group consisting of C, O, N and S; especially wherein L¹ is C and L² is N or L¹ is S and L² is C. In some embodiments, R³ is:

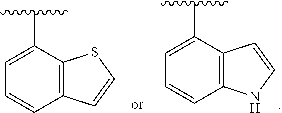 [SMILES: *c1cccc2ccsc12 ; *c1cccc2[nH]ccc12]

In alternative embodiments, R³ is selected from the group consisting of:

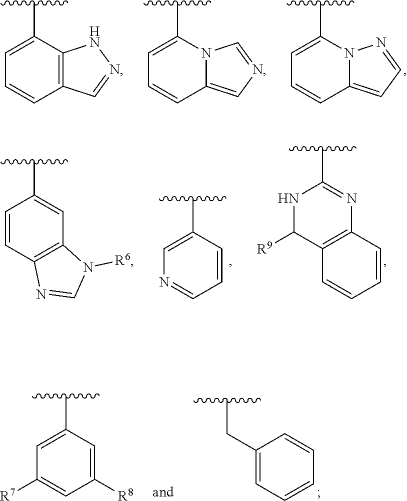

wherein R⁶ is selected from the group consisting of H, C₁₋₆ alkyl and halogen; R⁷ is selected from the group consisting of H, C₁₋₆ alkyl, halogen and C₁₋₆ haloalkyl; R⁸ is selected from the group consisting of H, C₁₋₆ alkyl, halogen and C₁₋₆ haloalkyl; and R⁹ is selected from the group consisting of H, ═O, hydroxy, halogen and C₁₋₆ alkyl.

In some embodiments, R⁶ is —CH(CH₂)₂; R⁷ is —CF₃; R⁸ is F; and/or R⁹ is ═O.

In alternative embodiments, R² and R³ together with the carbon atom to which they are attached form an optionally substituted 5- to 18-membered heteroaryl, such as:

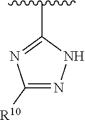

wherein R¹⁰ is selected from the group consisting of H, C₁₋₆ alkyl, C₃₋₆ cycloalkyl and halogen, and wherein

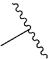

indicates the point of attachment to Z.

In particular embodiments, R¹⁰ is cyclopropyl.

In some embodiments, m is 0. In alternative embodiments, m is 1, and R⁴ is ═O.

In some embodiments, X is N.

In particular embodiments, Y is N.

In exemplary embodiments, Z is C or N; especially N.

In particular embodiments, the compound is a compound of any one of Formula VI, X and XIII-XVI:

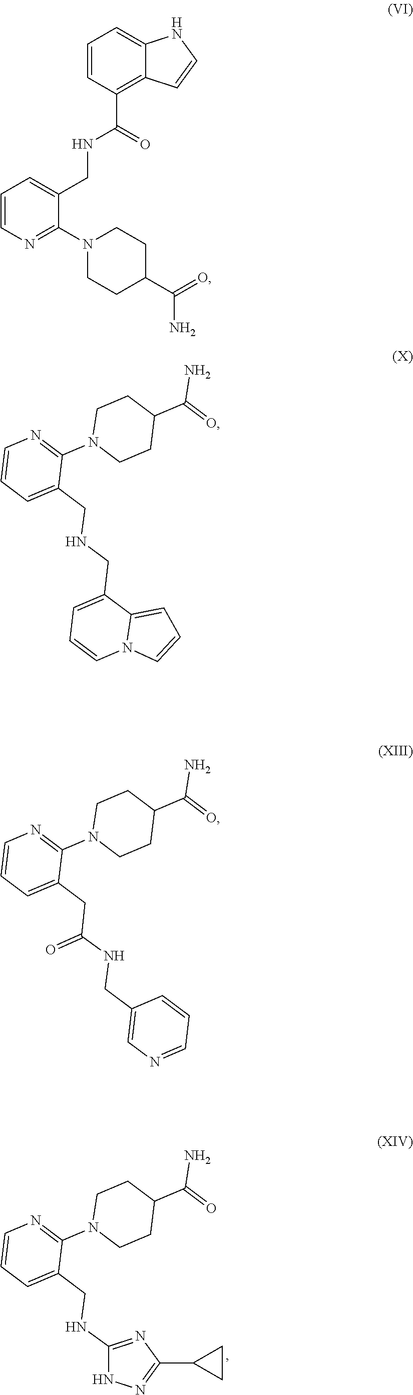

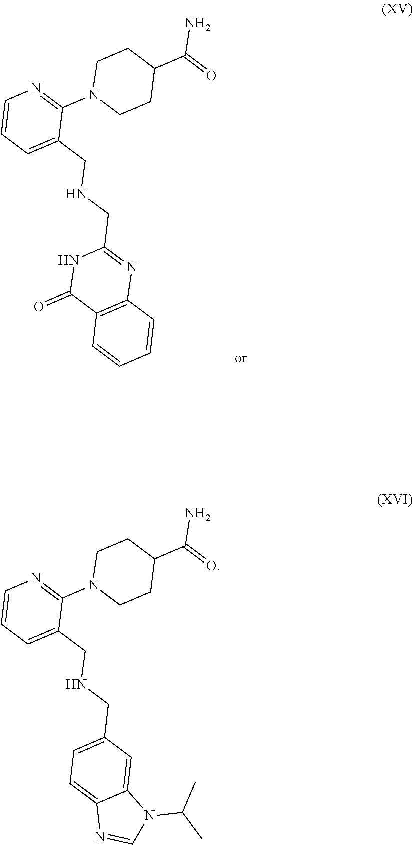 [SMILES: NC(=O)C1CCN(c2ncccc2CNCc2nc3ccccc3c(=O)[nH]2)CC1 ; CC(C)n1cnc2ccc(CNCc3cccnc3N3CCC(C(N)=O)CC3)cc21]

In some embodiments, the compound is a compound of Formula VI or XIII.

In some embodiments, the compound is a compound of any one of Formula IV-XVI; especially a compound of Formula V or XI.

In another aspect, there is provided a compound of Formula II or a pharmaceutically acceptable salt thereof:

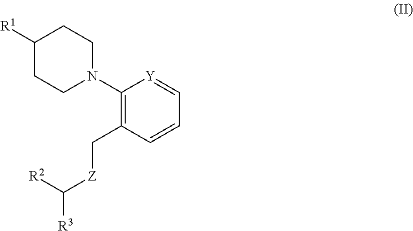

wherein:
R¹ is selected from the group consisting of H, hydroxy, optionally substituted C₁₋₆ alkyl, —NH₂, —C(O)—NH₂, optionally substituted —C(O)—C₁₋₆ alkyl, halogen, —C(OH)—NH₂, —C(O)—OH, —CH₂—NH₂, —C(O)H and optionally substituted C₁₋₆ alkoxy;
R² is selected from the group consisting of H, ═O, hydroxy, halogen, optionally substituted C₁₋₆ alkyl and optionally substituted C₁₋₆ alkoxy;
R³ is selected from the group consisting of optionally substituted C₆₋₁₈ aryl, optionally substituted 5- to 18-membered heteroaryl, optionally substituted C₅₋₁₈ cycloalkyl and optionally substituted 5- to 18-membered heterocycloalkyl;
X is selected from the group consisting of C and N;
Y is selected from the group consisting of C and N; and
Z is selected from the group consisting of C, O, N and S.

In some embodiments, R¹ is selected from the group consisting of —C(O)—NH₂, optionally substituted —C(O)—C₁₋₆ alkyl, —C(OH)—NH₂ and —C(O)—OH; especially —C(O)—NH₂.

In some embodiments, R² is H or ═O.

In particular embodiments, R³ is selected from the group consisting of optionally substituted C₆₋₁₈ aryl and optionally substituted 5- to 18-membered heteroaryl; especially optionally substituted especially 5- to 18-membered heteroaryl. In particular embodiments, R³ is optionally substituted 5- to 10-membered heteroaryl.

In particular embodiments, R³ is:

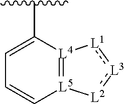

wherein L¹, L², L³, L⁴ and L⁵ are independently selected from the group consisting of C, O, N and S. In particular embodiments, one or two of L¹, L², L³, L⁴ and L⁵ are independently selected from the group consisting of O, N and S and the remainder are C. In specific embodiments, R³ is:

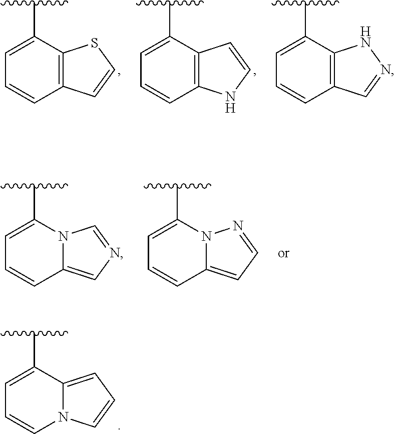

In specific embodiments, R³ is:

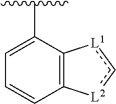

wherein:
L¹ and L² are independently selected from the group consisting of C, O, N and S. In particular embodiments, L¹ is C and L² is N or L¹ is S and L² is C.

In specific embodiments, R³ is:

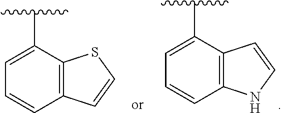 [SMILES: *c1cccc2ccsc12 ; *c1cccc2[nH]ccc12]

In some embodiments, X is N; Y is N; and/or Z is C or N, especially N.

In some embodiments, the compound is a compound of Formula IV or V:

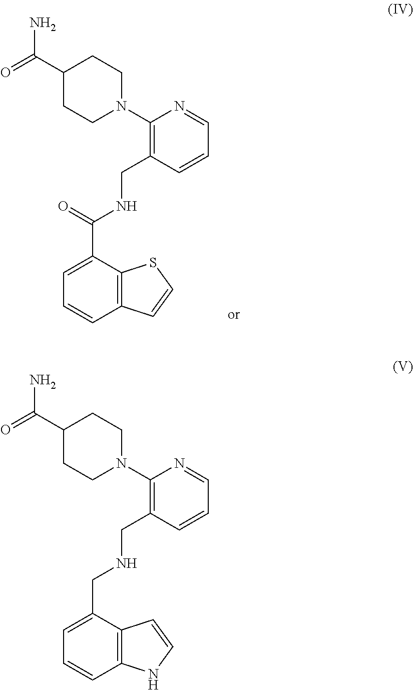 [SMILES: NC(=O)C1CCN(c2ncccc2CNC(=O)c2cccc3ccsc23)CC1 ; NC(=O)C1CCN(c2ncccc2CNCc2cccc3[nH]ccc23)CC1]

or a pharmaceutically acceptable salt thereof.

In another aspect, there is provided a composition comprising a compound or a pharmaceutically acceptable salt thereof of the invention and a pharmaceutically acceptable carrier or diluent.

In a further aspect, there is provided a compound or a pharmaceutically acceptable salt thereof of the invention for use in therapy.

In yet another aspect, there is provided a method of inhibiting NSP5 activity, comprising, consisting or consisting essentially of contacting NSP5 or a biologically active fragment or variant thereof with a compound or a pharmaceutically acceptable salt thereof of the invention.

In some embodiments, the NSP5 is a coronavirus NSP5. The NSP5 may comprise the amino acid sequence set forth in SEQ ID NO:1 or a sequence at least about 80% identical thereto. In a particular embodiment, the NSP5 is the SARS-CoV-2 NSP5.

Further provided is a method of treating or inhibiting the development of a coronavirus infection in a subject, comprising, consisting or consisting essentially of administering a compound or a pharmaceutically acceptable salt thereof of the invention to the subject.

In some embodiments, the coronavirus is capable of causing SARS.

In another aspect, there is provided a method for treating or inhibiting the development of neurological symptoms of, and/or neurological complications or impairments associated with, a coronavirus infection in a subject, comprising, consisting or consisting essentially of administering a compound or a pharmaceutically acceptable salt thereof of the invention to the subject.

The neurological symptoms, complications or impairments may include, for example, headache, fatigue, dizziness, difficulty sleeping, anxiety, depression, impaired or loss of smell (anosmia), impaired or loss of taste (dysgeusia), and cognitive challenges and/or deficits such as loss of memory, impaired concentration, poor attention, confusion difficulties with thinking and difficulties with decision making.

The neurological symptoms, complications or impairments may be experienced during the acute phase of a coronavirus infection. The neurological symptoms, complications or impairments may be experienced during recovery from a coronavirus infection. The neurological symptoms, complications or impairments may be experienced in a chronic phase following a coronavirus infection and may be irrespective of viral status.

In a particular embodiment, the coronavirus infection is a SARS-CoV-2 infection.

In another aspect, there is provided a method for treating or inhibiting the development or progression of long COVID or one or more symptoms thereof (also referred to as post COVID syndrome) in a subject following SARS-CoV-2 infection, comprising, consisting or consisting essentially of administering a compound or a pharmaceutically acceptable salt thereof of the invention to the subject.

The symptoms or manifestations of the long COVID may include, for example, fatigue, heart symptoms or conditions (such as myocarditis), digestive symptoms including diarrhea and stomach pain, joint or muscle pain, and one or more neurological symptoms, complications or impairments.

The neurological symptoms, complications or impairments may include, for example, headache, fatigue, dizziness, difficulty sleeping, anxiety, depression, impaired or loss of smell (anosmia), impaired or loss of taste (dysgeusia), and cognitive challenges and/or deficits such as loss of memory, impaired concentration, poor attention, confusion difficulties with thinking and difficulties with decision making.

In another aspect, there is provided a method of treating an acute inflammatory condition in a subject, wherein the condition is associated with a coronavirus infection, comprising, consisting or consisting essentially of administering a compound or a pharmaceutically acceptable salt thereof of the invention to the subject.

In some embodiments, the acute inflammatory condition is associated with presence of cytokine release syndrome (CRS) or a cytokine storm, a multisystem inflammatory syndrome in children (MIS-C), a systemic inflammatory response syndrome (SIRS), ARDS and/or SARS.

In a further aspect, there is provided a method of treating CRS or a cytokine storm in a subject, wherein the CRS or cytokine storm is associated with a coronavirus infection, comprising, consisting or consisting essentially of administering a compound or a pharmaceutically acceptable salt thereof of the invention to the subject.

In some embodiments, the CRS or cytokine storm comprises an elevation of at least 50% compared to basal state of one or more cytokines selected from IFN-γ, IFN-β, TNF-α, IL-1β, IL-6, IL-17A, CCL3 and CXCL2.

In particular embodiments, the subject has CRS and has one or more symptoms selected from fever, fatigue, loss of appetite, muscle and joint pain, nausea, vomiting, diarrhea, rashes, fast breathing, rapid heartbeat, low blood pressure, seizures, headache, confusion, delirium, hallucinations, tremor, loss of consciousness or loss of coordination. In some embodiments, the subject has a cytokine storm and has one or more symptoms selected from high fever, swelling and redness, extreme fatigue, nausea, bleeding, clotting, internal organ injury, and shock, or any combination thereof.

In some embodiments, the CRS or cytokine storm is associated with a MIS-C, a SIRS, ARDS or SARS.

In yet another aspect, there is provided a method of treating SARS in a subject, wherein the SARS is associated with a coronavirus infection, comprising, consisting or consisting essentially of administering a compound or a pharmaceutically acceptable salt thereof of the invention to the subject.

In some embodiments, the subject may have one or more symptoms selected from acute febrile illness, malaise, fatigue, headache, dizziness, flushing, fever, diarrhea, nausea, vomiting, coughing including dry coughing, sore throat, runny nose, nasal congestion, production of pro-inflammatory mediators, joint or muscle pain, impaired or loss of taste (dysgeusia), impaired or loss of smell (anosmia), vascular leakage and organ failure, or any combination thereof.

In particular embodiments, the SARS is associated with presence of CRS or a cytokine storm, a MISC-C, a SIRS.

In some embodiments of any one of the methods described above, the coronavirus is a betacoronavirus, in particular a lineage A betacoronavirus, a lineage B betacoronavirus, a lineage C betacoronavirus and a lineage D betacoronavirus.

In particular embodiments of any one of the methods described above, the betacoronavirus is a lineage B betacoronavirus, in particular SARS-CoV or SARS-CoV-2, more particularly SARS-CoV-2. In alternative embodiments of any one of the methods described above, the betacoronavirus is a lineage C betacoronavirus, such as MERS-CoV.

In some embodiments of any one of the methods described above, the compound is administered orally, intranasally, by inhalation, or by infusion (typically intravenous infusion).

Also provided is a compound or a pharmaceutically acceptable salt thereof of the invention for use in treating or inhibiting the development of a coronavirus infection in a subject.

In another aspect, there is provided a compound or a pharmaceutically acceptable salt thereof of the invention for use in treating or inhibiting the development of neurological symptoms of, and/or neurological complications or impairments associated with, a coronavirus infection.

In another aspect, there is provided a compound or a pharmaceutically acceptable salt thereof of the invention for use in treating or inhibiting the development or progression of long COVID, or one or more symptoms thereof, following SARS-CoV-2 infection.

In another aspect, there is provided a compound or a pharmaceutically acceptable salt thereof of the invention for use in treating an acute inflammatory condition in a subject, wherein the condition is associated with a coronavirus infection.

In a further aspect, there is provided a compound or a pharmaceutically acceptable salt thereof of the invention for use in treating CRS or a cytokine storm in a subject, wherein the CRS or cytokine storm is associated with a coronavirus infection.

In a still further aspect, there is provided a compound or a pharmaceutically acceptable salt thereof of the invention for use in treating SARS in a subject, wherein the SARS is associated with a coronavirus infection.

Also provided is a use of a compound or a pharmaceutically acceptable salt thereof of the invention in the manufacture of a medicament for inhibiting NSP5 activity.

In another aspect, there is provided a use of a compound or a pharmaceutically acceptable salt thereof of the invention in the manufacture of a medicament for treating or inhibiting the development of a coronavirus infection in a subject.

In another aspect, there is provided a use of a compound or a pharmaceutically acceptable salt thereof of the invention in the manufacture of a medicament for treating or inhibiting the development of neurological symptoms of, and/or neurological complications or impairments associated with, a coronavirus infection.

In another aspect, there is provided a use of a compound or a pharmaceutically acceptable salt thereof of the invention in the manufacture of a medicament for treating or inhibiting the development or progression of long COVID, or one or more symptoms thereof, following SARS-CoV-2 infection.

In a further aspect, there is provided a use of a compound or a pharmaceutically acceptable salt thereof of the invention in the manufacture of a medicament for treating an acute inflammatory condition in a subject, wherein the condition is associated with a coronavirus infection.

In yet another aspect, there is provided a use of a compound or a pharmaceutically acceptable salt thereof of the invention in the manufacture of a medicament for treating CRS or a cytokine storm in a subject, wherein the CRS or cytokine storm is associated with a coronavirus infection.

In a still further aspect, there is provided a use of a compound or a pharmaceutically acceptable salt thereof of the invention in the manufacture of a medicament for treating SARS in a subject, wherein the SARS is associated with a coronavirus infection.

### BRIEF DESCRIPTION OF THE DRAWINGS

Embodiments of the present invention are described herein, by way of non-limiting example only, with reference to the following figures.

FIG. 1 is an image showing the docking pose for compound 2 binding to NSP5.

FIG. 2 is an image showing the docking pose for compound 22 binding to NSP5.

FIG. 3 is a photographic image of fixed primary neurons following treatment with AAV9-GFP, AAV9-NSP5-V5 and AAV9-NSP5 C145A-V5. Fixed cells were stained with DAPI (blue), V5 (green) and βIII-tubulin (red).

FIG. 4 is a photographic image of fixed primary neurons following treatment with AAV9-NSP5-V5 and incubation with 0.4, 2, 10 or 50 μM of compound 2 (C2). Fixed cells were stained with DAPI (blue), V5 (green) and βIII-tubulin (red). Images represent three replicates.

FIG. 5 is a photographic image of fixed primary neurons following treatment with AAV9-NSP5-V5 and incubation with 0.4, 2, 10 or 50 μM of compound 22 (C22). Fixed cells were stained with DAPI (blue), V5 (green) and βIII-tubulin (red). Images represent three replicates.

FIG. 6 is a graphical representation of the effect of compounds 2 (C2) and 22 (C22) on neuronal cell death in the presence of NSP5.

FIG. 7 is a graphical representation of the effect of compounds 2 (C2) and 22 (C22) on neuronal cell death in the presence of NMDA.

FIG. 8 is a graphical representation of the effect of compounds 2 (C2) and 22 (C22) on neuronal cell death in the presence of H₂O₂.

FIG. 9 is a graphical representation of the effect of compounds 2 (C2) and 22 (C22) on neuronal cell death in the presence of staurosporine.

FIG. 10 is a graphical representation of the effect of 10 μM of compounds 22 and 32 to 40 (DA-CoV-022 and DA-CoV-032 to DA-CoV-040) on neuronal cell death in the presence of NSP5.

FIG. 11 shows the effect of compound 22 on the replication of Delta and Omicron variants of SARS-CoV-2. FIG. 11A is a flow chart showing the method for assessing the effect of compound 22 on viral replication, FIG. 11B is a graphical representation of the effect of compound 22 (50 μM) on the replication of the Delta variant of SARS-CoV-2 24, 48 and 72 hours after infection, and FIG. 11C is a graphical representation of the effect of compound 22 (50 μM) on the replication of the Omicron variant of SARS-CoV-2 24, 48 and 72 hours after infection.

FIG. 12 is a graphical representation of the total plasma concentrations of compound 22 in male C57BL/6 mice following intravenous (IV; 3 mg/kg) and intraperitoneal (IP; 10 mg/kg) administration.

FIG. 13 is a graphical representation of the unbound plasma concentrations of compound 22 in male C57BL/6 mice following IP administration at 10 mg/kg.

FIG. 14 is a series of graphs showing the effect of daily administration of compound 22 (DA-CoV-22; 10 mg/kg), Paxlovid (10 mg/kg nirmatrelvir: 3.33 mg/kg ritonavir) or a vehicle control (Veh) on the performance of 5 month old female C57BL/6 wild type mice injected with PHP.B hSyn-NSP5-V5 adeno-associated virus in the Rota-rod test 15-17 days post AAV injection (n=10/group). FIG. 14A presents the maximum time of five daily trials that the mice were able to stay on the accelerating rotating rod each day over a three-day period and FIG. 14B presents the average time of five daily trials that the mice were able to stay on the accelerating rotating rod each day over a three-day period.

FIG. 15 is a graphical representation of the effect of daily administration of compound 22 (DA-CoV-22; 10 mg/kg), Paxlovid (10 mg/kg nirmatrelvir: 3.33 mg/kg ritonavir) or a vehicle control (Veh) on the weight of 5 month old female C57BL/6 wild type mice injected with PHP.B hSyn-NSP5-V5 adeno-associated virus over a 21-day period (n=10/group).

FIG. 16 is a series of graphs showing the effect of daily administration of compound 22 (DA-CoV-22; 10 mg/kg), Paxlovid (10 mg/kg nirmatrelvir: 3.33 mg/kg ritonavir) or a vehicle control (Veh) on the performance of 5 month old female C57BL/6 wild type mice injected with PHP.B hSyn-NSP5-V5 adeno-associated virus in the hanging wire test (n=10/group). FIG. 16A presents the maximum time hanging upside down on the wire grid 14 days post AAV injection and FIG. 16B presents the maximum time hanging upside down on the wire grid 22 days post AAV injection.

FIG. 17 is a series of graphs showing the effect of daily administration of compound 22 (DA-CoV-22; 10 mg/kg), Paxlovid (10 mg/kg nirmatrelvir: 3.33 mg/kg ritonavir) or a vehicle control (Veh) on the performance of 5 month old female C57BL/6 wild type mice injected with PHP.B hSyn-NSP5-V5 adeno-associated virus in the elevated plus maze test 19 days post AAV injection (n=10/group). FIG. 17A presents the time in the protected closed arm of the apparatus, FIG. 17B presents the number of entries into the protected closed arm of the apparatus and FIG. 17C presents the total distance travelled in the apparatus.

### DETAILED DESCRIPTION OF THE INVENTION

### 1. Definitions

Unless defined otherwise, all technical and scientific terms used herein have the same meaning as commonly understood by those of ordinary skill in the art to which the invention belongs. Although any methods and materials similar or equivalent to those described herein can be used in the practice or testing of the present invention, preferred methods and materials are described. For the purposes of the present invention, the following terms are defined below.

The articles “a” and “an” are used herein to refer to one or to more than one (i.e. to at least one) of the grammatical object of the article. By way of example, “an element” means one element or more than one element.

By “about” is meant a quantity, level, value, number, frequency, percentage, dimension, size, amount, weight or length that varies by as much 15, 14, 13, 12, 11, 10, 9, 8, 7, 6, 5, 4, 3, 2 or 1% to a reference quantity, level, value, number, frequency, percentage, dimension, size, amount, weight or length.

The term “acute inflammatory condition” as used herein refers to a condition in which acute inflammation is present and represents a rapid, short-lived (minutes to days), relatively uniform response to acute injury characterised by accumulations of fluid, plasma proteins, and neutrophilic leukocytes. In acute inflammation, removal of the stimulus halts the recruitment of monocytes (which become macrophages under appropriate activation) into the inflamed tissue, and existing macrophages exit the tissue via lymphatics. Examples of injurious agents that cause acute inflammation include, but are not limited to, pathogens (e.g. bacteria, viruses, parasites), foreign bodies from exogenous (e.g. asbestos) or endogenous (e.g. urate crystals, immune complexes), sources, and physical (e.g. burns) or chemical (e.g. caustics) agents. Generally, the physiologic changes accompanying acute inflammation encompass four main features: (1) vasodilation, which results in a net increase in blood flow, is one of the earliest physical responses to acute tissue injury; (2) in response to inflammatory stimuli, endothelial cells lining the venules contract, widening the intracellular junctions to produce gaps, leading to increased vascular permeability, which permits leakage of plasma proteins and blood cells out of blood vessels; (3) inflammation often is characterised by a strong infiltration of leukocytes at the site of inflammation, particularly neutrophils (polymorphonuclear cells). These cells promote tissue damage by releasing toxic substances at the vascular wall or in uninjured tissue; and (4) fever, produced by pyrogens released from leukocytes in response to specific stimuli. Other conditions include a reduction in olfactory sensation and adverse effects on the cardiovascular system.

As used herein, the term “acute respiratory distress syndrome” or “ARDS” refers to a life-threatening lung condition that prevents enough oxygen from getting to the lungs and into the blood. ARDS is also referred to as noncardiogenic pulmonary oedema, increased-permeability pulmonary oedema, stiff lung, shock lung, or acute lung injury. ARDS can be caused by any major injury to the lung. Some common causes include, but are not limited to, breathing vomit into the lungs (aspiration), inhaling chemicals, lung transplant, pneumonia, septic shock (infection throughout the body) and trauma.

The terms “administration concurrently” or “administering concurrently” or “co-administering” and the like refer to the administration of a single composition containing two or more agents, or the administration of each agent as separate compositions and/or delivered by separate routes either contemporaneously or simultaneously or sequentially within a short enough period of time that the effective result is equivalent to that obtained when all such agents are administered as a single composition. By “simultaneously” is meant that the agents are administered at substantially the same time, and desirably together in the same composition. By “contemporaneously” it is meant that the agents are administered closely in time, e.g., one agent is administered within from about one minute to within about one day before or after another. Any contemporaneous time is useful. However, it will often be the case that when not administered simultaneously, the agents will be administered within about one minute to within about eight hours and suitably within less than about one to about four hours. When administered contemporaneously, the agents are suitably administered at the same site on the subject. The term “same site” includes the exact location, but can be within about 0.5 to about 15 centimeters, preferably from within about 0.5 to about 5 centimeters. The term “separately” as used herein means that the agents are administered at an interval, for example at an interval of about a day to several weeks or months. The agents may be administered in either order. The term “sequentially” as used herein means that the agents are administered in sequence, for example at an interval or intervals of minutes, hours, days or weeks. If appropriate the agents may be administered in a regular repeating cycle.

The term “agent” includes a compound that induces a desired pharmacological and/or physiological effect. The term also encompasses pharmaceutically acceptable and pharmacologically active ingredients of those compounds specifically mentioned herein including but not limited to salts, esters, amides, prodrugs, active metabolites, analogs and the like. When the above term is used, then it is to be understood that this includes the active agent per se as well as pharmaceutically acceptable, pharmacologically active salts, esters, amides, prodrugs, metabolites, analogs, etc. The term “agent” is not to be construed narrowly but extends to small molecules, proteinaceous molecules such as peptides, polypeptides and proteins as well as compositions comprising such molecules and genetic molecules such as RNA, DNA and mimetics and chemical analogs thereof as well as cellular agents.

The term “alkyl” as used herein as a group or part of a group refers to a straight or branched aliphatic hydrocarbon group, including a C₁₋₆ alkyl, C₁₋₅ alkyl, C₁₋₄ alkyl, C₁₋₃ alkyl and C₁₋₂ alkyl unless otherwise noted. Examples of alkyl include methyl, ethyl, n-propyl, iso-propyl, n-butyl, sec-butyl, iso-butyl, t-butyl, pentyl or hexyl; especially methyl or iso-propyl. The term “haloalkyl” as used herein refers to an alkyl group as defined above, wherein one or more of the hydrogen atoms are replaced by a halogen atom (e.g. F, Cl, Br or I). Examples of haloalkyl groups include —CF₃, —CH₂Cl and —CH₂CF₃; especially —CF₃. The term “alkylene” refers to divalent alkyl groups having from 1 to 6 carbon atoms, including from 1 to 5 carbon atoms, from 1 to 4 carbon atoms, from 1 to 3 carbon atoms and from 1 to 2 carbon atoms. Examples of such alkylene groups include methylene (—CH₂—), ethylene (—CH₂CH₂—), propylene, including n-propylene (—CH₂CH₂CH₂—), butylene, including n-butylene (—CH₂CH₂CH₂CH₂—), and the like.

As used herein, the term “alkoxy” refers to an alkyl-O— group in which alkyl is as defined herein. Unless herein before defined, “alkoxy” includes C₁₋₆ alkoxy, C₁₋₅ alkoxy, C₁₋₄ alkoxy, C₁₋₃ alkoxy or C₁₋₂ alkoxy, such as methoxy, ethoxy, n-propoxy, iso-propoxy, n-butoxy, sec-butoxy, iso-butoxy, t-butoxy, pentoxy or hexyloxy; especially methoxy.

As used herein, the term “and/or” refers to and encompasses any and all possible combinations of one or more of the associated listed items, as well as the lack of combinations when interpreted in the alternative (or).

The term “antiviral agent” as used herein refers to any agent with antiviral activity, i.e., the ability to inhibit or reduce the growth and/or kill a virus, e.g., by at least about 30%, at least about 40%, at least about 50%, at least about 75%, at least about 90% or more, as compared to in the absence of an antiviral agent. The term “antiviral agent” encompasses agents that are effective for inhibiting the formation and/or replication of a virus in a mammal. This includes agents that interfere with either host or viral mechanisms necessary for the formation and/or replication of a virus in a mammal. Such agents can be selected from immunomodulatory agents, inhibitors of a virus polymerase or inhibitors of another target in the virus life cycle.

The term “antagonist” and grammatical equivalents thereof as used herein refer to a molecule that partially or completely inhibits, by any mechanism, an effect of another molecule such as a receptor or intracellular mediator.

The term “aryl” as used herein refers to an unsaturated aromatic carbocyclic group having a single ring (e.g. phenyl) or multiple condensed rings (e.g. naphthyl or anthryl), including from 6-18 carbon atoms (and all integer carbon atoms therebetween). Where multiple rings are present, at least one of the rings is aromatic. In some embodiments, the aryl group is C₆₋₁₈ aryl, C₆₋₁₄ aryl or C₆₋₁₀ aryl. In preferred embodiments, the aryl group is phenyl, naphthyl or anthryl; especially phenyl or naphthyl. In particular embodiments, an aryl group may be optionally substituted by 1 to 3 substituents, for example, 1, 2 or 3 substituents, wherein the substituent is selected from C₁₋₆ alkyl, halogen, hydroxy, —NH₂, C₁₋₆alkoxy, —C(O)—OH or —C(O)—NH₂. In specific embodiments, an aryl group may be optionally substituted by 1 to 3 substituents, for example, 1, 2 or 3 substituents, wherein the substituent is selected from C₁₋₆ alkyl, halogen, hydroxy, —NH₂, C₁₋₆alkoxy, —C(O)—OH, —C(O)—NH₂, ═O, C₁₋₆ haloalkyl (e.g. CF₃) or C₃₋₆ cycloalkyl (e.g. cyclopropyl). In some embodiments, the aryl group is unsubstituted.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps. Thus, the use of the term “comprising” and the like indicates that the listed integers are required or mandatory, but that other integers are optional and may or may not be present. By “consisting of” is meant including, and limited to, whatever follows the phrase “consisting of”. Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified for the listed elements. Thus, the phrase “consisting essentially of” indicates that the listed elements are required or mandatory, but that other elements are optional and may or may not be present depending upon whether or not they affect the activity or action of the listed elements.

The term “cycloalkyl” refers to a saturated monocyclic or fused or spiro polycyclic carbocycle having from 5 to 18 carbon atoms. It includes monocyclic systems such as cyclopropyl and cyclohexyl, bicyclic systems such as declaim and polycyclic systems such as adamantine. In specific embodiments, the cycloalkyl is a C₅₋₁₈ cycloalkyl, C₅₋₁₄ cycloalkyl, C₅₋₁₀ cycloalkyl or C₅₋₉ cycloalkyl (and all integers therebetween). In particular embodiments, the cycloalkyl group is cyclopentyl, cyclohexyl, cycloheptyl, cyclooctyl, decalinyl or bicyclo[4.3.0]nonanyl. In particular embodiments, a cycloalkyl group may be optionally substituted by 1 to 3 substituents, for example, 1, 2 or 3 substituents, wherein the substituent is selected from C₁₋₆ alkyl, halogen, hydroxy, —NH₂, C₁₋₆alkoxy, —C(O)—OH or —C(O)—NH₂. In specific embodiments, a cycloalkyl group may be optionally substituted by 1 to 3 substituents, for example, 1, 2 or 3 substituents, wherein the substituent is selected from C₁₋₆ alkyl, halogen, hydroxy, —NH₂, C₁₋₆alkoxy, —C(O)—OH, —C(O)—NH₂, ═O, C₁₋₆ haloalkyl (e.g. CF₃) or C₃₋₆ cycloalkyl (e.g. cyclopropyl). In some embodiments, the cycloalkyl group is unsubstituted.

The term “cytokine release syndrome” or “CRS” refers to a form of SIRS that can be triggered by a variety of factors such as infections and certain drugs. It refers to cytokine storm syndromes (CSS) and occurs when large numbers of white blood cells are activated and release inflammatory cytokines, which in turn activate yet more white blood cells. CRS is also an adverse effect of some monoclonal antibody medications, as well as adoptive T-cell therapies. When occurring as a result of a medication, it is also known as an infusion reaction. The term cytokine storm is often used interchangeably with CRS but, despite the fact that they have similar clinical phenotype, their characteristics are different. When occurring as a result of a therapy, CRS symptoms may be delayed until days or weeks after treatment. Immediate-onset CRS is a cytokine storm, although severe cases of CRS have also been called cytokine storms.

As used herein, the term “cytokine storm” refers to an excessively activated cytokine cascade or hypercytokinemia, i.e., an excessive or uncontrolled release of pro-inflammatory cytokines, which can be associated with a wide variety of infectious and noninfectious diseases or disorders. Cytokine storm syndromes are associated with a group of disorders (such as, but not limited to, influenza, asthma, hantavirus pulmonary syndrome, SIRS, macrophage activation syndrome, SARS, COVID-19 and disseminated vascular coagulopathy), representing a variety of inflammatory causes. Typically, the primary symptoms of a cytokine storm are high fever, swelling and redness, extreme fatigue and nausea. In some cases, the immune reaction can result in bleeding, clotting, internal organ injury, or shock, and may be fatal.

By “derivative” is meant a molecule, such as a small molecule, that has been derived from the basic molecule by modification, for example by conjugation or complexing with other chemical moieties or by standard medicinal chemistry techniques as would be understood in the art. The term “derivative” also includes within its scope alterations that have been made to a parent molecule that provide for functionally equivalent molecules.

As used herein, the term “dosage unit form” refers to physically discrete units suited as unitary dosages for the subject to be treated, each unit containing a predetermined quantity of active material calculated to produce the desired therapeutic effect in association with the required pharmaceutically acceptable vehicle.

By “effective amount”, in the context of treating or preventing a condition is meant the administration of an amount of an agent or composition to an individual in need of such treatment or prophylaxis, either in a single dose or as part of a series, that is effective for the prevention of incurring a symptom, holding in check such symptoms, and/or treating existing symptoms, of that condition. The effective amount will vary depending upon the health and physical condition of the individual to be treated, the taxonomic group of individual to be treated, the formulation of the composition, the assessment of the medical situation, and other relevant factors. It is expected that the amount will fall in a relatively broad range that can be determined through routine trials. Non-limiting symptoms of viral infections (e.g. coronavirus infections) include acute febrile illness, malaise, fatigue, headache, flushing, diarrhea, nausea, vomiting, coughing including dry coughing, sore throat, runny nose, nasal congestion, and, in severe disease, symptoms of systemic inflammatory response syndrome including production of pro-inflammatory mediators, vascular leakage and organ failure.

The term “halo” or “halogen” as used herein refers to fluorine, chlorine, bromine or iodine, especially fluorine or chlorine, most especially fluorine.

The term “heteroaryl” is used herein to refer to an unsaturated aromatic cyclic group having a single ring or multiple condensed rings, having one or more heteroatoms as ring atoms, with the remainder of the ring atoms being carbon atoms. Suitable heteroatoms include nitrogen, oxygen and sulfur. Where multiple rings are present, at least one of the rings is aromatic. Heteroaryl encompasses 5- to 18-membered rings (and all integers therebetween), including 5- to 14-membered rings, 5- to 10-membered rings or 5- to 9-membered rings; especially 9-membered rings. Examples of “heteroaryl” as used herein include furyl, thienyl, pyrrolyl, imidazolyl, pyrazolyl, triazolyl, tetrazolyl, thiazolyl, oxazolyl, isoxazolyl, oxadiazolyl, thiadiazolyl, isothiazolyl, pyridyl, pyrimidinyl, quinolinyl, isoquinolinyl, quinolizinyl, benzofuryl, benzothienyl, indolyl, indolizinyl, isoindolyl, and indazolyl; especially pyrrolyl, furyl, thienyl, pyridyl, indolyl, indolizinyl, isoindolyl, quinolinyl, isoquinolinyl, quinolizinyl, benzofuryl or benzothienyl. In particular embodiments, a heteroaryl group may be optionally substituted by 1 to 3 substituents, for example, 1, 2 or 3 substituents, wherein the substituent is selected from C₁₋₆ alkyl, halogen, hydroxy, —NH₂, C₁₋₆alkoxy, —C(O)—OH or —C(O)—NH₂. In specific embodiments, a heteroaryl group may be optionally substituted by 1 to 3 substituents, for example, 1, 2 or 3 substituents, wherein the substituent is selected from C₁₋₆ alkyl, halogen, hydroxy, —NH₂, C₁₋₆alkoxy, —C(O)—OH, —C(O)—NH₂, ═O, C₁₋₆ haloalkyl (e.g. CF₃) or C₃₋₆ cycloalkyl (e.g. cyclopropyl). In some embodiments, the heteroaryl group is unsubstituted.

The term “heterocycloalkyl” refers to a saturated monocyclic, bicyclic or polycyclic group having one or more heteroatoms as ring atoms, with the remainder of the ring atoms being carbon atoms. Suitable heteroatoms include nitrogen, oxygen and sulfur. Heterocycloalkyl encompasses 5- to 18-membered rings (and all integers therebetween), including 5- to 14-membered rings, 5- to 10-membered rings or 5- to 9-membered rings; especially 9-membered rings. Examples of heterocycloalkyl as used herein include pyrrolidinyl, pyrrazolidinyl, imidazolidinyl, piperidinyl, thiazolidinyl, piperazinyl, morpholinyl, oxolanyl, oxanyl, thianyl, thiolanyl, azabicyclononanyl (e.g. azabicyclo[4.3.0]nonanyl), thiabicyclononanyl (e.g. thiabicyclo[4.3.0]nonanyl) or oxabicyclynonanyl (e.g. oxabicyclo[4.3.0]nonanyl). In particular embodiments, a heterocycloalkyl group may be optionally substituted by 1 to 3 substituents, for example, 1, 2 or 3 substituents, wherein the substituent is selected from C₁₋₆ alkyl, halogen, hydroxy, —NH₂, C₁₋₆alkoxy, —C(O)—OH or —C(O)—NH₂. In specific embodiments, a heterocycloalkyl group may be optionally substituted by 1 to 3 substituents, for example, 1, 2 or 3 substituents, wherein the substituent is selected from C₁₋₆ alkyl, halogen, hydroxy, —NH₂, C₁₋₆alkoxy, —C(O)—OH, —C(O)—NH₂, ═O, C₁₋₆ haloalkyl (e.g. CF₃) or C₃₋₆ cycloalkyl (e.g. cyclopropyl). In some embodiments, the heterocycloalkyl group is unsubstituted.

As used herein, the phrase “inhibit the development of” refers to a prophylactic treatment which increases the resistance of a subject to developing the disease or condition or, in other words, decreases the likelihood that the subject will develop the disease or condition as well as a treatment after the disease or condition has begun in order to reduce or eliminate it altogether or prevent it from becoming worse. This phrase also includes within its scope preventing the disease or condition from occurring in a subject which may be predisposed to the disease or condition but has not yet been diagnosed as having it.

The term “inhibitor” as used herein refers to an agent that decreases or inhibits at least one function or biological activity of a target molecule, such as NSP5. For example, an NSP5 inhibitor may decrease or reduce at least one function or biological activity of NSP5, such as the enzymatic activity, including the proteolytic activity. In this regard, an NSP5 inhibitor may inhibit cleavage of endogenous host cellular proteins and/or assembly of the viral replication transcription complex.

As used herein, the term “multisystem inflammatory syndrome in children” or “MIS-C” is a rare life-threatening illness where different body parts can become inflamed, including the heart, lungs, kidneys, brain, skin, eyes, or gastrointestinal organs. Children with MIS-C may have a fever and various symptoms, including abdominal (gut) pain, vomiting, diarrhea, neck pain, rash, bloodshot eyes or feeling extra tired.

The term “optionally substituted” as used throughout the specification denotes that the group may or may not be further substituted with one or more non-hydrogen substituent groups. In certain embodiments, the substituent groups are one or more groups independently selected from the group consisting of halogen, C₁₋₆ alkyl (e.g. methyl or ethyl), hydroxy, —NH₂, C₁₋₆alkoxy (e.g. methoxy or ethoxy), —C(O)—OH and —C(O)—NH₂. In some embodiments, the optional substituent groups are one or more groups independently selected from the group consisting of methyl, ethyl, halogen, hydroxy, —NH₂, methoxy, ethoxy, —C(O)—OH and —C(O)—NH₂; especially methyl, ethyl or halogen; most especially methyl. In certain embodiments, the substituent groups are one or more groups independently selected from the group consisting of halogen, C₁₋₆ alkyl (e.g. methyl, ethyl or propyl), hydroxy, —NH₂, C₁₋₆alkoxy (e.g. methoxy or ethoxy), —C(O)—OH, —C(O)—NH₂, ═O, C₁₋₆ haloalkyl (e.g. CF₃) and C₃₋₆ cycloalkyl (e.g. cyclopropyl). In some embodiments, the optional substituent groups are one or more groups independently selected from the group consisting of methyl, ethyl, propyl (e.g. iso-propyl), halogen, hydroxy, —NH₂, methoxy, ethoxy, —C(O)—OH, —C(O)—NH₂, ═O, —CF₃ and cyclopropyl; especially methyl, ethyl, iso-propyl, halogen (e.g. F), ═O, —CF₃ and cyclopropyl; most especially F, —CF₃, ═O, cyclopropyl, and iso-propyl. In exemplary embodiments, each optionally substituted group may be substituted with 1-3 substituents, including 1, 2 or 3 substituents, such as 1, 2 or 3 methyl groups.

The terms “patient”, “subject”, “host” or “individual” used interchangeably herein, refer to any subject, particularly a vertebrate subject, and even more particularly a mammalian subject, for whom therapy or prophylaxis is desired. Suitable vertebrate animals that fall within the scope of the present disclosure include, but are not restricted to, any member of the subphylum Chordata including primates (e.g. humans, monkeys and apes, and includes species of monkeys such as from the genus Macaca (e.g. cynomolgus monkeys such as Macaca fascicularis, and/or rhesus monkeys (Macaca mulatta)) and baboon (Papio ursinus), as well as marmosets (species from the genus Callithrix), squirrel monkeys (species from the genus Saimiri) and tamarins (species from the genus Saguinus), as well as species of apes such as chimpanzees (Pan troglodytes), rodents (e.g. mice rats, guinea pigs), lagomorphs (e.g. rabbits, hares), bovines (e.g. cattle), ovines (e.g. sheep), caprines (e.g. goats), porcines (e.g. pigs), equines (e.g. horses), canines (e.g. dogs), felines (e.g. cats), avians (e.g. chickens, turkeys, ducks, geese, companion birds such as canaries, budgerigars etc.), marine mammals (e.g. dolphins, whales), reptiles (e.g. snakes, frogs, lizards etc.), and fish. In specific embodiments, the subject is a primate such as a human, especially a human in need of treating or inhibiting the development of a coronavirus infection. However, it will be understood that the terms “patient,” “subject,” “host” or “individual” do not imply that symptoms are present.

By “pharmaceutically acceptable carrier” is meant a pharmaceutical vehicle comprised of a material that is not biologically or otherwise undesirable, i.e., the material may be administered to a subject along with the selected active agent without causing any or a substantial adverse reaction. Carriers may include excipients and other additives such as diluents, fillers, detergents, colouring agents, wetting or emulsifying agents, pH buffering agents, preservatives and the like as discussed in detail herein.

Similarly, a “pharmacologically acceptable” salt, solvate, ester or prodrug of a compound as provided herein is a salt, solvate, ester or prodrug that is not biologically or otherwise undesirable.

The terms “reduce”, “inhibit”, “decrease”, “prevent”, and grammatical equivalents when used in reference to the level of a substance and/or phenomenon in a first sample relative to a second sample, mean that the quantity of substance and/or phenomenon in the first sample is lower than in the second sample by any amount that is statistically significant using any art-accepted statistical method of analysis. When these terms are used to refer to the action of a compound or agent, the first sample may be a sample in the presence of the compound or agent and the second sample may be a comparative sample without the compound or agent. In one embodiment, the reduction may be determined subjectively, for example when a patient refers to their subjective perception of disease symptoms, such as pain, headache, fatigue, nausea, motor symptoms, coughing, sore throat, nasal congestion, runny nose, etc. In another embodiment, the reduction may be determined objectively, for example when the amount of virus (e.g. viral load) in a sample from a patient is lower than in an earlier sample from the patient. In another embodiment, the quantity of substance and/or phenomenon in the first sample is at least 10% lower than the quantity of the same substance and/or phenomenon in a second sample. In another embodiment, the quantity of the substance and/or phenomenon in the first sample is at least 25% lower than the quantity of the same substance and/or phenomenon in a second sample. In yet another embodiment, the quantity of the substance and/or phenomenon in the first sample is at least 50% lower than the quantity of the same substance and/or phenomenon in a second sample. In a further embodiment, the quantity of the substance and/or phenomenon in the first sample is at least 75% lower than the quantity of the same substance and/or phenomenon in a second sample. In yet another embodiment, the quantity of the substance and/or phenomenon in the first sample is at least 90% lower than the quantity of the same substance and/or phenomenon in a second sample.

As used herein, the terms “salts” and “prodrugs” include any pharmaceutically acceptable salt, ester, hydrate or any other compound which, upon administration to the recipient, is capable of providing (directly or indirectly) a compound of the invention, or an active metabolite or residue thereof. The term “pharmaceutically acceptable salts” refers without limitation to derivatives of the disclosed compounds wherein the parent compound is modified by converting an existing acid or base moiety to its salt form (e.g. by reacting the free base group with a suitable organic acid). Examples of pharmaceutically acceptable salts include, but are not limited to, mineral or organic acid salts of basic residues such as amines; alkali or organic salts of acidic residues such as carboxylic acids; and the like. Representative acid addition salts include acetate, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate and valerate salts, and the like. Representative alkali or alkaline earth metal salts include sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like. The pharmaceutically acceptable salts of the present invention include the conventional non-toxic salts of the parent compound formed, for example, from non-toxic inorganic or organic acids. The pharmaceutically acceptable salts can be synthesised from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. In particular embodiments, the salt is the sodium salt. Lists of suitable salts are found in, for example, Remington: The Science and Practice of Pharmacy, Adeboye Adejare and Joseph Remington (Ed), Academic Press, London, 23^{rd} Edition, 2021; Stahl and Wermuth (2002) Pharmaceutical Salts: Properties, Selection, and Use, Wiley-VCH; and Berge et al. (1977) Journal of Pharmaceutical Science, 66: 1-19, each of which is incorporated herein by reference in its entirety.

As used herein, the term “systemic inflammatory response syndrome” or “SIRS” refers to a clinical response arising from a non-specific insult with two or more of the following measureable clinical characteristics; a body temperature greater than 38 °C or less than 36 °C, a heart rate greater than 90 beats per minute, a respiratory rate greater than 20 per minute, a white blood cell count (total leukocytes) greater than 12,000 per mm³ or less than 4,000 per mm³, or a band neutrophil percentage greater than 10%. From an immunological perspective, it may be seen as representing a systemic response to an infectious (e.g. pathogenic microbe) or non-infectious insult (e.g. major surgery) or systemic inflammation. Confirmation of infection can be determined using any suitable procedure known in the art, illustrative examples of which include blood culture, nucleic acid detection (e.g. PCR), mass spectroscopy, immunological detection (e.g. ELISA), isolation of bacteria from infected cells, cell lysis and imaging techniques such as electron microscopy.

As used herein, the terms “treatment”, “treating”, and the like, refer to obtaining a desired pharmacologic and/or physiologic effect. The effect may be therapeutic in terms of a partial or complete cure for a disease or condition (e.g. a viral infection) and/or adverse effect attributable to the disease or condition. These terms also cover any treatment of a condition or disease in a subject, particularly in a human, and include: (a) inhibiting the disease or condition, i.e. arresting its development; or (b) relieving the disease or condition, i.e. causing regression of the disease or condition.

Each embodiment described herein is to be applied mutatis mutandis to each and every embodiment unless specifically stated otherwise.

### 2. Abbreviations

The following abbreviations are used throughout the application:
NSP5=non-structural protein 5
HCl=hydrochloric acid
NCS=N-chlorosuccinimide
THF=tetrahydrofuran
Pd(PPh₃)₄=tetrakis(triphenylphosphine)palladium(0)
DIV=days in vitro
GFP=green fluorescent protein
AAV=adeno-associated virus
DAPI=4′-6-diamidino-2-phenylindole
CRS=cytokine release syndrome
SIRS=systemic inflammatory response syndrome
SARS=severe acute respiratory syndrome
ARDS=acute respiratory distress syndrome
MIS-C=multisystem inflammatory syndrome in children
DMSO=dimethyl sulfoxide
AR=analytical replicate
HATU=1-[bis(dimethylamino)methylene]-1H-1,2,3-triazolo[4,5-b]pyridinium 3-oxid hexafluorophosphate
PyBOP=benzotriazole-1-yl-oxy-tris-pyrrolidino-phosphonium hexafluorophosphate
DIPEA=N,N-diisopropylethylamine
OAc=acetyloxy
CH₃CN=acetonitrile
DMF=dimethylformamide
RuPhos Pd G4=[dicyclohexyl(2′,6′-diisopropoxy-2-biphenylyl)phosphine-κP](methanesulfonatato-κO)[2′-(methylamino-κN)-2-biphenylyl-KC²]palladium
AcOH=acetic acid
MeOH=methanol
NaBH₃CN=sodium cyanoborohydride

### 3. NPS5

This invention is based on the discovery of compounds that inhibit viral non-structural protein 5 (NSP5). NSP5 is also known as main protease, Mpro, 3C-like protease or 3CLpro. NSP5 is a cysteine protease responsible for cleavage of viral polyproteins to release individual non-structural proteins. NSP5 has been shown to play a role in viral entry into host cells and, more recently, has been found to cleave host proteins, impacting on host cell function.

The NSP5 against which compounds of the invention are directed may be encoded by, or derived from a coronavirus. For example, the coronavirus may be a betacoronavirus, in particular a lineage A betacoronavirus, a lineage B betacoronavirus, a lineage C betacoronavirus and a lineage D betacoronavirus. Representative lineage B betacoronaviruses include SARS-CoV and SARS-CoV-2. A representative lineage C betacoronavirus is MERS-CoV.

The NSP5 may comprise or consist of the amino acid sequence set forth in SEQ ID NO:1 or a sequence at least about 80% identical thereto. The amino acid sequence of SEQ ID NO:1 (the mature sequence of the SARS-CoV-2 NSP5) is set forth below:

SGFRKMAFPSGKVEGCMVQVTCGTTTLNGLWLDDVVYCPRHVICTSEDML
NPNYEDLLIRKSNHNFLVQAGNVQLRVIGHSMQNCVLKLKVDTANPKTPK
YKFVRIQPGQTFSVLACYNGSPSGVYQCAMRPNFTIKGSFLNGSCGSVGF
NIDYDCVSFCYMHHMELPTGVHAGTDLEGNFYGPFVDRQTAQAAGTDTTI
TVNVLAWLYAAVINGDRWFLNRFTTTLNDFNLVAMKYNYEPLTQDHVDIL
GPLSAQTGIAVLDMCASLKELLQNGMNGRTILGSALLEDEFTPFDVVRQC
SGVTFQ

The NSP5 may comprise or consist of an amino acid sequence at least about 80% identical to the sequence of SEQ ID NO:1. For example, the NSP5 may comprise or consist of a sequence that shares at least about 80%, 81%, 82%, 83%, 84%, 85%, 86%, 87%, 88%, 89%, 90%, 91%, 92%, 93%, 94%, 95%, 96%, 97%, 98% or 99% sequence identity with the NSP5 of SEQ ID NO: 1.

Thus, homologues and variants of the SARS-CoV-2 NSP5 are contemplated herein. A homologue is typically a protein or polypeptide from a different virus or organism sharing qualitative biological function or activity in common with the corresponding SARS-CoV-2 NSP5 disclosed herein. The term “variant” as used herein refers to substantially similar sequences. Generally, variant polypeptides or proteins possess qualitative biological function or activity in common with the polypeptide or protein from which they are derived. By way of example, variants and homologues may comprise the addition, deletion, substitution of one or more amino acids relative to the SARS-CoV-2 NSP5 disclosed herein.

### 4. Compounds

This invention is based on the discovery of compounds that inhibit NSP5, in particular SARS-CoV-2 NSP5. The inventors have conceived that such compounds will be useful for: treating and inhibiting the development of conditions associated with NSP5 activity, including a coronavirus infection; treating or inhibiting the development of conditions associated with coronavirus infections; treating or inhibiting the development of neurological symptoms of, and/or neurological complications or impairments associated with, a coronavirus infection; and treating or inhibiting the development or progression of long COVID following SARS-CoV-2 infection.

In one aspect, there is provided a compound of Formula I:

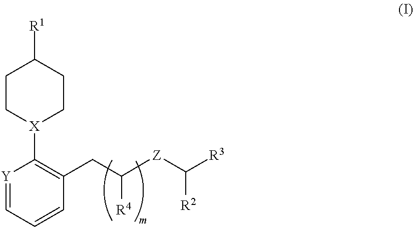

or a pharmaceutically acceptable salt thereof,
wherein:
R¹ is selected from the group consisting of H, hydroxy, optionally substituted C₁₋₆ alkyl, —NH₂, —C(O)—NH₂, optionally substituted —C(O)—C₁₋₆ alkyl, halogen, —C(OH)—NH₂, —C(O)—OH, —CH₂—NH₂, —C(O)H and optionally substituted C₁₋₆ alkoxy;
R² is selected from the group consisting of H, ═O, hydroxy, halogen, optionally substituted C₁₋₆ alkyl and optionally substituted C₁₋₆ alkoxy; and
R³ is selected from the group consisting of optionally substituted C₆₋₁₈ aryl, optionally substituted 5- to 18-membered heteroaryl, optionally substituted C₅₋₁₈ cycloalkyl, optionally substituted 5- to 18-membered heterocycloalkyl, or —C₁₋₆ alkylene-R⁵; or
R² and R³ together with the carbon atom to which they are attached form an optionally substituted C₆₋₁₈ aryl, optionally substituted 5- to 18-membered heteroaryl, optionally substituted C₅₋₁₈ cycloalkyl and optionally substituted 5- to 18-membered heterocycloalkyl;
R⁴ is selected from the group consisting of H, ═O, hydroxy, halogen, optionally substituted C₁₋₆ alkyl and optionally substituted C₁₋₆ alkoxy;
R⁵ is selected from the group consisting of optionally substituted C₆₋₁₈ aryl, optionally substituted 5- to 18-membered heteroaryl, optionally substituted C₅₋₁₈ cycloalkyl and optionally substituted 5- to 18-membered heterocycloalkyl;
m is 0 or 1;
X is selected from the group consisting of C and N;
Y is selected from the group consisting of C and N; and
Z is selected from the group consisting of C, O, N and S.

In some embodiments, the compound is other than a compound of Formula IV, V, VII, VIII, IX, XI and/or XII:

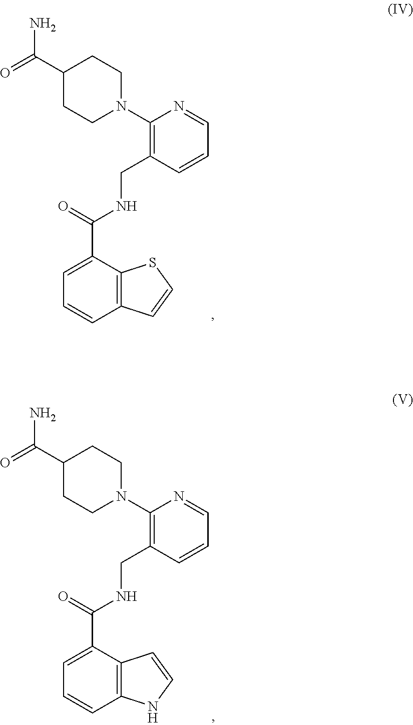 [SMILES: NC(=O)C1CCN(c2ncccc2CNC(=O)c2cccc3ccsc23)CC1 ; NC(=O)C1CCN(c2ncccc2CNC(=O)c2cccc3[nH]ccc23)CC1]

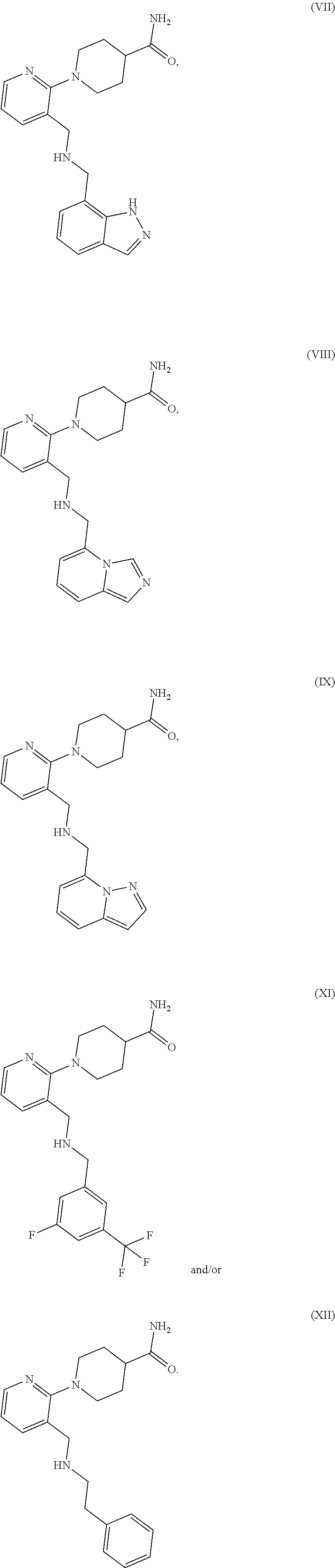 [SMILES: NC(=O)C1CCN(c2ncccc2CNCc2cccc3cn[nH]c23)CC1 ; NC(=O)C1CCN(c2ncccc2CNCc2cccc3cncn23)CC1 ; NC(=O)C1CCN(c2ncccc2CNCc2cccc3ccnn23)CC1 ; NC(=O)C1CCN(c2ncccc2CNCc2cc(F)cc(C(F)(F)F)c2)CC1 ; NC(=O)C1CCN(c2ncccc2CNCCc2ccccc2)CC1]

In some embodiments, R¹ is selected from the group consisting of H, hydroxy, optionally substituted C₁₋₆ alkyl (e.g. C₁₋₃ alkyl, especially methyl or ethyl), —NH₂, —C(O)—NH₂, optionally substituted —C(O)—C₁₋₆ alkyl (e.g. —C(O)—C₁₋₃ alkyl, especially acetyl or propionyl), halogen, —C(OH)—NH₂, C(O)—OH, —CH₂—NH₂, —C(O)H and optionally substituted C₁₋₆ alkoxy (e.g. C₁₋₃ alkoxy, especially methoxy or ethoxy); especially optionally substituted C₁₋₆ alkyl, —C(O)—NH₂, optionally substituted —C(O)—C₁₋₆ alkyl, —C(OH)—NH₂, —C(O)—OH, —CH₂—NH₂, —C(O)H and optionally substituted C₁₋₆ alkoxy. In particular embodiments, R¹ is selected from the group consisting of —C(O)—NH₂, optionally substituted —C(O)—C₁₋₆ alkyl, —C(OH)—NH₂ and —C(O)—OH; especially —C(O)—NH₂ and optionally substituted —C(O)—C₁₋₆ alkyl; most especially —C(O)—NH₂.

In some embodiments, R¹ is selected from the group consisting of H, hydroxy, C₁₋₆ alkyl, —NH₂, —C(O)—NH₂, —C(O)—C₁₋₆ alkyl, halogen, —C(OH)—NH₂, —C(O)—OH, —CH₂—NH₂, —C(O)H and C₁₋₆ alkoxy; especially C₁₋₆alkyl, —C(O)—NH₂, —C(O)—C₁₋₆ alkyl, —C(OH)—NH₂, —C(O)—OH, —CH₂—NH₂, —C(O)H and C₁₋₆ alkoxy. In particular embodiments, R¹ is selected from the group consisting of —C(O)—NH₂, —C(O)—C₁₋₆ alkyl, —C(OH)—NH₂ and —C(O)—OH; especially —C(O)—NH₂ and —C(O)—C₁₋₆ alkyl; most especially —C(O)—NH₂.

In particular embodiments, R¹ is —C(O)—NH₂.

In some embodiments, R² is selected from the group consisting of H, ═O, hydroxy, halogen, optionally substituted C₁₋₆ alkyl (e.g. C₁₋₃ alkyl, especially methyl or ethyl) and optionally substituted C₁₋₆ alkoxy (e.g. C₁₋₃ alkoxy, especially methoxy or ethoxy); especially H, ═O, hydroxy and halogen. In particular embodiments, R² is H or ═O.

In some embodiments, R² is selected from the group consisting of H, ═O, hydroxy, halogen, C₁₋₆ alkyl and C₁₋₆ alkoxy; especially H, ═O, hydroxy and halogen. In particular embodiments, R² is H or ═O.

In some embodiments, R³ is selected from the group consisting of optionally substituted C₆₋₁₈ aryl (e.g. phenyl, naphthyl or anthryl), optionally substituted 5- to 18-membered heteroaryl (e.g. pyrrolyl, furyl, thienyl, pyridyl, indolyl, indolizinyl, isoindolyl, quinolinyl, isoquinolinyl, quinolizinyl, benzofuryl, benzothienyl, indazolyl, pyridinyl, triazolyl, quinazolinyl or benzodiazolyl), optionally substituted C₅₋₁₈ cycloalkyl (e.g. cyclopentyl, cyclohexyl, decalinyl or bicyclo[4.3.0]nonanyl), optionally substituted 5- to 18-membered heterocycloalkyl (e.g. pyrrolidinyl, piperidinyl, oxolanyl, oxanyl, thianyl, thiolanyl, azabicyclononanyl, thiabicyclononanyl or oxabicyclynonanyl) and —C₁₋₆ alkylene-R⁵; especially optionally substituted C₆₋₁₈ aryl, optionally substituted 5- to 18-membered heteroaryl and —C₁₋₆ alkylene-R⁵. In particular embodiments, R³ is optionally substituted 5- to 18-membered heteroaryl or optionally substituted C₆₋₁₈ aryl. In particular embodiments, R³ is optionally substituted 5- to 10-membered heteroaryl or optionally substituted C₆₋₁₀ aryl; more especially optionally substituted benzothienyl, indolyl, indazolyl, pyridinyl, phenyl, triazolyl, quinazolinyl or benzodiazolyl.

In some embodiments, R³ is selected from the group consisting of C₆₋₁₈ aryl, 5- to 18-membered heteroaryl, C₅₋₁₈ cycloalkyl, 5- to 18-membered heterocycloalkyl and —C₁₋₆ alkylene-R⁵; especially C₆₋₁₈ aryl, 5- to 18-membered heteroaryl and —C₁₋₆ alkylene-R⁵. In particular embodiments, R³ is a 5- to 18-membered heteroaryl or a C₆₋₁₈ aryl; especially R³ is a 5- to 10-membered heteroaryl or a C₆₋₁₀ aryl; more especially benzothienyl, indolyl, indazolyl, pyridinyl, phenyl, triazolyl, quinazolinyl or benzodiazolyl.

In some embodiments, R³ is optionally substituted 5- to 10-membered heteroaryl. In particular embodiments, R³ is:

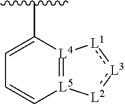

wherein L¹, L², L³, L⁴ and L⁵ are independently selected from the group consisting of C, O, N, NH, and S. In particular embodiments, one or two of L¹, L², L³, L⁴ and L⁵ are independently selected from the group consisting of O, N, NH and S and the remainder are C. In specific embodiments, R³ is:

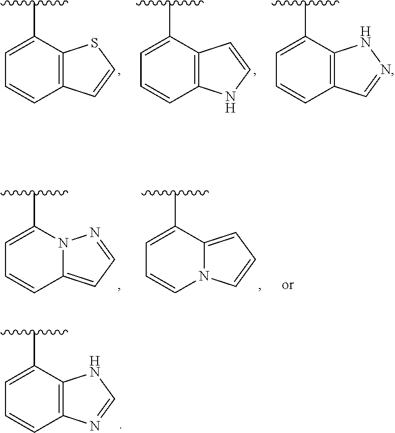

In particular embodiments, R³ is:

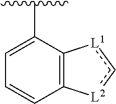

wherein:
L¹ and L² are independently selected from the group consisting of C, O, N and S.

For the avoidance of doubt, “ [symbol] ” refers to a single or a double bond.

In exemplary embodiments, L¹ is C and L² is selected from the group consisting of O, N and S; especially N or S. In alternative embodiments, L² is C and L¹ is selected from the group consisting of O, N and S; especially N or S. In particular embodiments, L¹ is C and L² is N, or L¹ is S and L² is C. In specific embodiments, R³ is:

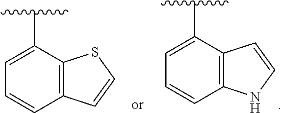 [SMILES: *c1cccc2ccsc12 ; *c1cccc2[nH]ccc12]

In some embodiments, R³ is —C₁₋₆ alkylene-R⁵, wherein R⁵ is selected from the group consisting of optionally substituted C₆₋₁₈ aryl, optionally substituted 5- to 18-membered heteroaryl, optionally substituted C₅₋₁₈ cycloalkyl and optionally substituted 5- to 18-membered heterocycloalkyl. In some embodiments, R⁵ is optionally substituted C₆₋₁₈ aryl and optionally substituted 5- to 18-membered heteroaryl. In particular embodiments, R⁵ is optionally substituted benzothienyl, indolyl, indazolyl, pyridinyl, phenyl, triazolyl, quinazolinyl or benzodiazolyl. In particular embodiments, R³ is C₁₋₃ alkylene-R⁵, especially methylene-R⁵. In particular embodiments, R³ is benzyl.

In some embodiments, R³ is selected from the group consisting of:

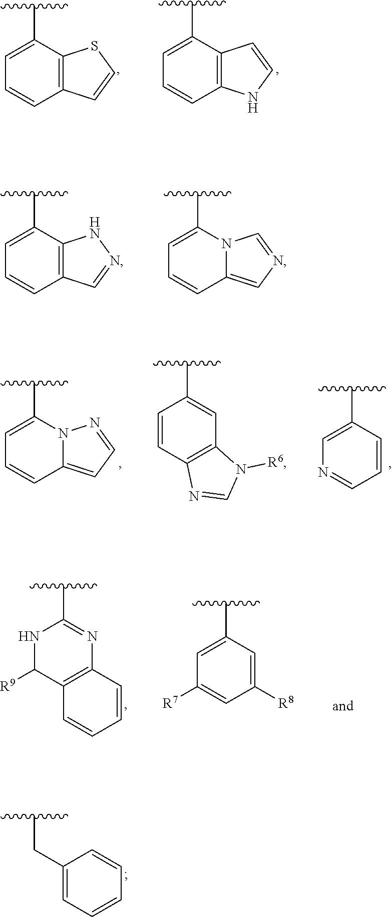

wherein R⁶ is selected from the group consisting of H, C₁₋₆ alkyl and halogen;
R⁷ is selected from the group consisting of H, C₁₋₆ alkyl, halogen and C₁₋₆ haloalkyl;
R⁸ is selected from the group consisting of H, C₁₋₆ alkyl, halogen and C₁₋₆ haloalkyl; and
R⁹ is selected from the group consisting of H, ═O, hydroxy, halogen and C₁₋₆ alkyl.

In some embodiments, R³ is selected from the group consisting of:

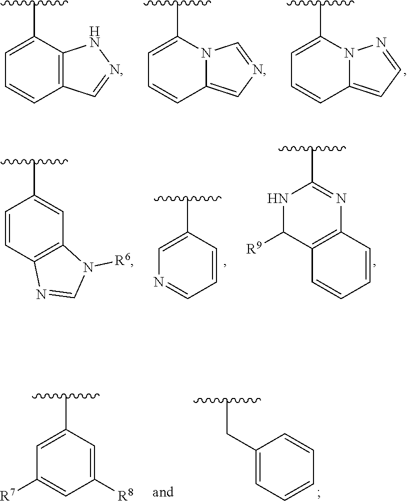

wherein R⁶, R⁷, R⁸ and R⁹ are as defined above.

In some embodiments, R⁶ is C₁₋₆ alkyl; especially —CH(CH₂)₂ (iso-propyl).

In some embodiments, R⁷ is C₁₋₆ haloalkyl; especially —CF₃.

In particular embodiments, R⁸ is halogen; especially F.

In some embodiments, R⁹ is ═O.

In alternative embodiments, R² and R³ together with the carbon atom to which they are attached form an optionally substituted 5- to 18-membered heteroaryl, such as optionally substituted benzothienyl, indolyl, indazolyl, pyridinyl, triazolyl, quinazolinyl or benzodiazolyl; especially optionally substituted triazolyl. In particular embodiments, R² and R³ together with the carbon atom to which they are attached form:

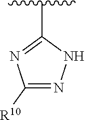

wherein R¹⁰ is selected from the group consisting of H, C₁₋₆ alkyl, C₃₋₆ cycloalkyl and halogen, and wherein

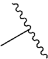

indicates the point of attachment to Z.

In particular embodiments, R¹⁰ is C₁₋₆ alkyl, C₃₋₆ cycloalkyl or halogen; especially C₃₋₆ cycloalkyl; more especially cyclopropyl.

In particular embodiments, m is 0.

In alternative embodiments, m is 1. In such embodiments, R⁴ is selected from the group consisting of H, ═O, hydroxy, halogen, optionally substituted C₁₋₆ alkyl (e.g. C₁₋₃ alkyl, especially methyl or ethyl) and optionally substituted C₁₋₆ alkoxy (e.g. C₁₋₃ alkoxy, especially methoxy or ethoxy); especially H, ═O, hydroxy and halogen. In particular embodiments, R⁴ is H or ═O; especially ═O.

In some embodiments, X is N. In alternative embodiments, X is C.

In some embodiments, Y is N. In alternative embodiments, Y is C.

In some embodiments, Z is C or N; especially N.

In particular embodiments, the compound is a compound of Formula IA:

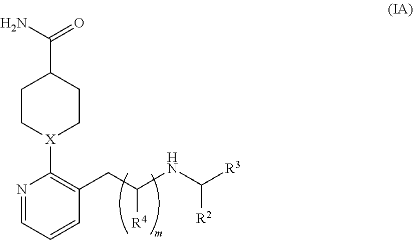

wherein R², R³, R⁴ and m are as described above for Formula I.

Suitable embodiments of R², R³, R⁴ and m are as discussed supra.

In particular embodiments, the compound is a compound of Formula IB:

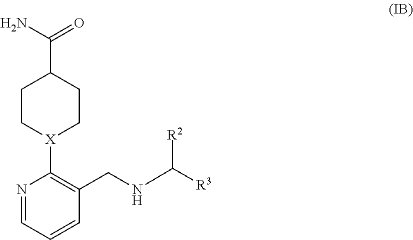

wherein R² and R³ are as described above for Formula I.

Suitable embodiments of R² and R³ are as discussed supra.

In particular embodiments, the compound is a compound of any one of Formula IV-XVI:

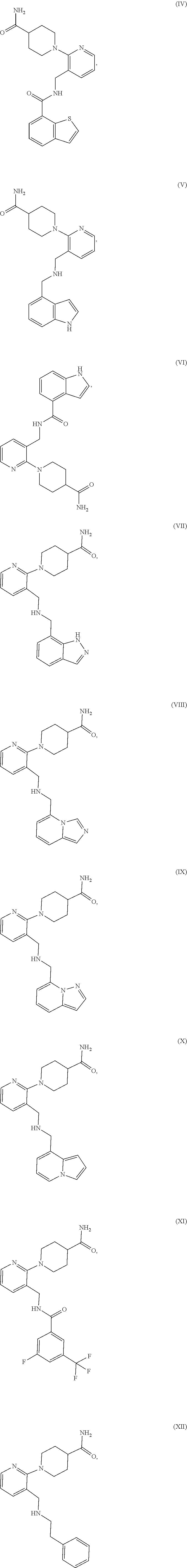

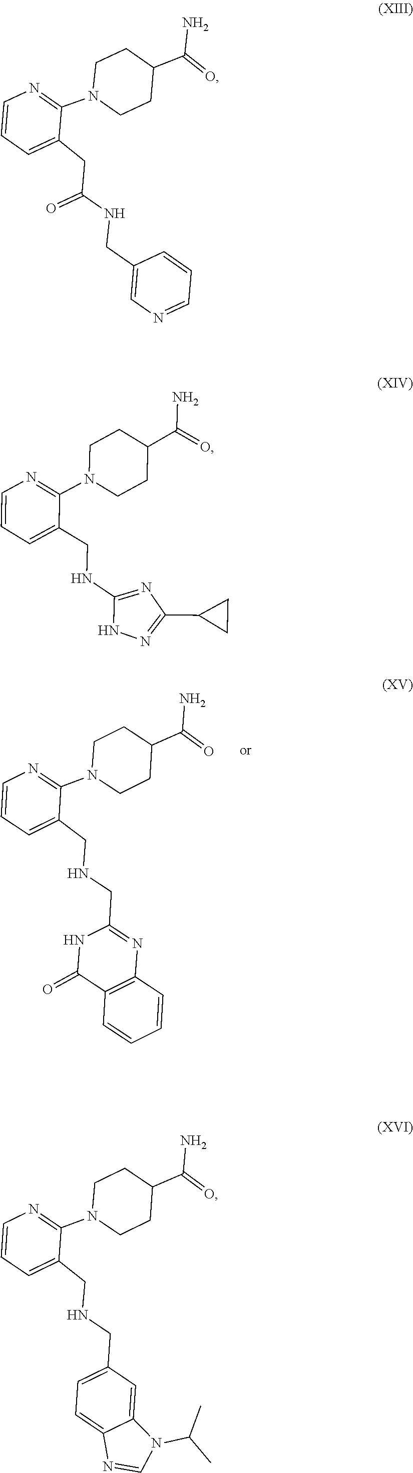 [SMILES: NC(=O)C1CCN(c2ncccc2CC(=O)NCc2cccnc2)CC1 ; NC(=O)C1CCN(c2ncccc2CNc2nc(C3CC3)n[nH]2)CC1 ; NC(=O)C1CCN(c2ncccc2CNCc2nc3ccccc3c(=O)[nH]2)CC1 ; CC(C)n1cnc2ccc(CNCc3cccnc3N3CCC(C(N)=O)CC3)cc21]

or a pharmaceutically acceptable salt thereof.

In some embodiments, the compound is a compound of any one of Formula VI-XVI. In some embodiments, the compound is a compound of Formula V or XI. In some embodiments, the compound is a compound of Formula VI or XIII.

In some embodiments, the compound is other than a compound of Formula IV and/or Formula V. In some embodiments, the compound is other than a compound of Formula IV, V, VII, VIII, IX, XI and/or XII. In some embodiments, the compound is other than a compound of Formula IV, V, VII, VIII, IX, XI, XII, XIV, XV and/or XVI.

In some embodiments, the compound is a compound of Formula II or a pharmaceutically acceptable salt thereof:

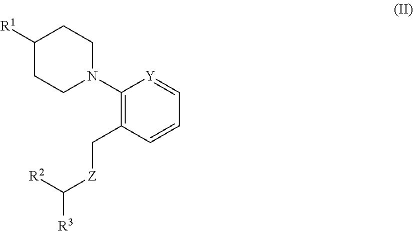

wherein:
R¹ is selected from the group consisting of H, hydroxy, optionally substituted C₁₋₆ alkyl, —NH₂, —C(O)—NH₂, optionally substituted —C(O)—C₁₋₆ alkyl, halogen, —C(OH)—NH₂, —C(O)—OH, —CH₂—NH₂, —C(O)H (i.e. formyl) and optionally substituted C₁₋₆ alkoxy;
R² is selected from the group consisting of H, ═O, hydroxy, halogen, optionally substituted C₁₋₆ alkyl and optionally substituted C₁₋₆ alkoxy;
R³ is selected from the group consisting of optionally substituted C₆₋₁₈ aryl, optionally substituted 5- to 18-membered heteroaryl, optionally substituted C₅₋₁₈ cycloalkyl and optionally substituted 5- to 18-membered heterocycloalkyl;
X is selected from the group consisting of C and N;
Y is selected from the group consisting of C and N; and
Z is selected from the group consisting of C, O, N and S.

Suitable embodiments of R¹ are as discussed supra for a compound of Formula I.

In some embodiments, R² is selected from the group consisting of H, ═O, hydroxy, halogen, optionally substituted C₁₋₆ alkyl (e.g. C₁₋₃ alkyl, especially methyl or ethyl) and optionally substituted C₁₋₆ alkoxy (e.g. C₁₋₃ alkoxy, especially methoxy or ethoxy); especially H, ═O, hydroxy and halogen. In particular embodiments, R² is H or ═O.

In some embodiments, R² is selected from the group consisting of H, ═O, hydroxy, halogen, C₁₋₆ alkyl and C₁₋₆ alkoxy; especially H, ═O, hydroxy and halogen. In particular embodiments, R² is H or ═O.

In some embodiments, R³ is selected from the group consisting of optionally substituted C₆₋₁₈ aryl (e.g. phenyl, naphthyl or anthryl), optionally substituted 5- to 18-membered heteroaryl (e.g. pyrrolyl, furyl, thienyl, pyridyl, indolyl, indolizinyl, isoindolyl, quinolinyl, isoquinolinyl, quinolizinyl, benzofuryl or benzothienyl), optionally substituted C₅₋₁₈ cycloalkyl (e.g. cyclopentyl, cyclohexyl, decalinyl or bicyclo[4.3.0]nonanyl) and optionally substituted 5- to 18-membered heterocycloalkyl (e.g. pyrrolidinyl, piperidinyl, oxolanyl, oxanyl, thianyl, thiolanyl, azabicyclononanyl, thiabicyclononanyl or oxabicyclynonanyl); especially optionally substituted C₆₋₁₈ aryl and optionally substituted 5- to 18-membered heteroaryl. In particular embodiments, R³ is optionally substituted 5- to 18-membered heteroaryl; more especially benzothienyl or indolyl. In particular embodiments, R³ is optionally substituted 5- to 18-membered heteroaryl or optionally substituted C₆₋₁₈ aryl; especially optionally substituted benzothienyl, indolyl, indazolyl, pyridinyl, phenyl, triazolyl, quinazolinyl or benzodiazolyl.

In some embodiments, R³ is selected from the group consisting of C₆₋₁₈ aryl, 5- to 18-membered heteroaryl, C₅₋₁₈ cycloalkyl and 5- to 18-membered heterocycloalkyl; especially C₆₋₁₈ aryl and 5- to 18-membered heteroaryl. In particular embodiments, R³ is 5- to 18-membered heteroaryl; more especially benzothienyl or indolyl. In some embodiments, R³ is benzothienyl, indolyl, indazolyl, pyridinyl, phenyl, triazolyl, quinazolinyl or benzodiazolyl.

In some embodiments, R³ is optionally substituted 5- to 10-membered heteroaryl or optionally substituted C₆₋₁₀ aryl.

In some embodiments, R³ is optionally substituted 5- to 10-membered heteroaryl. In particular embodiments, R³ is:

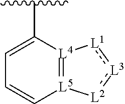

wherein L¹, L², L³, L⁴ and L⁵ are independently selected from the group consisting of C, O, N, NH, and S. In particular embodiments, one or two of L¹, L², L³, L⁴ and L⁵ are independently selected from the group consisting of O, N, NH and S and the remainder are C. In specific embodiments, R³ is:

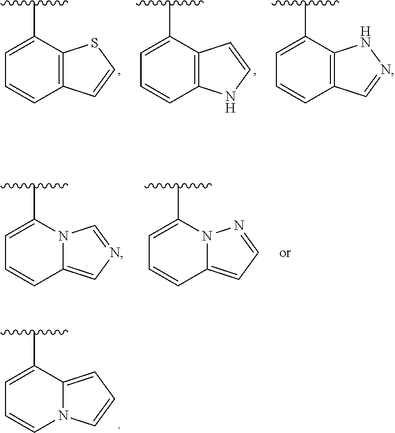

In some embodiments, R³ is:

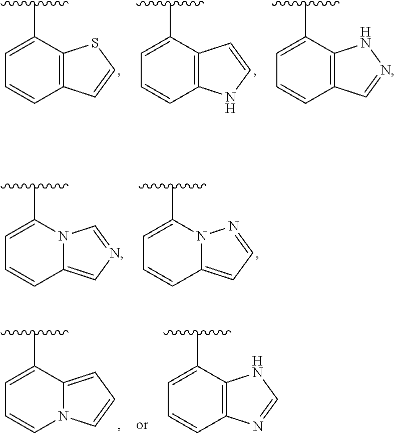

In particular embodiments, R³ is:

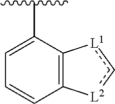

wherein:
L¹ and L² are independently selected from the group consisting of C, O, N and S.

In exemplary embodiments, L¹ is C and L² is selected from the group consisting of O, N and S; especially N or S. In alternative embodiments, L² is C and L¹ is selected from the group consisting of O, N and S; especially N or S. In particular embodiments, L¹ is C and L² is N, or L¹ is S and L² is C. In specific embodiments, R³ is:

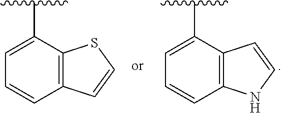 [SMILES: *c1cccc2ccsc12 ; *c1cccc2[nH]ccc12]

In some embodiments, X is N. In alternative embodiments, X is C.

In some embodiments, Y is N. In alternative embodiments, Y is C.

In some embodiments, Z is C or N; especially N.

In exemplary embodiments:
R¹ is selected from the group consisting of optionally substituted C₁₋₆ alkyl, —C(O)—NH₂, optionally substituted —C(O)—C₁₋₆ alkyl, —C(OH)—NH₂, —C(O)—OH, —CH₂—NH₂, —C(O)H and optionally substituted C₁₋₆ alkoxy;
R² is selected from the group consisting of H, ═O, hydroxyl and halogen;
R³ is selected from the group consisting of optionally substituted C₆₋₁₈ aryl and optionally substituted 5- to 18-membered heteroaryl;
X is N;
Y is N; and
Z is N.

In particular embodiments:
R¹ is selected from the group consisting of —C(O)—NH₂, optionally substituted —C(O)—C₁₋₆ alkyl, —C(OH)—NH₂ and —C(O)—OH;
R² is selected from the group consisting of H and ═O;
R³ is optionally substituted 5- to 18-membered heteroaryl;
X is N;
Y is N; and
Z is N.

In some embodiments:
R¹ is selected from the group consisting of —C(O)—NH₂ and optionally substituted —C(O)—C₁₋₆ alkyl;
R² is selected from the group consisting of H and ═O;
R³ is optionally substituted 5- to 18-membered heteroaryl;
X is N;
Y is N; and
Z is N.

In some embodiments:
R¹ is —C(O)—NH₂;
R² is selected from the group consisting of H and ═O;
R³ is optionally substituted 5- to 18-membered heteroaryl;
X is N;
Y is N; and
Z is N.

In some embodiments:
R¹ is —C(O)—NH₂;
R² is selected from the group consisting of H and ═O;
R³ is

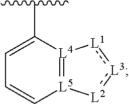

X is N;
Y is N;
Z is N;
L¹, L², L³, L⁴ and L⁵ are independently selected from the group consisting of C, O, N and S.

In some embodiments:
R¹ is —C(O)—NH₂;
R² is selected from the group consisting of H and ═O;
R³ is

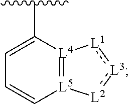

X is N;
Y is N;
Z is N;
one or two of L¹, L², L³, L⁴ and L⁵ are independently selected from the group consisting of O, N and S and the remainder are C.

In some embodiments:
R¹ is —C(O)—NH₂;
R² is selected from the group consisting of H and ═O;
R³ is

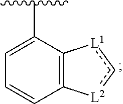

X is N;
Y is N;
Z is N;
L¹ is selected from the group consisting of C, O, N and S; and
L² is selected from the group consisting of C, O, N and S.

In some embodiments:
R¹ is —C(O)—NH₂;
R² is selected from the group consisting of H and ═O;
R³ is

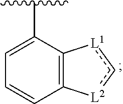

X is N;
Y is N;
Z is N;
L¹ is C or S; and
L² is C or N.

In particular embodiments, the compound of Formula II is a compound of Formula IV or V:

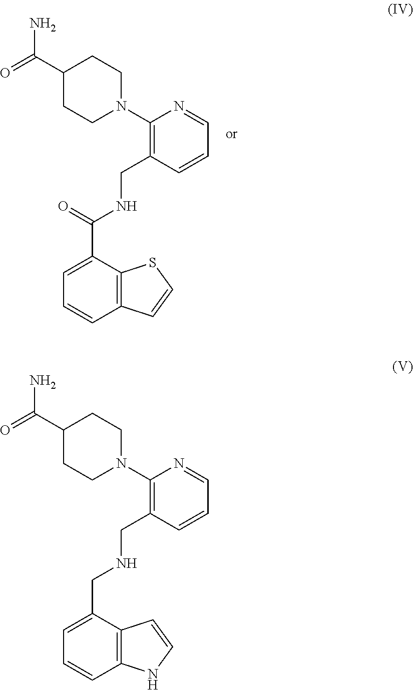 [SMILES: NC(=O)C1CCN(c2ncccc2CNC(=O)c2cccc3ccsc23)CC1 ; NC(=O)C1CCN(c2ncccc2CNCc2cccc3[nH]ccc23)CC1]

or a pharmaceutically acceptable salt thereof.

In particular embodiments, the compound of Formula II is a compound of Formula VI, VII, VIII, IX or X:

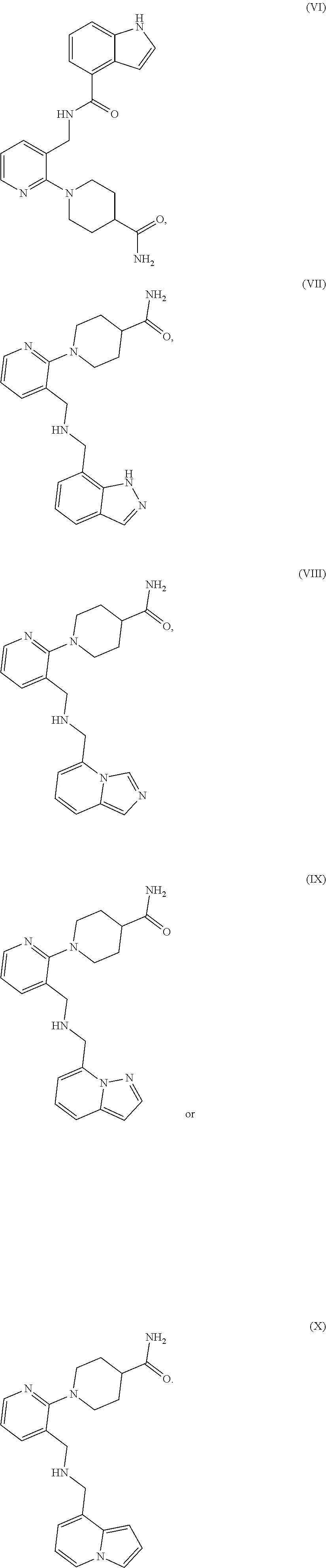 [SMILES: NC(=O)C1CCN(c2ncccc2CNC(=O)c2cccc3[nH]ccc23)CC1 ; NC(=O)C1CCN(c2ncccc2CNCc2cccc3cn[nH]c23)CC1 ; NC(=O)C1CCN(c2ncccc2CNCc2cccc3cncn23)CC1 ; NC(=O)C1CCN(c2ncccc2CNCc2cccc3ccnn23)CC1 ; NC(=O)C1CCN(c2ncccc2CNCc2cccn3cccc23)CC1]

In particular embodiments, the compound of Formula II is a compound of Formula IV, V, VI, VII, VIII, IX, X, XI, XV or XVI. In some embodiments, the compound of Formula II is a compound of Formula VI, VII, VIII, IX, X, XI, XV or XVI; especially a compound of Formula VI or XVI.

In some embodiments, the compound of the invention is a compound of Formula III or a pharmaceutically acceptable salt thereof:

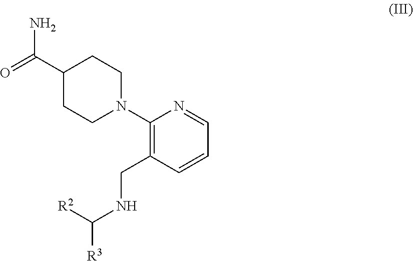

wherein R² and R³ are as described above for Formula I or II.

Suitable embodiments of R² and R³ are as discussed supra.

In some embodiments, the compound of the invention is a compound of Formula IIIA or a pharmaceutically acceptable salt thereof:

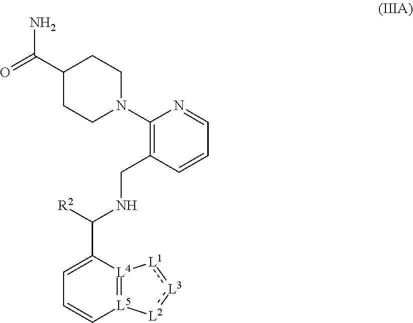

wherein L¹, L² L³, L⁴, L⁵ and R² are as described above for Formula I.

In some embodiments, the compound of the invention is a compound of Formula IIIB or a pharmaceutically acceptable salt thereof:

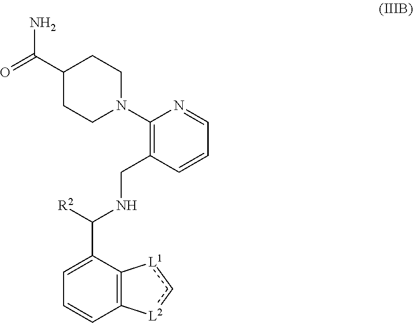

wherein L¹, L² and R² are as described above for Formula I.

Suitable embodiments of L¹, L² and R² are as discussed supra.

In particular embodiments, the compound of Formula I, IA, IB, II, III, IIIA or IIIB is compound 2 or compound 22 as defined in the examples. In particular embodiments, the compound of Formula I, IA, IB, II, III, IIIA or IIIB is any one of compounds 2, 22 and 31-40 as defined in the examples.

The compounds of the invention may be in neutral form or may be in the form of salts (especially pharmaceutically acceptable salts), prodrugs, solvates (including hydrates), solvates (including hydrates) of salts, esters and polymorphs. In preferred embodiments, such forms are pharmaceutically acceptable forms. The compounds of the invention, including the compounds of Formulae I-XVI, may be prepared in crystalline or non-crystalline form, and may be optionally hydrated or solvated. The invention includes stoichiometric hydrates as well as compounds containing variable amounts of water. Solvates include stoichiometric solvates and non-stoichiometric solvates.

It is to be understood that the present invention encompasses all isomers of the compounds of the invention, including the compounds of Formulae I-XVI and their pharmaceutically acceptable derivatives, including all geometric, tautomeric and optical forms, and mixtures thereof (e.g. racemic mixtures). Where additional chiral centres are present in the compounds of the invention, the present invention includes within its scope all possible diastereomers, including mixtures thereof. The different isomeric forms may be separated or resolved one from the other by conventional methods, or any given isomer may be obtained by conventional synthetic methods or by stereospecific or asymmetric syntheses.

The compounds of the invention may readily be prepared using standard organic chemistry techniques. For example, compounds of Formula I or II may be prepared using the following general scheme:

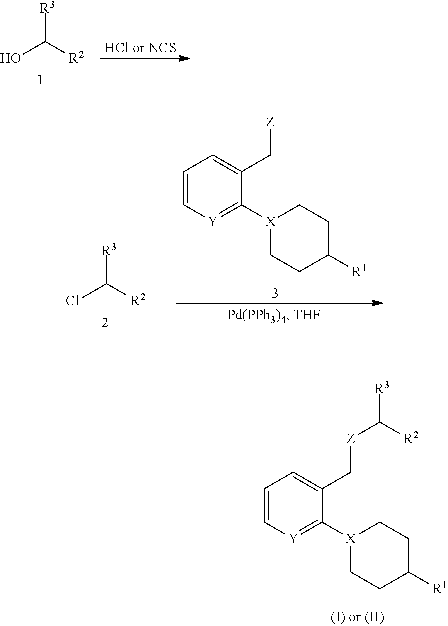

In brief, an R² and R³-containing precursor (1) is chlorinated using, for example, hydrochloric acid (HCl) or N-chlorosuccinimide (NCS). The chlorinated precursor (2) is then reacted with the R¹-containing precursor (3) in the presence of a palladium catalyst, such as Pd(PPh₃)₄, and tetrahydrofuran (THF) to generate a compound of Formula I or II. Alternatively, the chlorinated precursor (2) may be reacted with the R¹-containing precursor (3) in the presence of K₂CO₃ in dimethylformamide (DMF) to generate a compound of Formula I or II.

In exemplary embodiments, a compound of Formula V may be prepared using the following scheme:

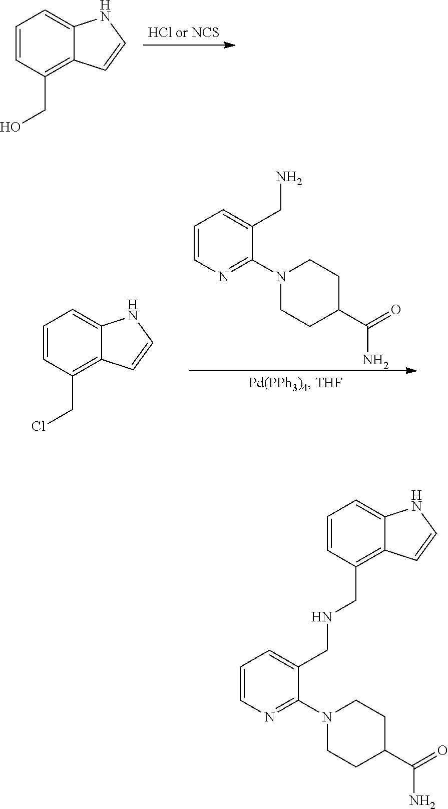 [SMILES: OCc1cccc2[nH]ccc12 ; ClCc1cccc2[nH]ccc12 ; NCc1cccnc1N1CCC(C(N)=O)CC1 ; NC(=O)C1CCN(c2ncccc2CNCc2cccc3[nH]ccc23)CC1]

A compound of Formula IV may be prepared using a similar scheme.

When R² is H and Z is N, compounds may be prepared using the following exemplary scheme:

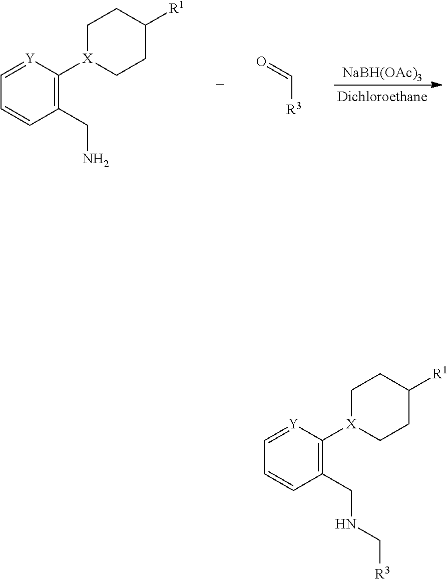

When R² is ═O and Z is N, compounds may be prepared using the following exemplary scheme:

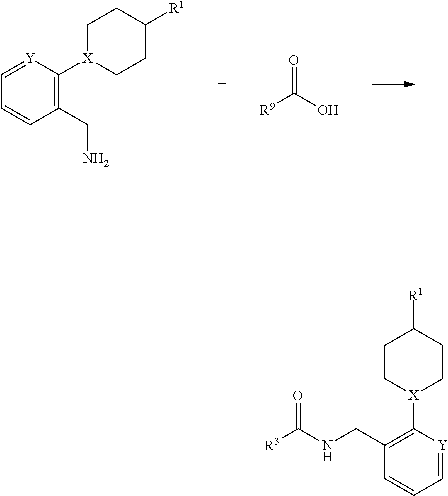

Amide bond formation between the two precursors may be achieved using a coupling reagent in a suitable solvent, such as 1-[bis(dimethylamino)methylene]-1H-1,2,3-triazolo[4,5-b]pyridinium 3-oxid hexafluorophosphate (HATU) and N,N-diisopropylethylamine (DIPEA) in acetonitrile, or benzotriazole-1-yl-oxy-tris-pyrrolidino-phosphonium hexafluorophosphate (PyBOP) and DIPEA in DMF.

When m is 1 and Z is N, compounds may be prepared using the following exemplary scheme:

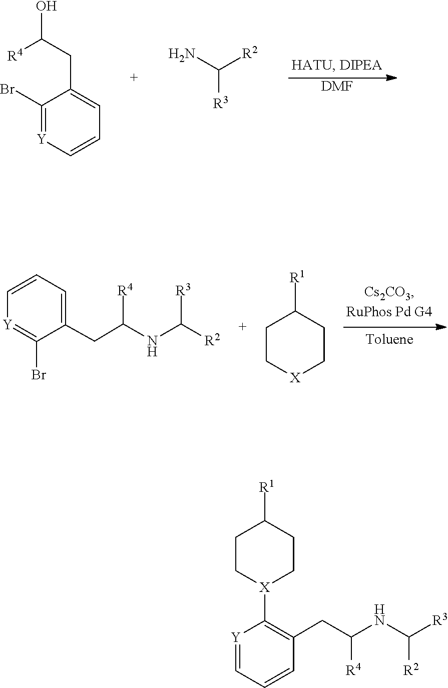

When R² and R³ together with the carbon atom to which they are attached form an optionally substituted C₆₋₁₈ aryl, optionally substituted 5- to 18-membered heteroaryl, optionally substituted C₅₋₁₈ cycloalkyl and optionally substituted 5- to 18-membered heterocycloalkyl, the compounds may be prepared using the following exemplary scheme (e.g. ring A in the below scheme):

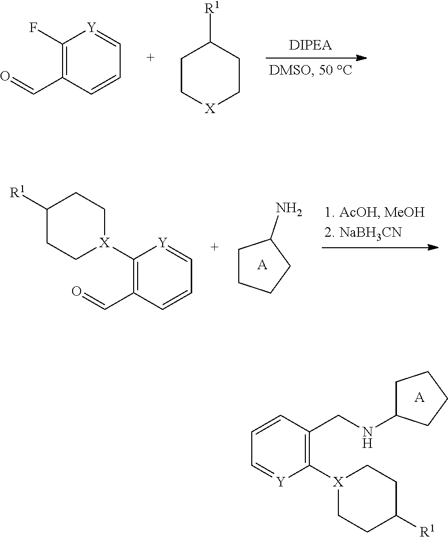

Starting materials and precursors are commercially available from a number of sources, including, for example, Sigma-Aldrich (St Louis, USA).

### 5. Compositions

In accordance with the present invention, the compounds of the invention are also useful in compositions and methods for treating and inhibiting the development of conditions associated with NSP5 activity, including a coronavirus infection or for treating or inhibiting the development of conditions associated with coronavirus infections. While it is possible that the compounds of the invention may be administered in an undiluted form, it is preferable to present such compounds in the form of a composition. Thus, in another aspect, there is provided a composition comprising a compound or a pharmaceutically acceptable salt thereof of the invention and a pharmaceutically acceptable carrier or diluent. In preferred embodiments, the composition is a pharmaceutical composition.

The compound may be formulated into the pharmaceutical composition as a neutral or salt form.

As will be appreciated by those skilled in the art, the choice of pharmaceutically acceptable carrier or diluent will be dependent on the route of administration and on the nature of the condition and subject to be treated. The particular carrier or delivery system and route of administration may be readily determined by a person skilled in the art. The carrier or delivery system and route of administration should be carefully selected to ensure that the activity of the compound is not depleted during preparation of the formulation and the compound is able to reach the site of action intact. The compositions of the invention may be administered through a variety of routes including, but not limited to, oral, rectal, topical, intranasal, inhalation, intraocular, transmucosal, intestinal, enteral, intramuscular, subcutaneous, intramedullary, intrathecal, intraventricular, intracerebral, intravaginal, intravesical, intravenous or intraperitoneal administration. Optionally, compositions of the invention may be administered by infusion, typically intravenous infusion. In particular embodiments, the pharmaceutical composition is administered orally, intranasally, by inhalation or by infusion.

The pharmaceutical forms suitable for injectable use include sterile injectable solutions or dispersions and sterile powders for the preparation of sterile injectable solutions. Such forms should be stable under the conditions of manufacture and storage and may be preserved against reduction, oxidation and microbial contamination.

A person skilled in the art will readily be able to determine appropriate formulations for the compounds using conventional approaches. Techniques for formulation and administration may be found in, for example, Remington: The Science and Practice of Pharmacy, Adeboye Adejare and Joseph Remington (Ed), Academic Press, London, 23^{rd} Edition, 2021.

Identification of preferred pH ranges and suitable excipients, such as antioxidants, is routine in the art, for example, as described in Katdare and Chaubel (2006) Excipient Development for Pharmaceutical, Biotechnology and Drug Delivery Systems (CRC Press). Buffer systems are routinely used to provide pH values of a desired range and may include, but are not limited to, carboxylic acid buffers, such as acetate, citrate, lactate, tartrate and succinate; glycine; histidine; phosphate; tris(hydroxymethyl)aminomethane (Tris); arginine; sodium hydroxide; glutamate; and carbonate buffers. Suitable antioxidants may include, but are not limited to, phenolic compounds such as butylated hydroxytoluene (BHT) and butylated hydroxyanisole; vitamin E; ascorbic acid; reducing agents such as methionine or sulphite; metal chelators such as ethylene diamine tetraacetic acid (EDTA); cysteine hydrochloride; sodium bisulfite; sodium metabisulfite; sodium sulfite; ascorbyl palmitate; lecithin; propyl gallate; and alpha-tocopherol.

For injection, the compound may be formulated in an aqueous solution, suitably in a physiologically compatible buffer such as Hanks' solution, Ringer's solution, dextrose solution or physiological saline buffer, such as phosphate buffered saline (PBS). For transmucosal administration, penetrants appropriate to the barrier to be permeated are used in the formulation. Such penetrants are generally known in the art.

The compositions of the present invention may be formulated for administration in the form of liquids, containing acceptable diluents (such as saline and sterile water), or may be in the form of lotions, creams or gels containing acceptable diluents or carriers to impart the desired texture, consistency, viscosity and appearance. Acceptable diluents and carriers are familiar to those skilled in the art and include, but are not restricted to, ethoxylated and nonethoxylated surfactants, fatty alcohols, fatty acids, hydrocarbon oils (such as palm oil, coconut oil, and mineral oil), cocoa butter waxes, silicon oils, pH balancers, cellulose derivatives, emulsifying agents such as non-ionic organic and inorganic bases, preserving agents, wax esters, steroid alcohols, triglyceride esters, phospholipids such as lecithin and cephalin, polyhydric alcohol esters, fatty alcohol esters, hydrophilic lanolin derivatives and hydrophilic beeswax derivatives.

Alternatively, the compound can be formulated readily using pharmaceutically acceptable carriers well known in the art into dosages suitable for oral administration, which is also contemplated for the practice of the invention. In some embodiments, the compound of the invention is formulated for oral administration in a dosage form such as a tablet, pill, capsule, liquid, gel, syrup, slurry, suspension, lozenge and the like for oral ingestion by a subject. In particular embodiments, the compound of the invention is formulated for oral administration in a solid dosage form, such as a tablet, pill, lozenge or capsule. In such embodiments, the pharmaceutically acceptable carrier may comprise a number of excipients including, but not limited to, a diluent, disintegrant, binder, lubricant, glidant and the like.

Suitable diluents (also referred to as “fillers”) include, but are not limited to, lactose (including lactose monohydrate, spray-dried monohydrate, anhydrous, etc.), mannitol, xylitol, dextrose, sucrose, sorbitol, compressible sugar, isomalt, microcrystalline cellulose, powdered cellulose, starch, pregelatinised starch, dextrates, dextran, dextrin, dextrose, maltodextrin, calcium carbonate, dibasic calcium phosphate, tribasic calcium phosphate, calcium sulfate, magnesium carbonate, magnesium oxide, poloxamers, polyethylene oxide, hydroxypropyl methyl cellulose, silicates (e.g. silicon dioxide), polyvinyl alcohol, talc, and combinations thereof.

Suitable disintegrants include, but are not limited to, sodium carboxymethyl cellulose, pregelatinised starch, calcium carboxymethyl cellulose, croscarmellose sodium, crospovidone, polyvinylpyrrolidone, methylcellulose, sodium starch glycolate, microcrystalline cellulose, lower alkyl-substituted hydroxypropyl cellulose, starch, sodium alginate and combinations thereof.

Suitable binders include, but are not limited to, microcrystalline cellulose, gelatine, sugars, polyethylene glycol, natural and synthetic gums, polyvinylpyrrolidone, pregelatinised starch, hydroxypropyl cellulose, hydroxypropyl methylcellulose and combinations thereof.

Suitable lubricants include, but are not limited to, magnesium stearate, calcium stearate, zinc stearate, sodium stearyl fumarate, polyethylene glycol and combinations thereof.

Suitable glidants include, but are not limited to, silicon dioxide, colloidal silicon dioxide, magnesium silicate, magnesium trisilicate, talc and combinations thereof.

The composition may also include a buffer and/or antioxidant as discussed supra. Further components of any one of the dosage forms discussed herein may include, but are not limited to, surfactants, flavouring agents, sweeteners, preservatives, and the like.

Pharmaceutical formulations for parenteral administration include aqueous solutions of the compound of the invention in water-soluble form. Additionally, suspensions of the compound may be prepared as appropriate oily injection suspensions. Suitable lipophilic solvents or vehicles include fatty oils such as sesame oil, or synthetic fatty acid esters, such as ethyl oleate or triglycerides. Aqueous injection suspensions may contain substances that increase the viscosity of the suspension, such as sodium carboxymethyl cellulose, sorbitol or dextran. Optionally, the suspension may also contain suitable stabilisers or agents that increase the solubility of the compounds to allow for the preparation of highly concentrated solutions.

Sterile solutions may be prepared by combining the compound in the required amount in the appropriate solvent with other excipients as described above as required, followed by sterilisation, such as filtration. Generally, dispersions are prepared by incorporating the various sterilised active compounds into a sterile vehicle which contains the basic dispersion medium and the required excipients as described above. Sterile dry powders may be prepared by vacuum- or freeze-drying a sterile solution comprising the active compounds and other required excipients as described above.

Pharmaceutical preparations for oral use can be obtained by combining the compounds with solid excipients and processing the mixture of granules, after adding suitable auxiliaries, if desired, to obtain tablets or dragee cores. Such compositions may be prepared by any of the methods of pharmacy but all methods include the step of bringing into association one or more compounds of the invention as described above with the carrier which constitutes one or more necessary ingredients. In general, the pharmaceutical compositions of the invention may be manufactured in a manner that is itself known, e.g. by means of conventional mixing, dissolving, granulating, dragee-making, levigating, emulsifying, encapsulating, entrapping or lyophilising processes.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterise different combinations of particle doses.

Pharmaceuticals which can be used orally include push-fit capsules made of gelatin, as well as soft, sealed capsules made of gelatin and a plasticiser, such as glycerol or sorbitol. The push-fit capsules can contain the active ingredients in admixture with a filler such as lactose, binders such as starches, and/or lubricants such as talc or magnesium stearate and, optionally, stabilisers. In soft capsules, the active compounds may be dissolved or suspended in suitable liquids, such as fatty oils, liquid paraffin, or liquid polyethylene glycols. In addition, stabilisers may be added.

The compounds of the invention may be incorporated into modified-release preparations and formulations, for example, polymeric microsphere formulations, and oil- or gel-based formulations.

The compounds may be administered in a local rather than systemic manner, such as by injection directly into a tissue, which is preferably subcutaneous or omental tissue, often in a depot or sustained release formulation. In other embodiments, the compound is systemically administered.

Furthermore, the compound may be administered in a targeted drug delivery system, such as in a particle which is suitable targeted to and taken up selectively by a cell or tissue. In some embodiments, the compound is contained or otherwise associated with a vehicle selected from liposomes, micelles, dendrimers, biodegradable particles, artificial DNA nanostructure, lipid-based nanoparticles and carbon or old nanoparticles. In illustrative examples of this type, the vehicle is selected from poly(lactic acid) (PLA), poly(glycolic acid) (PGA), poly(lactic-co-glycolic acid) (PLGA), poly(ethylene glycol) (PEG), PLA-PEG copolymers and combinations thereof.

In cases of local administration or selective uptake, the effective local concentration of the agent may not be related to plasma concentration.

In some embodiments, the compositions are suitable for inhalation or intranasal delivery and are in the form of, for example, solutions, aerosols, dry powders, suspensions or emulsions. For example, the composition may be administered to the respiratory tract as a nasal or pulmonary inhalation aerosol or solution for a nebuliser, or as a microfine powder (e.g. with particles in the order of 1 to 10 μm in diameter or less) for insufflation, alone or in combination with an inert carrier, such as lactose or glucose, or with other pharmaceutically acceptable excipients, such as beta-cyclodextrin, starch, sodium carboxymethylcellulose and the like.

Aerosol formulations include those in which the compound is provided in a pressurised pack with a suitable propellant such as a pressurised metered dose inhaler (pMDI). Whilst the propellant may be a chlorofluorocarbon (CFC) such as dichlorodifluoromethane, trichlorofluoromethane, dichlorotetrafluoroethane, the propellant is more preferably a non-chlorofluorocarbon propellant such as carbon dioxide, hydrofluoroalkanes (such as HFA-134a) or another suitable gas. The aerosol may conveniently also contain a surfactant such as lecithin. The dose of the compound may be controlled by provision of a metered valve. For delivery to the lung, a particle size of approximately 1 to 5 μm is useful, as particles smaller than 1 μm are generally exhaled without delivery to the lung and particles larger than 10 μm are mostly trapped by oropharyngeal deposition and do not reach the lung. Devices propelled by HFA-134a deliver smaller droplets which penetrate more readily into the bronchial airways. For drug delivery via the nasal passage, a suitable particle size is, for example, 20-80 μm, as smaller particles (less than 10 μm) get carried into the tracheobrachial region, whilst bigger particles (greater than 100 μm) get rapidly cleared from the nasal passageway.

The compound may also be provided in a pharmaceutical formulation which forms a gel in the nasal cavity. The compound may, alternatively, be formulated in a powder composition which may be presented in unit dose form for example in capsules or cartridges of e.g. gelatin, or blister packs from which the powder may be administered by means of an inhaler.

It is advantageous to formulate the compositions in dosage unit form for ease of administration and uniformity of dosage. The determination of the novel dosage unit forms of the present invention is dictated by and directly dependent on the unique characteristics of the active material, the particular therapeutic effect to be achieved and the limitations inherent in the art of compounding active materials for the treatment of disease in living subjects having a diseased condition in which bodily health is impaired as herein disclosed in detail.

While a compound of the invention may be the sole active agent administered to the subject, the administration of other active agents concurrently with said compound is within the scope of the invention. For example, in some embodiments, the compound may be administered concurrently with one or more antimicrobial agents, anti-inflammatory agents, or agents which inhibit the cytokine storm. The compound may be therapeutically used after the other active agent or may be therapeutically used together with the other active agent. The compound may be administered separately, simultaneously or sequentially with the other active agent.

Accordingly, in another aspect of the invention, there is provided a composition comprising a compound of the invention and an antiviral agent, an anti-inflammatory agent or an agent which inhibits the cytokine storm.

Representative antivirals include abacavir sulfate, acyclovir sodium, adefovir, amantadine hydrochloride, amprenavir, atazanavir, baloxavir marboxil, bictegravir, boceprevir, bulevirtide, cidofovir, cobicistat, daclatasvir, darunavir, delavirdine mesylate, didanosine, docosanol, dolutegravir, doravinine, edoxudine, efavirenz, elvitegravir, emtricitabine, enfuvirtide, ensitrelvir, entecavir, etravirine, famciclovir, fomivirsen sodium, fosamprenavir, foscarnet sodium, ganciclovir, ibacitabine, ibalizumab, idoxuridine, imiquimod, inosine pranobex, indinavir sulfate, lamivudine, lamivudine/zidovudine, letermovir, lopinavir, loviride, maraviroc, methisazone, molnupiravir, moroxydine, nelfinavir mesylate, nirmatrelvir, nirmatrelvir and ritonavir, nevirapine, nitazoxanide, norvir, oseltamivir phosphate, penciclovir, peramivir, pleconaril, polophyllotoxin, raltegravir, remdesivir, ribavirin, rilpivarine, rimantadine hydrochloride, ritonavir, saquinavir, saquinavir mesylate, simeprevir, sofosbuvir, stavudine, taribavirin, telaprevir, telbivudine, tenofovir disoproxil, tenofovir alafenamide, tipranavir, trifluridine, trizivir, tromantadine, truvada, umifenovir, valacyclovir, valacyclovir hydrochloride, valganciclovir, zalcitabine, zanamivir, zidovudine and mixtures thereof.

In some embodiments, the ancillary active agent is an anti-inflammatory agent, representative examples of which include steroidal anti-inflammatory agents such as but not limited to compounds containing a 17-carbon 4-ring system, including sterols, various hormones (as anabolic steroids), and glycosides. Representative examples of steroidal anti-inflammatory drugs include, without limitation, corticosteroids such as hydrocortisone, hydroxyltriamcinolone, alpha-methyl dexamethasone, dexamethasone-phosphate, beclomethasone dipropionates, clobetasol valerate, desonide, desoxymethasone, desoxycorticosterone acetate, dexamethasone, dichlorisone, diflucortolone valerate, fluadrenolone, fluclorolone acetonide, flumethasone pivalate, fluosinolone acetonide, fluocinonide, flucortine butylesters, fluocortolone, fluprednidene (fluprednylidene) acetate, flurandrenolone, halcinonide, hydrocortisone acetate, hydrocortisone butyrate, methylprednisolone, triamcinolone acetonide, cortisone, cortodoxone, flucetonide, fludrocortisone, difluorosone diacetate, fluradrenolone, fludrocortisone, diflorosone diacetate, fluradrenolone acetonide, medrysone, amcinafel, amcinafide, betamethasone and the balance of its esters, chloroprednisone, chlorprednisone acetate, clocortelone, clescinolone, dichlorisone, diflurprednate, flucloronide, flunisolide, fluoromethalone, fluperolone, fluprednisolone, hydrocortisone valerate, hydrocortisone cyclopentylpropionate, hydrocortamate, meprednisone, paramethasone, prednisolone, prednisone, beclomethasone dipropionate, triamcinolone and mixtures thereof.

Alternatively, the anti-inflammatory agent may be a nonsteroidal anti-inflammatory agent, non-limiting examples of which include agents that are aspirin-like in their action, including, but not limited to, ibuprofen (ADVIL), naproxen sodium (ALEVE), and acetaminophen (TYLENOL). Additional examples of non-steroidal anti-inflammatory agents include, without limitation, oxicams, such as piroxicam, isoxicam, tenoxicam, sudoxicam, and CP-14,304; disalcid, benorylate, trilisate, safapryn, solprin, diflunisal, and fendosal; acetic acid derivatives, such as diclofenac, fenclofenac, indomethacin, sulindac, tolmetin, isoxepac, furofenac, tiopinac, zidometacin, acematacin, fentiazac, zomepirac, clindanac, oxepinac, felbinac, and ketorolac; fenamates, such as mefenamic, meclofenamic, flufenamic, niflumic, and tolfenamic acids; propionic acid derivatives, such as benoxaprofen, flurbiprofen, ketoprofen, fenoprofen, fenbufen, indopropfen, pirprofen, carprofen, oxaprozin, pranoprofen, miroprofen, tioxaprofen, suprofen, alminoprofen, and tiaprofenic; pyrazoles, such as phenylbutazone, oxyphenbutazone, feprazone, azapropazone, and trimethazone. Mixtures of these non-steroidal anti-inflammatory agents also may be employed, as well as the dermatologically acceptable salts and esters of these agents. For example, etofenamate, a flufenamic acid derivative, is particularly useful for topical application.

In other embodiments, the anti-inflammatory agent includes, without limitation, transforming growth factor-beta 3 (TGF-β), an anti-tumour necrosis factor-alpha (TNF-α) agent, an inhibitor or antagonist of IL-6 or IL-6 receptor, IL-1 receptor, IL-1β, TNF, GM-CSF, IFN-γ, JAK-STAT signalling, CCR2, CCR5, complement component C5, IRAK4 and M-CSF receptor, or a combination thereof.

The compounds of the invention may also be administered concurrently with agents which inhibit the cytokine storm. For example, suitable agents include compounds that target fundamental immune pathways, such as the chemokine network and the cholinergic anti-inflammatory pathway. For example, JAK inhibitors, such as JAK 1 and JAK 2 inhibitors, can inhibit the cytokine storm, and in some cases, are also antiviral. Representative JAK inhibitors include those disclosed in U.S. Pat. No. 10,022,378, such as Jakafi, Tofacitinib, and Baricitinib, as well as LY3009104/INCB28050, Pacritinib/SB1518, VX-509, GLPG0634, INC424, R-348, CYT387, TG 10138, AEG 3482, and pharmaceutically acceptable salts and prodrugs thereof. Still further examples include CEP-701 (Lestaurtinib), AZD1480, INC424, R-348, CYT387, TG 10138, AEG 3482, 7-iodo-N-(4-morpholinophenyl)thieno[3,2-d]pyrimidin-2-amine, 7-(4-aminophenyl)-N-(4-morpholinophenyl)thieno[3,2-d]pyrimidin-2-amine, N-(4-(2-(4-morpholinophenylamino)thieno[3,2-d]pyrimidin-7-yl)phenyl) acrylamide, 7-(3-aminophenyl)-N-(4-morpholinophenyl)thieno[3,2-d]pyrimidin-2-amine, N-(3-(2-(4-morpholinophenylamino)thieno[3,2-d]pyrimidin-7-yl)phenyl) acrylamide, N-(4-morpholinophenyl)thieno[3,2-d]pyrimidin-2-amine, methyl 2-(4-morpholinophenylamino)thieno[3,2-d]pyrimidine-7-carboxylate, N-(4-morpholinophenyl)-5H-pyrrolo[3,2-d]pyrimidin-2-amine, 7-(4-amino-3-methoxyphenyl)-N-(4-morpholinophenyl)thieno[3,2-d]pyrimidin-2-amine, 4-(2-(4-morpholinophenylamino)thieno[3,2-d]pyrimidin-7-yl)benzenesulfonamide, N,N-dimethyl-3-(2-(4-morpholinophenylamino)thieno[3,2-d]pyrimidin-7-yl)benzenesulfonamide, 1-ethyl-3-(2-methoxy-4-(2-(4-morpholinophenylamino)thieno[3,2-d]pyrimidin- -7-yl)phenyl)urea, N-(4-(2-(4-morpholinophenylamino)thieno[3,2-d]pyrimidin-7-yl)phenyl)methanesulfonamide, 2-methoxy-4-(2-(4-morpholinophenylamino)thieno[3,2-d]pyrimidin-7-yl)phenol, 2-cyano-N-(3-(2-(4-morpholinophenylamino)thieno[3,2-d]pyrimidin-7-yl)phenyl)acetamide, N-(cyanomethyl)-2-(4-morpholinophenylamino)thieno[3,2-d]pyrimidine-7-carboxamide, N-(3-(2-(4-morpholinophenylamino)thieno[3,2-d]pyrimidin-7-yl)phenyl)methanesulfonamide, 1-ethyl-3-(4-(2-(4-morpholinophenylamino)thieno[3,2-d]pyrimidin-7-yl)-2-(trifluoromethoxy)phenyl)urea, N-(3-nitrophenyl)-7-phenylthieno[3,2-d]pyrimidin-2-amine, 7-iodo-N-(3-nitrophenyl)thieno[3,2-d]pyrimidin-2-amine, N1-(7-(2-ethylphenyl)thieno[3,2-d]pyrimidin-2-yl)benzene-1,3-diamine, N-tert-butyl-3-(2-(4-morpholinophenylamino)thieno[3,2-d]pyrimidin-7-yl)benzenesulfonamide, N1-(7-iodothieno[3,2-d]pyrimidin-2-yl)benzene-1,3-diamine, 7-(4-amino-3-(trifluoromethoxy)phenyl)-N-(4-morpholinophenyl)thieno[3,2-d]pyrimidin-2-amine, 7-(2-ethylphenyl)-N-(4-morpholinophenyl)thieno[3,2-d]pyrimidin-2-amine, N-(3-(2-(4-morpholinophenylamino)thieno[3,2-d]pyrimidin-7-yl)phenyl)acetamide, N-(cyanomethyl)-N-(3-(2-(4-morpholinophenylamino)thieno[3,2-d]pyrimidin-7-yl)phenyl)methanesulfonamide, N-(cyanomethyl)-N-(4-(2-(4-morpholinophenylamino)thieno[3,2-d]pyrimidin-7-yl)phenyl)methanesulfonamide, N-(3-(5-methyl-2-(4-morpholinophenylamino)-5H-pyrrolo[3,2-d]pyrimidin-7-yl)phenyl)methanesulfonamide, 4-(5-methyl-2-(4-morpholinophenylamino)-5H-pyrrolo[3,2-d]pyrimidin-7-yl)benzenesulfonamide, N-(4-(5-methyl-2-(4-morpholinophenylamino)-5H-pyrrolo[3,2-d]pyrimidin-7-yl)phenyl)methanesulfonamide, 7-iodo-N-(4-morpholinophenyl)-5H-pyrrolo[3,2-d]pyrimidin-2-amine, 7-(2-isopropylphenyl)-N-(4-morpholinophenyl)thieno[3,2-d]pyrimidin-2-amine, 7-bromo-N-(4-morpholinophenyl)thieno[3,2-d]pyrimidin-2-amine, N7-(2-isopropylphenyl)-N2-(4-morpholinophenyl)thieno[3,2-d]pyrimidine-2,7-diamine, N7-(4-isopropylphenyl)-N2-(4-morpholinophenyl)thieno[3,2-d]pyrimidine-2,7-diamine, 7-(5-amino-2-methylphenyl)-N-(4-morpholinophenyl)thieno[3,2-d]pyrimidin-2-amine, N-(cyanomethyl)-4-(2-(4-morpholinophenylamino)thieno[3,2-d]pyrimidin-7-yl)benzamide, 7-iodo-N-(3-morpholinophenyl)thieno[3,2-d]pyrimidin-2-amine, 7-(4-amino-3-nitrophenyl)-N-(4-morpholinophenyl)thieno[3,2-d]pyrimidin-2-amine, 7-(2-methoxypyridin-3-yl)-N-(4-morpholinophenyl)thieno[3,2-d]pyrimidin-2-amine, (3-(7-iodothieno[3,2-d]pyrimidin-2-ylamino)phenyl)methanol, N-tert-butyl-3-(2-(3-morpholinophenylamino)thieno[3,2-d]pyrimidin-7-yl)benzenesulfonamide, N-tert-butyl-3-(2-(3-(hydroxymethyl)phenylamino)thieno[3,2-d]pyrimidin-7-yl)benzenesulfonamide, N-(4-morpholinophenyl)-7-(4-nitrophenylthio)-5H-pyrrolo[3,2-d]pyrimidin-2-amine, N-tert-butyl-3-(2-(3,4,5-trimethoxyphenylamino)thieno[3,2-d]pyrimidin-7-yl)benzenesulfonamide, 7-(4-amino-3-nitrophenyl)-N-(3,4-dimethoxyphenyl)thieno[3,2-d]pyrimidin-2-amine, N-(3,4-dimethoxyphenyl)-7-(2-methoxypyridin-3-yl)thieno[3,2-d]pyrimidin-2-amine, N-tert-butyl-3-(2-(3,4-dimethoxyphenylamino)thieno[3,2-d]pyrimidin-7-yl)benzenesulfonamide, 7-(2-aminopyrimidin-5-yl)-N-(3,4-dimethoxyphenyl)thieno[3,2-d]pyrimidin-2-amine, N-(3,4-dimethoxyphenyl)-7-(2,6-dimethoxypyridin-3-yl)thieno[3,2-d]-pyrimidin-2-amine, N-(3,4-dimethoxyphenyl)-7-(2,4-dimethoxypyrimidin-5-yl)thieno[3,2-d]pyrimidin-2-amine, 7-iodo-N-(4-(morpholinomethyl)phenyl)thieno[3,2-d]pyrimidin-2-amine, N-tert-butyl-3-(2-(4-(morpholinomethyl)phenylamino)thieno[3,2-d]pyrimidin-7-yl)benzenesulfonamide, 2-cyano-N-(4-methyl-3-(2-(4-morpholinophenylamino)thieno[3,2-d]pyrimidin-7-yl)phenyl)acetamide, ethyl 3-(2-(4-morpholinophenylamino)thieno[3,2-d]pyrimidin-7-yl)benzoate, 7-bromo-N-(4-(2-(pyrrolidin-1-yl)ethoxy)phenyl)thieno[3,2-d]pyrimidin-2-amine, N-(3-(2-(4-(2-(pyrrolidin-1-yl)ethoxy)phenylamino)thieno[3,2-d]pyrimidin-7-yl)phenyl)acetamide, N-(cyanomethyl)-3-(2-(4-morpholinophenylamino)thieno[3,2-d]pyrimidin-7-yl)benzamide, N-tert-butyl-3-(2-(4-morpholinophenylamino)thieno[3,2-d]pyrimidin-7-yl)benzamide, N-tert-butyl-3-(2-(4-(1-ethylpiperidin-4-yloxy)phenylamino)thieno-[3,2-d]pyrimidin-7-yl)benzenesulfonamide, tert-butyl-4-(2-(4-(morpholinomethyl)phenylamino)thieno[3,2-d]pyrimidin-7-yl)-1H-pyrazole-1-carboxylate, 7-bromo-N-(4-((4-ethylpiperazin-1-yl)methyl)phenyl)thieno[3,2-d]pyrimidin-2-amine, N-tert-butyl-3-(2-(4-((4-ethylpiperazin-1-yl)methyl)phenylamino)-thieno[3,2-d]pyrimidin-7-yl)benzenesulfonamide, N-(4-((4-ethylpiperazin-1-yl)methyl)phenyl)-7-(1H-pyrazol-4-yl)thieno[3,2-d]pyrimidin-2-amine, N-(cyanomethyl)-3-(2-(4-(morpholinomethyl)phenylamino)thieno[3,2-d]pyrimidin-7-yl)benzamide, N-tert-butyl-3-(2-(4-(2-(pyrrolidin-1-yl)ethoxy)phenylamino)thieno[3,2-d]-pyrimidin-7-yl)benzenesulfonamide, tert-butyl pyrrolidin-1-yl)ethoxy)phenylamino)thieno[3,2-d]pyrimidin-7-yl)benzylcarbamate, 3-(2-(4-(2-(pyrrolidin-1-yl)ethoxy)phenylamino)thieno[3,2-d]pyrimidin-7-yl)benzenesulfonamide, 7-(3-chloro-4-fluorophenyl)-N-(4-(2-(pyrrolidin-1-yl)ethoxy)phenyl)thieno-[3,2-d]pyrimidin-2-amine, tert-butyl 4-(2-(4-(1-ethylpiperidin-4-yloxy)phenylamino)thieno[3,2-d]pyrimidin-7-yl)-1H-pyrazole-1-carboxylate, 7(benzo[d][1,3]dioxol-5-yl)-N-(4-(morpholinomethyl)phenyl)thieno[3,2-d]pyrimidin-2-amine, tert-butyl 5-(2-(4-(morpholinomethyl)phenylamino)thieno[3,2-d]pyrimidin-7-yl)-1H-indole-1-carboxylate, 7-(2-aminopyrimidin-5-yl)-N-(4-(morpholinomethyl)phenyl)thieno[3,2-d]pyrimidin-2-amine, tert-butyl 4-(2-(-4-(morpholinomethyl)phenylamino)thieno[3,2-d]pyrimidin-7-yl)-5,6-di-hydropyridine-1(2H)-carboxylate, tert-butyl morpholinomethyl)phenylamino)thieno[3,2-d]pyrimidin-7-yl)benzylcarbamate, N-(3-(2-(4-(morpholinomethyl)phenylamino)thieno[3,2-d]pyrimidin-7-yl)phenyl)acetamide, N-(4-(2-(4-(morpholinomethyl)phenylamino)thieno[3,2-d]pyrimidin-7-yl)phenyl)acetamide, N-(3-(2-(4-(morpholinomethyl)phenylamino)thieno[3,2-d]pyrimidin-7-yl)phenyl)methanesulfonamide, 7-(4-(4-methylpiperazin-1-yl)phenyl)-N-(4-(morpholinomethyl)phenyl)thieno-[3,2-d]pyrimidin-2-amine, N-(2-methoxy-4-(2-(4-(morpholinomethyl)phenylamino)thieno[3,2-d]pyrimidin-7-yl)phenyl)acetamide, 7-bromo-N-(3,4,5-trimethoxyphenyl)thieno[3,2-d]pyrimidin-2-amine, (3-(2-(3,4,5-trimethoxyphenylamino)thieno[3,2-d]pyrimidin-7-yl)phenyl)methanol, (4-(2-(3,4,5-trimethoxyphenylamino)thieno[3,2-d]pyrimidin-7-yl)phenyl)methanol, (3-(2-(4-morpholinophenylamino)thieno[3,2-d]pyrimidin-7-yl)phenyl)methanol, (4-(2-(4-morpholinophenylamino)thieno[3,2-d]pyrimidin-7-yl)phenyl)methanol, N-(pyrrolidin-1-yl)ethoxy)phenylamino)thieno[3,2-d]pyrimidin-7-yl)benzyl)methanesulfonamide, tert-butyl morpholinomethyl)phenylamino)thieno[3,2-d]pyrimidin-7-yl)benzylcarbamate, N-(4-(morpholinomethyl)phenyl)-7-(3-(piperazin-1-yl)phenyl)thieno[3,2-d]pyrimidin-2-amine, 7-(6-(2-morpholinoethylamino)pyridin-3-yl)-N-(3,4,5-trimethoxyphenyl)thieno[3,2-d]pyrimidin-2-amine, 7-(2-ethylphenyl)-N-(4-(2-(pyrrolidin-1-yl)ethoxy)phenyl)thieno[3,2-d]pyrimidin-2-amine, 7-(4-(aminomethyl)phenyl)-N-(4-(morpholinomethyl)phenyl)thieno[3,2-d]pyrimidin-2-amine, N-(4-(1-ethylpiperidin-4-yloxy)phenyl)-7-(1H-pyrazol-4-yl)thieno[3,2-d]pyrimidin-2-amine, N-(2,4-dimethoxyphenyl)-7-phenylthieno[3,2-d]pyrimidin-2-amine, 7-bromo-N-(3,4-dimethoxyphenyl)thieno[3,2-d]pyrimidin-2-amine, N-(3,4-dimethoxyphenyl)-7-phenylthieno[3,2-d]pyrimidin-2-amine, and pharmaceutically acceptable salts and prodrugs thereof.

Alternatively, or in addition, HMGB1 antibodies and/or COX-2 inhibitors can be used, which downregulate the cytokine storm. Examples of such compounds include Actemra (Roche) and Celebrex (celecoxib), a COX-2 inhibitor. IL-8 (CXCL8) inhibitors can also be used.

As previously described, the compound may be compounded for convenient and effective administration in effective amounts with a suitable pharmaceutically acceptable carrier in dosage unit form. Suitable unit dosages and maximum daily dosages of the compound of the invention may be determined in accordance with the unit doses and maximum daily doses used conventionally. In some embodiments, a unit dosage form may comprise the compound in an amount in the range of from about 0.25 μg to about 2000 mg. The compound may be present in an amount of from about 0.25 μg to about 2000 mg/mL of carrier. In embodiments where the pharmaceutical composition comprises one or more additional active ingredients, the dosages are determined by reference to the usual dose and manner of administration of the said ingredients.

### 6. Methods and Uses

The compounds of the invention have been found to inhibit NSP5 activity. Accordingly, the inventors have conceived that such compounds will be useful for: treating and inhibiting the development of conditions associated with NSP5 activity, including a coronavirus infection; treating or inhibiting the development of conditions associated with coronavirus infections; treating or inhibiting the development of neurological symptoms of, and/or neurological complications or impairments associated with, a coronavirus infection; and treating or inhibiting the development or progression of long COVID following SARS-CoV-2 infection. Therefore, a compound of the invention for use in therapy is contemplated.

Provided herein are methods of inhibiting NSP5 activity, comprising, consisting or consisting essentially of contacting NSP5 with a compound of the invention. Also provided is a compound of the invention for use in inhibiting NSP5 activity, a use of a compound of the invention for inhibiting NSP5 activity, and a use of a compound of the invention in the manufacture of a medicament for inhibiting NSP5 activity.

In some embodiments, the methods and uses involve inhibiting NSP5 activity in a subject, such as a subject with a viral infection or who is suspected of having a viral infection.

In particular embodiments, the NSP5 is coronavirus NSP5, such as the coronaviruses discussed elsewhere herein.

One or more activities of NSP5 may be inhibited, such as the enzymatic activity, including the proteolytic activity. In this regard, inhibiting one or more activities of NSP5 may include or effect the inhibition of cleavage of endogenous host cellular proteins, inhibition of assembly of the viral replication transcription complex and/or inhibition of the activity of downstream protease-dependent proteins.

Suitable embodiments of the compounds of the invention are discussed supra.

Also provided herein are methods of treating or inhibiting the development of a coronavirus infection in a subject, comprising, consisting or consisting essentially of administering a compound of the invention to the subject. Further contemplated is a compound of the invention for use in treating or inhibiting the development of a coronavirus infection in a subject, a use of a compound of the invention for treating or inhibiting the development of a coronavirus infection in a subject, and a use of a compound of the invention in the manufacture of a medicament for treating or inhibiting the development of a coronavirus infection in a subject.

In some embodiments, the coronavirus is capable of causing SARS.

In some embodiments, the coronavirus is a betacoronavirus, such as one selected from a lineage A betacoronavirus, a lineage B betacoronavirus, a lineage C betacoronavirus and a lineage D betacoronavirus. In some embodiments, the betacoronavirus is a lineage B betacoronavirus, such as SARS-CoV or SARS-CoV-2; especially SARS-CoV-2. In alternative embodiments, the betacoronavirus is a lineage C betacoronavirus, such as MERS-CoV.

In particular embodiments, the coronavirus is SARS-CoV-2.

Also provided herein are methods of treating or inhibiting the development of one or more neurological symptoms of a coronavirus infection in a subject or for treating or inhibiting the development of one or more neurological complications or impairments associated with a coronavirus infection in a subject, comprising, consisting or consisting essentially of administering a compound of the invention to the subject. Also provided is a compound of the invention for use in treating or inhibiting the development of one or more neurological symptoms of a coronavirus infection, or one or more neurological complications or impairments associated with a coronavirus infection, in a subject, a use of a compound of the invention for treating or inhibiting the development of one or more neurological symptoms of a coronavirus infection, or one or more neurological complications or impairments associated with a coronavirus infection, in a subject, and a use of a compound of the invention in the manufacture of a medicament for treating or inhibiting the development of one or more neurological symptoms of a coronavirus infection, or one or more neurological complications or impairments associated with a coronavirus infection, in a subject.

In particular embodiments, the coronavirus is a betacoronavirus, such as a lineage A betacoronavirus, a lineage B betacoronavirus, a lineage C betacoronavirus or a lineage D betacoronavirus. In some embodiments, the betacoronavirus is a lineage B betacoronavirus, such as SARS-CoV or SARS-CoV-2; especially SARS-CoV-2. In alternative embodiments, the betacoronavirus is a lineage C betacoronavirus, such as MERS-CoV. In specific embodiments, the coronavirus is SARS-CoV-2.

The neurological symptoms, complications or impairments may include, but are not limited to, headache, fatigue, dizziness, difficulty sleeping, anxiety, depression, impaired or loss of smell (anosmia), impaired or loss of taste (dysgeusia), seizure, stroke, muscular weakness, delirium, pain, psychosis, brain inflammation, Guillain-Barré syndrome and the like. The neurological symptoms, complications or impairments may comprise one or more cognitive challenges and/or deficits, such as loss of memory, impaired concentration, poor attention, confusion difficulties with thinking and difficulties with decision making.

While these neurological symptoms, complications or impairments may solely be present during and/or shortly after recovery from an acute infection, in some embodiments, the neurological symptoms are present following recovery from an acute infection and can be present for weeks, months or years following recovery and following resolution of the viral infection.

Accordingly, also provided herein are methods for treating or inhibiting the development or progression of long COVID or one or more symptoms thereof (also referred to as post COVID syndrome) in a subject following SARS-CoV-2 infection, comprising, consisting or consisting essentially of administering a compound or a pharmaceutically acceptable salt thereof of the invention to the subject. Also provided is a compound of the invention for use in treating or inhibiting the development or progression of long COVID or one or more symptoms thereof, a use of a compound of the invention for treating or inhibiting the development or progression of long COVID or one or more symptoms thereof, and a use of a compound of the invention in the manufacture of a medicament for treating or inhibiting the development or progression of long COVID or one or more symptoms thereof.

The symptoms or manifestations of long COVID may include, for example, fatigue, heart symptoms or conditions (such as myocarditis), digestive symptoms including diarrhea and stomach pain, joint or muscle pain, and one or more neurological symptoms, complications or impairments. The neurological symptoms, complications or impairments may include, for example, headache, fatigue, dizziness, difficulty sleeping, anxiety, depression, impaired or loss of smell (anosmia), impaired or loss of taste (dysgeusia), and cognitive challenges and/or deficits such as loss of memory, impaired concentration, poor attention, confusion difficulties with thinking and difficulties with decision making.

Inhibiting the progression of long COVID or one or more symptoms thereof may comprise severity and/or duration of the long COVID or symptom(s).

Also provided herein are methods of treating an acute inflammatory condition in a subject, wherein the condition is associated with a coronavirus infection, comprising, consisting or consisting essentially of administering a compound of the invention to the subject. Further provided is a compound of the invention for use in treating an acute inflammatory condition in a subject, wherein the condition is associated with a coronavirus infection, a use of a compound of the invention for treating an acute inflammatory condition in a subject, wherein the condition is associated with a coronavirus infection, and a use of a compound of the invention in the manufacture of a medicament for treating an acute inflammatory condition in a subject, wherein the condition is associated with a coronavirus infection.

In particular embodiments, the coronavirus is a betacoronavirus, such as a lineage A betacoronavirus, a lineage B betacoronavirus, a lineage C betacoronavirus or a lineage D betacoronavirus. In some embodiments, the betacoronavirus is a lineage B betacoronavirus, such as SARS-CoV or SARS-CoV-2; especially SARS-CoV-2. In alternative embodiments, the betacoronavirus is a lineage C betacoronavirus, such as MERS-CoV. In specific embodiments, the coronavirus is SARS-CoV-2.

Suitable embodiments of the compounds are discussed supra.

In some embodiments, the acute inflammatory condition is associated with presence of CRS or a cytokine storm. For example, this may include wherein the CRS or cytokine storm comprises an elevation of at least 50% compared to basal state of one or more cytokines selected from IFN-γ, IFN-β, TNF-α, IL-1β, IL-6, IL-17A, CCL3 and CXCL2.

The acute inflammatory condition may, for example, be associated with the presence of CRS, in which the subject has one or more symptoms selected from fever, fatigue, loss of appetite, muscle and joint pain, nausea, vomiting, diarrhea, rashes, fast breathing, rapid heartbeat, low blood pressure, seizures, headache, confusion, delirium, hallucinations, tremor and loss of coordination. The acute inflammatory condition may be associated with the presence of a cytokine storm and, for example, the subject may have one or more symptoms selected from high fever, swelling and redness, extreme fatigue, nausea, bleeding, clotting, internal organ injury and shock or any combination thereof.

The acute inflammatory condition may, in some embodiments, be associated with a multisystem inflammatory syndrome in children (MIS-C) wherein the subject, for example, has one or more symptoms that is new, selected from fever, vomiting, diarrhea, stomach pain, skin rash, red eyes, redness or swelling of the lips and tongue, feeling unusually tired, redness or swelling of the hands or feet, severe stomach pain, cardiac symptoms, including chest pain, palpitations and shortness of breath, bluish lips or face, mental confusion, inability to wake up or stay awake, abdominal pain with vomiting and diarrhea, skin rash and swelling of extremities, faintness and low blood pressure.

The acute inflammatory condition may also be associated with a systemic inflammatory response syndrome (SIRS). The subject may have one or more symptoms associated with a particular stage of SIRS as detailed below.

Stage 1 is a local reaction at the site of injury that aims at containing the injury and limit spread. Immune effector cells at the site release cytokines that in turn stimulate the reticuloendothelial system promoting wound repair through local inflammation. There is local vasodilatation induced by nitric oxide and prostacyclin and disruption of the endothelial tight junction to allow margination and transfer of leukocytes into tissue space. The leakage of cells and protein-rich fluid in extravascular space causes swelling and increased heat. Inflammatory mediators impact the local somatosensory nerves causing pain and loss of function. That loss of function also allows the part of the body to repair instead of persistent use.

Stage 2 is an early compensatory anti-inflammatory response syndrome in an attempt to maintain immunological balance. There is a stimulation of growth factors and recruitment of macrophages and platelets as the level of pro-inflammatory mediators decreases to maintain homeostasis.

Stage 3 is when the scale tips over towards pro-inflammatory SIRS resulting in progressive endothelial dysfunction, coagulopathy and activation of the coagulation pathway. It results in end-organ micro thrombosis, and a progressive increase in capillary permeability, eventually resulting in loss of circulatory integrity.

Stage 4 is characterised by compensatory anti-inflammatory response syndrome taking over SIRS, resulting in a state of relative immunosuppression. The individual, therefore, becomes susceptible to secondary or nosocomial infections, thus perpetuating the sepsis cascade.

Stage 5 manifests in multiple organ dysfunction syndrome with persistent dysregulation of both SIRS and compensatory anti-inflammatory response syndrome response.

In other embodiments, the acute inflammatory condition is associated with ARDS. In exemplary embodiments, the subject may have one or more symptoms selected from mild, moderate or severe hypoxemia as determined by Partial Pressure of arterial oxygen/Fraction of inspired oxygen (PaO₂/FiO₂) or positive end-expiratory pressure, bilateral opacities, respiratory failure, shortness of breath, laboured breathing, cough, fever, increased heart rate, low blood pressure, confusion, extreme tiredness, rapid breathing, organ failure, chest pain, bluish coloring of nails or lips, an change in the level of one or more inflammatory markers or need for mechanical ventilation.

In some embodiments, the acute inflammatory condition is associated with SARS wherein, for example, the subject may have one or more symptoms selected from acute febrile illness, malaise, fatigue, headache, flushing, diarrhea, nausea, vomiting, coughing including dry coughing, sore throat, runny nose, nasal congestion, production of pro-inflammatory mediators, vascular leakage and organ failure.

Also provided herein are methods of treating CRS or a cytokine storm in a subject, wherein the CRS or cytokine storm is associated with a coronavirus infection, comprising, consisting or consisting essentially of administering a compound of the invention to the subject. Also encompassed herein is a compound of the invention for use in treating CRS or a cytokine storm in a subject, wherein the CRS or cytokine storm is associated with a coronavirus infection, a use of a compound of the invention for treating CRS or a cytokine storm in a subject, wherein the CRS or cytokine storm is associated with a coronavirus infection, and a use of a compound of the invention in the manufacture of a medicament for treating CRS or a cytokine storm in a subject, wherein the CRS or cytokine storm is associated with a coronavirus infection.

In particular embodiments, the coronavirus is a betacoronavirus, such as a lineage A betacoronavirus, a lineage B betacoronavirus, a lineage C betacoronavirus or a lineage D betacoronavirus. In some embodiments, the betacoronavirus is a lineage B betacoronavirus, such as SARS-CoV or SARS-CoV-2; especially SARS-CoV-2. In alternative embodiments, the betacoronavirus is a lineage C betacoronavirus, such as MERS-CoV. In specific embodiments, the coronavirus is SARS-CoV-2.

Suitable embodiments of the compounds are discussed supra.

In particular embodiments, the CRS or cytokine storm comprises an elevation of at least 50% compared to basal state of one or more cytokines selected from IFN-γ, IFN-β, TNF-α, IL-1β, IL-6, IL-17A, CCL3 and CXCL2.

In some embodiments, the subject has CRS and has one or more symptoms selected from fever, fatigue, loss of appetite, muscle and joint pain, nausea, vomiting, diarrhoea, rashes, fast breathing, rapid heartbeat, low blood pressure, seizures, headache, confusion, delirium, hallucinations, tremor and loss of coordination.

In some embodiments, the subject has a cytokine storm and has one or more symptoms selected from high fever, swelling and redness, extreme fatigue, nausea, bleeding, clotting, internal organ injury and shock, or any combination thereof.

The CRS or cytokine storm may be associated with a MIS-C. In such embodiments, the subject may have one or more new symptoms selected from fever, vomiting, diarrhoea, stomach pain, skin rash, red eyes, redness or swelling of the lips and tongue, feeling unusually tired, redness or swelling of the hands or feet, severe stomach pain, cardiac symptoms, including chest pain, palpitations and shortness of breath, bluish lips or face, mental confusion, inability to wake up or stay awake, abdominal pain with vomiting and diarrhoea, skin rash and swelling of extremities, faintness and low blood pressure.

In some embodiments, the CRS or cytokine storm is associated with a SIRS, such as Stage 1 SIRS, Stage 2 SIRS, Stage 3 SIRS, Stage 4 SIRS or Stage 5 SIRS.

In particular embodiments, the CRS or cytokine storm is associated with ARDS. In these embodiments, the subject may have one or more symptoms selected from mild, moderate or severe hypoxemia as determined by Partial Pressure of arterial oxygen/Fraction of inspired oxygen (PaO₂/FiO₂) or positive end-expiratory pressure, bilateral opacities, respiratory failure, shortness of breath, laboured breathing, cough, fever, increased heart rate, low blood pressure, confusion, extreme tiredness, rapid breathing, organ failure, chest pain, bluish colouring of nails or lips, an change in the level of one or more inflammatory markers, or need for mechanical ventilation.

In particular embodiments, the CRS or cytokine storm is associated with SARS. For example, the subject may have one or more symptoms selected from acute febrile illness, malaise, fatigue, headache, flushing, diarrhoea, nausea, vomiting, coughing including dry coughing, sore throat, runny nose, nasal congestion, production of pro-inflammatory mediators, vascular leakage and organ failure.

Also provided herein are methods of treating SARS in a subject, wherein the SARS is associated with a coronavirus infection, comprising, consisting or consisting essentially of administering a compound of the invention to the subject. Further aspects include a compound of the invention for use in treating SARS in a subject, wherein the SARS is associated with a coronavirus infection, a use of a compound of the invention for treating SARS in a subject, wherein the SARS is associated with a coronavirus infection, and a use of a compound of the invention in the manufacture of a medicament for treating SARS in a subject, wherein the SARS is associated with a coronavirus infection.

In particular embodiments, the coronavirus is a betacoronavirus, such as a lineage A betacoronavirus, a lineage B betacoronavirus, a lineage C betacoronavirus or a lineage D betacoronavirus. In some embodiments, the betacoronavirus is a lineage B betacoronavirus, such as SARS-CoV or SARS-CoV-2; especially SARS-CoV-2. In alternative embodiments, the betacoronavirus is a lineage C betacoronavirus, such as MERS-CoV. In specific embodiments, the coronavirus is SARS-CoV-2.

Suitable embodiments of the compounds are discussed supra.

In particular embodiments, the subject has one or more symptoms selected from acute febrile illness, malaise, fatigue, headache, flushing, diarrhoea, nausea, vomiting, coughing including dry coughing, sore throat, runny nose, nasal congestion, production of pro-inflammatory mediators, vascular leakage and organ failure, or any combination thereof.

In some embodiments, the SARS is associated with presence of CRS or a cytokine storm, for example, wherein the CRS or cytokine storm comprises an elevation of at least 50% compared to basal state of one or more cytokines selected from IFN-γ, IFN-β, TNF-α, IL-1β, IL-6, IL-17A, CCL3 and CXCL2.

The SARS may also be associated with a MISC-C wherein, for example, the subject may have one or more new symptoms selected from fever, vomiting, diarrhoea, stomach pain, skin rash, red eyes, redness or swelling of the lips and tongue, feeling unusually tired, redness or swelling of the hands or feet, severe stomach pain, cardiac symptoms, including chest pain, palpitations and shortness of breath, bluish lips or face, mental confusion, inability to wake up or stay awake, abdominal pain with vomiting and diarrhoea, skin rash and swelling of extremities, faintness and low blood pressure.

The SARS may also be associated with a SIRS. For example, the subject may have Stage 1 SIRS, Stage 2 SIRS, Stage 3 SIRS, Stage 4 SIRS or Stage 5 SIRS as discussed supra.

Suitable coronaviruses and embodiments of the compounds of the invention are as discussed supra.

Also contemplated herein is the use of the compounds of the invention for inhibiting damage or impairment of a cell infected with a coronavirus. Further provided herein is a method of inhibiting damage or impairment of a cell infected with a coronavirus, comprising, consisting or consisting essentially of contacting the cell with a compound of the invention, a compound of the invention for use in inhibiting damage or impairment of a cell infected with a coronavirus, a use of a compound of the invention for inhibiting damage or impairment of a cell infected with a coronavirus, and a use of a compound of the invention for inhibiting the damage or impairment of a cell infected with a coronavirus.

The damage or impairment may be any damage or impairment associated with NSP5 activity, and may, for example, lead to one or more neurological symptoms associated with a coronavirus infection, such as fatigue, anosmia, dysgeusia, loss of taste, muscle ache, headache, dizziness, seizure, stroke, muscular weakness, impaired concentration, loss of memory, delirium, pain, depression, psychosis, brain inflammation, Guillain-Barré syndrome and the like.

In particular embodiments, the cell is host cell (e.g. a human cell), especially a neuron.

Suitable coronaviruses and embodiments of the compounds of the invention are as discussed supra.

Also provided are methods of treating or inhibiting the development of a condition in a subject in which inhibiting NSP5 activity is associated with effective treatment or inhibition, comprising, consisting or consisting essentially of administering a compound of the invention to the subject. Also provided is a compound of the invention for use in treating or inhibiting the development of a condition in a subject in which inhibiting NSP5 activity is associated with effective treatment or inhibition, a use of a compound of the invention for treating or inhibiting the development of a condition in a subject in which inhibiting NSP5 activity is associated with effective treatment or inhibition, and a use of a compound of the invention in the manufacture of a medicament for treating or inhibiting the development of a condition in a subject in which inhibiting NSP5 activity is associated with effective treatment or inhibition.

In particular embodiments, NSP5 is coronavirus NSP5.

Suitable conditions include, but are not limited to, a viral infection, such as a coronavirus infection; a respiratory condition associated with a coronavirus infection, such as an acute inflammatory condition, ARDS, CRS or a cytokine storm or SARS; or a neurological condition and/or symptom associated with a coronavirus infection.

While administration to any subject is contemplated, in any one of the above aspects, the subject has or is suspected of having a coronavirus infection, or has been exposed to or is at risk of being exposed to a coronavirus capable of causing a viral infection. In particular embodiments, the subject is a human.

Any one of the aspects described above may involve administration of an effective amount of the compound of the invention as described supra. The compound may be administered in the form of, for example, a composition as described herein. The compound of the invention may be administered to the subject by any route of administration suitable to effect treatment or inhibition of the development of the coronavirus infection or treatment or inhibition of the development of the condition. A skilled person will be well aware of suitable routes of administration, such as one or more of the routes of administration discussed supra. For example, in some embodiments, the compound is administered by oral, inhalation, intranasal, topical or intravenous (e.g. infusion) administration; especially oral, inhalation or intranasal administration.

The dosage and frequency of administration will depend on the subject, the disorder to be treated and the route of administration. A skilled person will readily be able to determine suitable dosages and frequency of such dosages. For example, the compound may be administered in an amount in the range of from about 0.25 μg to about 2000 mg, and may be administered at a frequency of, for example, once daily, or twice or three times daily. The treatment may be continued for multiple days, weeks, months or years. In embodiments where the pharmaceutical composition comprises one or more additional active agents, the dosages and frequency of administration are determined by reference to the usual dose and manner of administration of the said agents.

Any one of the methods described above may, in some embodiments, involve the administration of one or more further active agents as described supra, such as an antiviral agent.

A skilled person would be well aware of suitable assays used to evaluate inhibition of NSP5. For example, the method may include contacting NSP5 (e.g. immobilised NSP5) with a compound and assessing the binding affinity or the inhibition of the enzymatic activity, e.g. proteolytic activity. Alternatively, the method may include screening for the inhibition of the activity, presence or expression of a downstream cellular target or product, or a downstream effect, such as assembly of the viral replication transcription complex, the presence of a viral infection in a subject, such as an animal model, or the presence of a cleaved substrate, e.g. one or more of non-structural protein 4 (NSP4) to non-structural protein 16 (NSP16). Detecting such inhibition may be achieved utilising techniques including, but not limited to, ELISA, a binding assay (e.g. a radioligand binding assay or fluorescence binding assay), surface plasmon resonance, immunofluorescence, Western blots, immunoprecipitation, immunostaining, scintillation proximity assays, competitive inhibition assays, a colorimetric assay, cell proliferation assays and neuronal cell viability assays as described further in the examples herein. Commercially available kits and/or products may also be used, such as 3CL Protease (SARS-CoV-1) Assay Kit (Catalogue No. 78015; BPS Bioscience Inc., San Diego, USA), the SARS-CoV-2 Main Protease Inhibitor Screening Assay Kit (Catalogue No. 701960; Cayman Chemical, Ann Arbor, Michigan, USA) or the SensoLyte® SARS-CoV-2 3CL Protease Activity Assay Kit (Catalogue No. AS-72262; AnaSpec, Fremont, California, USA). Animal models may also be used to assess viral infection.

In order that the invention may be readily understood and put into practical effect, particular preferred embodiments will now be described by way of the following non-limiting examples.

### EXAMPLES

All chemicals, starting materials, antibodies and reagents are commercially available from, for example, Sigma-Aldrich (St Louis, USA), Thermo Fisher Scientific (Waltham, USA), Abcam (Cambridge, United Kingdom) and the like, unless otherwise stated.

### Example 1—Identifying NSP5 Inhibitors Using Virtual Screening

### Materials and Methods

The virtual screening was carried out in Schrodinger Software package. The small molecule database was prepared from Enamine Discovery Diversity Set (Enamine, Kyiv, Ukraine) and consisted of 60,480 compounds. The 2D structures were converted into 3D structure using the Ligprep function within Schrödinger package (Schrödinger, Inc., New York, USA). The crystal structure of NSP5 was prepared by downloading the pdb file from Protein Data Bank (PDB: 6Y2G). The protein was prepared using protein preparation wizard within the Schrödinger package using default settings. Receptors grids were set up as 10 Å radius around the protease active pocket between Cys 145 and His 41. Small molecules were docked into the receptor using the Glide SP method, and the pose with the lowest GLIDE binding score was selected for study. Schrödinger Maestro 12.9 (Schrödinger, Inc., New York, USA) was then used to visualise the interactions between the ligand molecules.

### Results

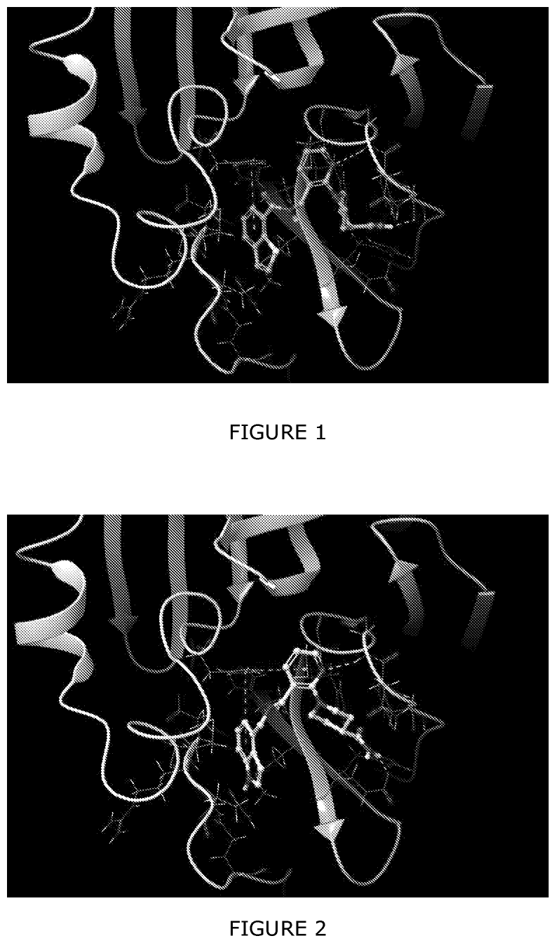

The virtual screening identified compounds that bind to the active protease site of NSP5 with comparable binding scores, and which were subsequently shown to inhibit SARS-CoV-2 NSP5 activity (discussed below). These compounds, referred to herein as compound 2 [C2; Formula (IV)] and compound 22 [C22; Formula (V)], and their docking scores are provided in Table 1. In silico docking shows compound 2 docked into the protease active centre of NSP5 (FIG. 1) and compound 22 docked into the same position (FIG. 2).

TABLE 1

| Compound | | |
|---|---|---|
| No. | Structure | Docking Score |
| 2 | 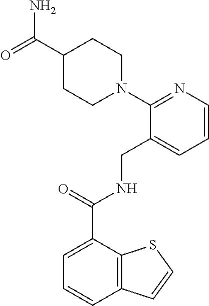 [SMILES: NC(=O)C1CCN(c2ncccc2CNC(=O)c2cccc3ccsc23)CC1] | -8.081 |
| 22 | 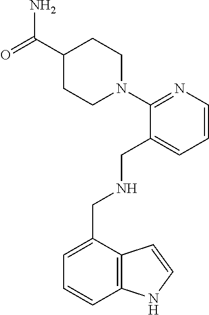 [SMILES: NC(=O)C1CCN(c2ncccc2CNCc2cccc3[nH]ccc23)CC1] | -7.629 |

### Example 2—Characterisation of Compounds 2 and 22

Compounds 2 and 22 were synthesised by Enamine Ltd (Kyiv, Ukraine).

Characterisation data for compound 2: ¹H NMR (DMSO-d₆, 500 MHz): 9.28 (t, J=5.6 Hz, 1H, NH), 8.18 (dd, J=4.7, 1.8 Hz, 1H, ArH), 8.80 (dd, J=15.2, 7.9 Hz, 2H, ArH), 7.83 (d, J=5.6 Hz, 1H, ArH), 7.60 (dd, J=7.5, 1.6 Hz, 1H, ArH), 7.54-7.49 (m, 2H, ArH), 7.31 (s, 1H, NH), 7.00 (dd, J=7.5, 4.9 Hz, 1H, ArH),6.78 (s, 1H, NH), 4.57 (d, J=5.6 Hz, 2H, CH₂), 3.41 (d, J=12.9 Hz, 2H, CH₂), 2.79 (m, 2H, CH₂), 2.29 (m, 1H, CH), 1.88-1.72 (m, 4H, 2×CH₂); ¹³C{¹H}NMR (DMSO-d₆, 125 MHz): 176.9, 166.6, 161.3, 146.2, 141.3, 138.7, 136.4, 131.4, 127.9, 127.3, 126.7, 124.5, 123.4, 123.2, 118.6, 50.6, 42.2, 38.7, 29.2; ESI-MS: [M+H]⁺ found 395.05, calculated 395.15.

Characterisation data for compound 22: ¹H NMR (DMSO-d₆, 500 MHz): 11.05 (s, 1H, NH), 8.13 (m, 1H, ArH), 7.83 (dd, J=7.6, 1.9 Hz, 1H, ArH), 7.32-7.22 (m, 3H, ArH), 7.07-6.94 (m, 3H, ArH), 6.74 (s, 1H, NH), 6.50 (m, 1H, NH), 3.95 (s, 2H, CH₂), 3.70 (s, 2H, CH₂), 3.45 (m, 2H, CH₂), 2.67 (m, 2H, CH₂), 2.20 (m, 1H, NH), 1.71 (m, 2H, CH₂), 1.62 (m, 2H, CH₂); ¹³C{¹H}NMR (DMSO-d₆, 125 MHz): 177.0, 161.8, 145.9, 138.1, 136.3, 132.2, 128.0, 127.3, 125.1, 121.2, 118.4, 118.2, 110.5, 99.9, 51.6, 50.5, 48.7, 42.2, 29.2; ESI-MS: [M+H]⁺ found 364.15, calculated 364.21.

### Example 3—Effect of Compounds 2 and 22 on Neurons in the Presence of NSP5

### Materials and Methods

Primary neurons obtained from mouse embryos and cultured in Neurobasal medium supplemented with B27 and glutamax were treated with 5×10¹⁰ titers of adeno-associated virus (AAV) 9-GFP (AAV9-GFP), AAV9-NSP5-V5 or AAV9-NSP5 C145A-V5 (the enzyme deficient mutant) at DIV 11 (days in vitro). In AAV9-GFP constructs GFP expression is driven by a neuronal Syn1 promoter. AAV9-NSP5-V5 confers the expression of the SARS-CoV-2 NSP5 together with a C-terminal V5 protein tag for detection purposes driven by the Syn1 promoter. AAV9-NSP5 C145A-V5 expresses a protease-inactive variant of the SARS-CoV-2 NSP5 in neurons. AAV were produced by plasmid transfection of human embryonic kidney 293 cells and extracted from cell lysates. 0.4, 2, 10 or 50 μM of compound 2 or compound 22 (prepared according to Example 2) was added to AAV9-NSP5-V5 treated neurons two days after AAV treatment. Cells were then fixed with 4% paraformaldehyde (PFA) four days after AAV treatment or two days following addition of the test compounds. Fixed cells were stained with rabbit anti-neuronal βIII-tubulin, mouse anti-V5, and DAPI. Following that, fixed cells were treated with secondary antibodies of anti-rabbit 555 (red) and anti-mouse 488 (green). Images were taken using Olympus BX51 fluorescence microscope.

### Results

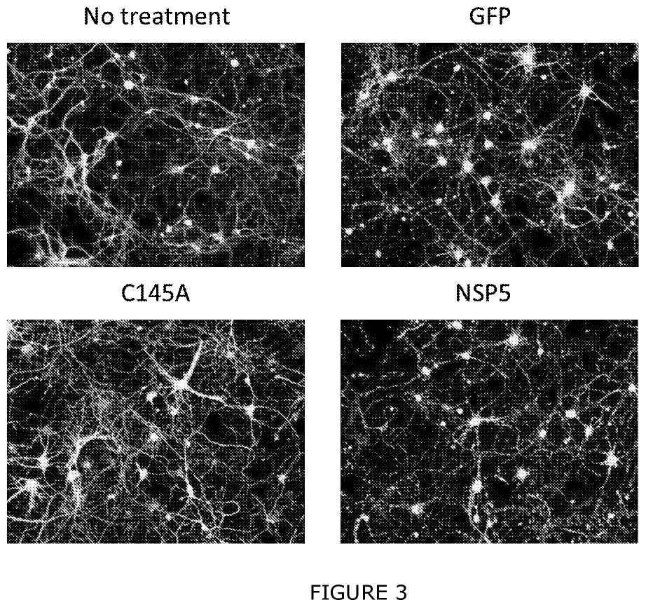

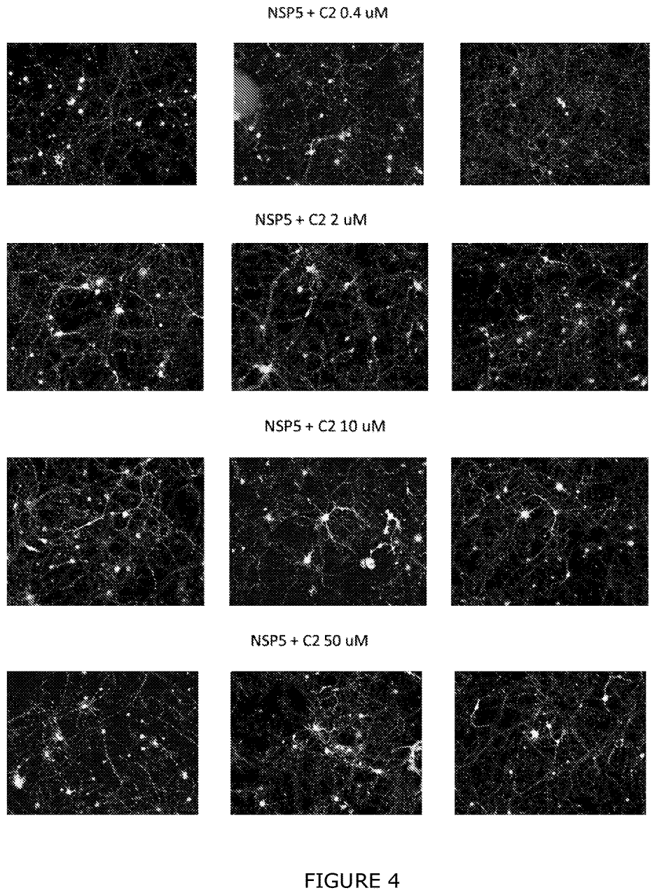

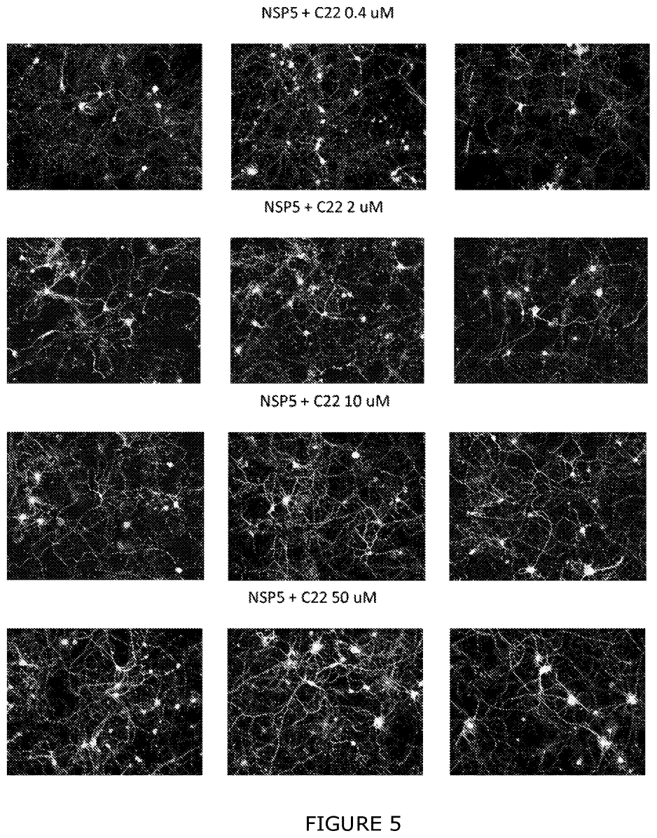

Results are presented in FIGS. 3-5. Compounds 2 (FIG. 4) and 22 (FIG. 5) prevented NSP5-induced neuronal death in a dose-dependent manner. FIG. 3 shows the toxic effects of expression of NSP5 in primary neurons (staining for structural tubulin (red) and GFP or NSP5 (green)). Expression of the protease-dead C145A variant of NSP5 showed no toxicity. This suggests that the protease activity of NSP5 confers neurotoxicity. Hence, inhibition of NSP5 protease activity is beneficial to neuronal survival. FIG. 4 shows the protective effects of compound 2 (C2) at concentrations of 0.4, 2, 10 and 50 μM against NSP5-induced neurotoxicity. FIG. 5 shows the protective effects of compound 22 (C22) at concentrations of 0.4, 2, 10 and 50 μM against NSP5-induced neurotoxicity.

### Example 4—Cytotoxicity of Compounds 2 and 22

### Materials and Methods

Primary neurons were treated with 0.4, 1, 2, 10 or 50 μM of compound 2 or compound 22 (prepared according to Example 2). NSP5 was added to primary neurons at DIV11, and compound 2 or 22 was added 48 hours later. Cells were treated with propidium iodide 48 hours after compound addition. Cells were washed with PBS and fixed with 4% PFA. Cells were then washed and stained with DAPI. Images of cells were taken with ZEISS Axioscan 7 microscope slide scanner. Cell death was determined by Image J analysis, counting the death cell (red, PI)) vs all cells (blue, DAPI).

One hour after compound addition (1 or 10 μM of compound 2 or compound 22) neurons were treated with a cytotoxic agent (100 μM N-methyl-D-aspartate (NMDA), 100 μM H₂O₂ or 5 μM staurosporine). One hour after treatment with the cytotoxic agent, the media was replaced with fresh media and the neurons were incubated for 24 hours. A final concentration of 1 μM propidium iodide (PI) was added into the well and incubated for 5 mins. Cells were washed with phosphate-buffered saline (PBS) and then fixed with 4% PFA. Cells were then washed and stained with DAPI, and cell death determined as described above.

### Results

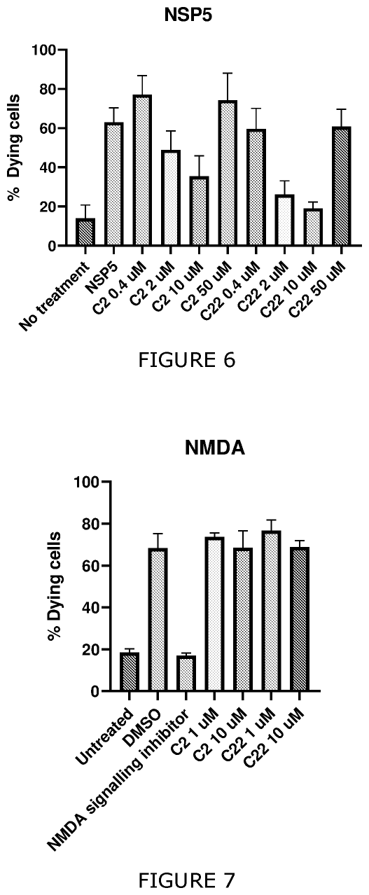

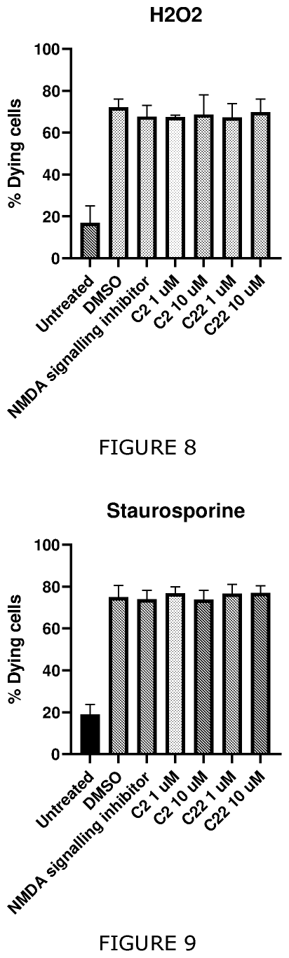

The results are presented in FIGS. 6-9. As shown in FIG. 6, compounds 2 and 22 did not cause any cell death at therapeutic concentrations (nM to low μM) and selectively reduced neuronal cell death caused by NSP5 (FIG. 6). Compound 2 and 22 did not protect from NMDA-induced neurotoxicity (FIG. 7), hydrogen peroxide (H₂O₂)-induced neurotoxicity (FIG. 8) or staurosporine-induced neurotoxicity (FIG. 9).

### Example 5—Design and Synthesis of Analogues of Compounds 2 and 22

Analogues of compounds 2 and 22 were designed and are presented in Table 2. Compounds 32-40 were synthesised by Enamine Ltd (Kyiv, Ukraine).

TABLE 2

| Compound | |
|---|---|
| No. | Structure |
| 31 | 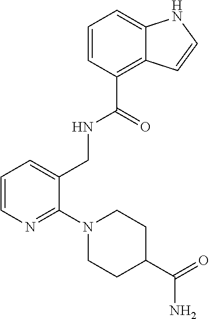 [SMILES: NC(=O)C1CCN(c2ncccc2CNC(=O)c2cccc3[nH]ccc23)CC1] |
| 32 | 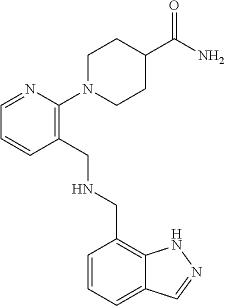 [SMILES: NC(=O)C1CCN(c2ncccc2CNCc2cccc3cn[nH]c23)CC1] |
| 33 | 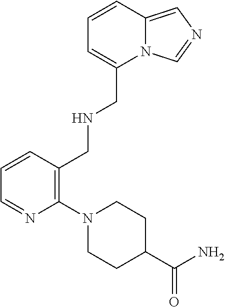 [SMILES: NC(=O)C1CCN(c2ncccc2CNCc2cccc3cncn23)CC1] |
| 34 | 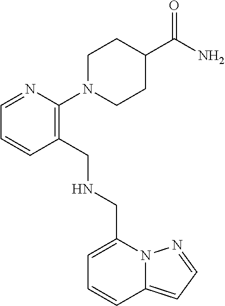 [SMILES: NC(=O)C1CCN(c2ncccc2CNCc2cccc3ccnn23)CC1] |
| 35 | 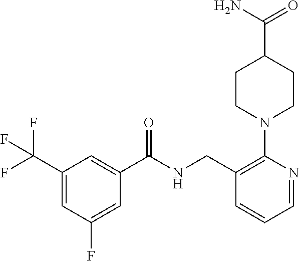 [SMILES: NC(=O)C1CCN(c2ncccc2CNC(=O)c2cc(F)cc(C(F)(F)F)c2)CC1] |
| 36 | 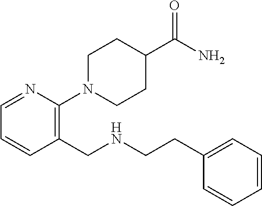 [SMILES: NC(=O)C1CCN(c2ncccc2CNCCc2ccccc2)CC1] |
| 37 | 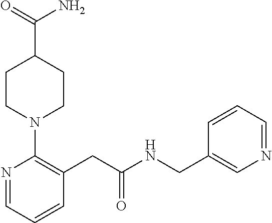 [SMILES: NC(=O)C1CCN(c2ncccc2CC(=O)NCc2cccnc2)CC1] |
| 38 | 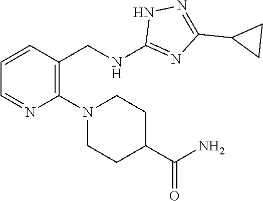 [SMILES: NC(=O)C1CCN(c2ncccc2CNc2nc(C3CC3)n[nH]2)CC1] |
| 39 | 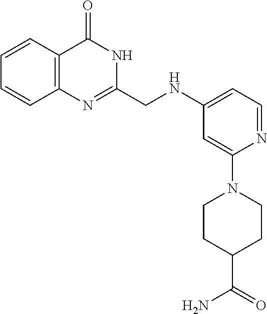 [SMILES: NC(=O)C1CCN(c2cc(NCc3nc4ccccc4c(=O)[nH]3)ccn2)CC1] |
| 40 | 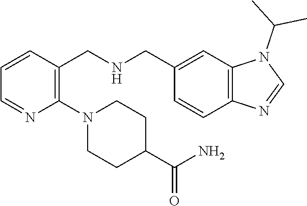 [SMILES: CC(C)n1cnc2ccc(CNCc3cccnc3N3CCC(C(N)=O)CC3)cc21] |

An outline of the synthetic procedure for compound 31 is provided below:

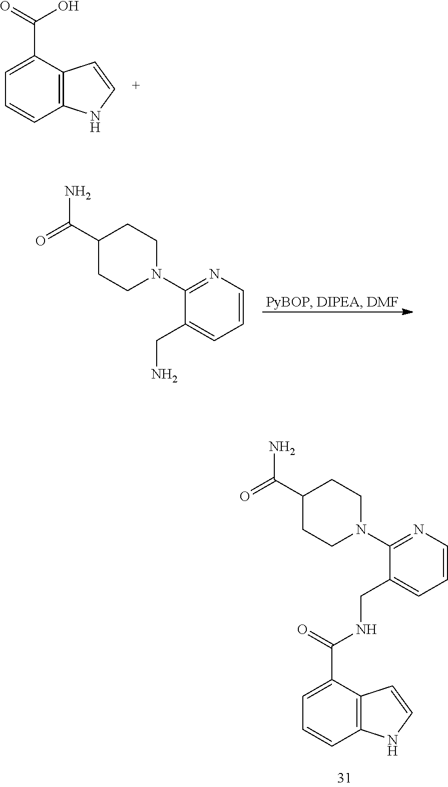 [SMILES: O=C(O)c1cccc2[nH]ccc12 ; NCc1cccnc1N1CCC(C(N)=O)CC1 ; NC(=O)C1CCN(c2ncccc2CNC(=O)c2cccc3[nH]ccc23)CC1]

In brief, 1-(3-(aminomethyl)pyridin-2-yl)piperidine-4-carboxamide (100 mg, 0.43 mmol) and 1H-indole-4-carboxylic acid (137 mg, 0.85 mmol) were dissolved in dimethylformamide (DMF; 7 mL). Benzotriazole-1-yl-oxy-tris-pyrrolidino-phosphonium hexafluorophosphate (PyBOP; 671 mg, 1.29 mmol) and N,N-diisopropylethylamine (DIPEA; 0.45 mL) were added to the reaction mixture and the resulting solution was stirred overnight at room temperature. The reaction mixture was diluted with 50% acetonitrile in water and then subjected to high-performance liquid chromatography (HPLC) purification. The total yield was 35 mg (22%).

An outline of the synthetic procedure for compound 32 is provided below:

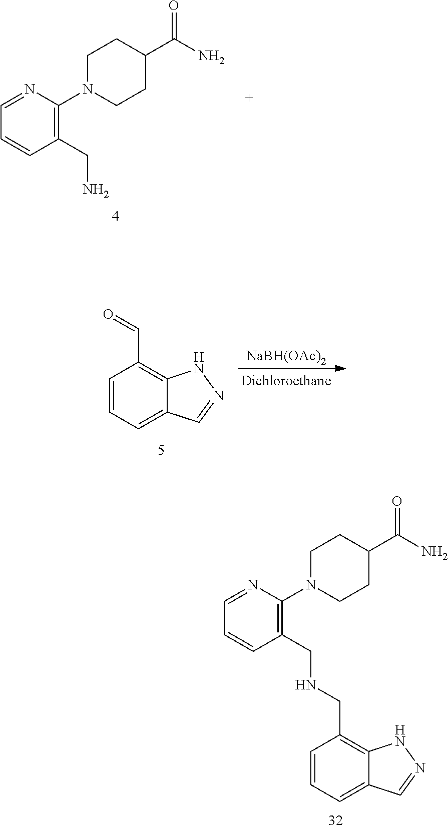 [SMILES: NCc1cccnc1N1CCC(C(N)=O)CC1 ; O=Cc1cccc2cn[nH]c12 ; NC(=O)C1CCN(c2ncccc2CNCc2cccc3cn[nH]c23)CC1 ; "NaBH(OAc)2"]

In brief, to a stirring solution of 4 and 5 in dichloroethane, NaBH(OAc)₃ was added to undergo reductive amination to obtain compound 32.

An outline of the synthetic procedure for compound 33 is provided below:

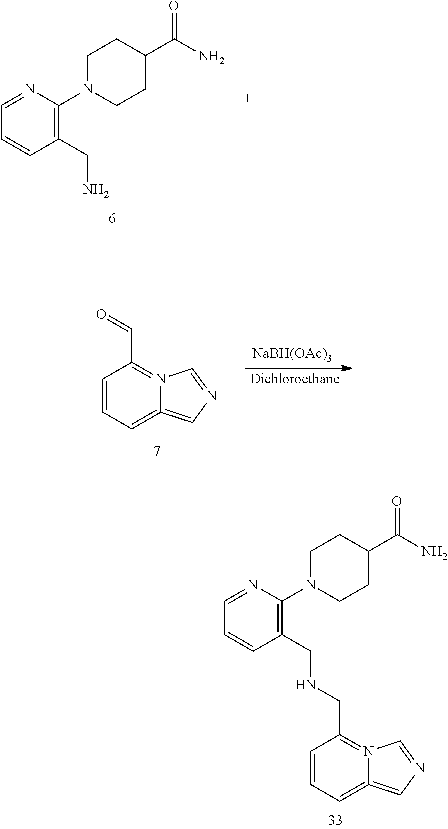

In brief, to a stirring solution of 6 and 7 in dichloroethane, NaBH(OAc)₃ was added to undergo reductive amination to obtain compound 33.

An outline of the synthetic procedure for compound 34 is provided below:

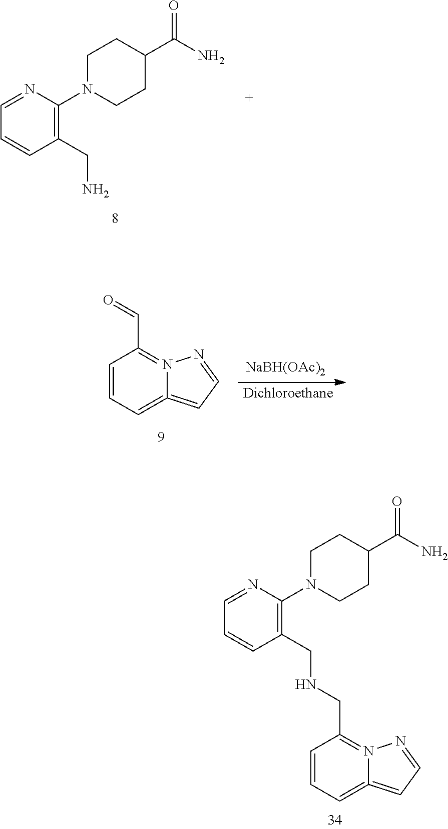

In brief, to a stirring solution of 8 and 9 in dichloroethane, NaBH(OAc)₃ was added to undergo reductive amination to obtain compound 34.

An outline of the synthetic procedure for compound 35 is provided below:

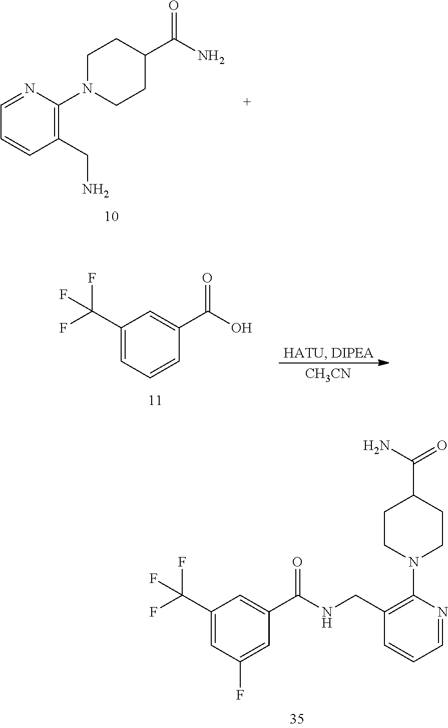 [SMILES: NCc1cccnc1N1CCC(C(N)=O)CC1 ; O=C(O)c1cccc(C(F)(F)F)c1 ; "CH3CN" ; "HATU, DIPEA" ; NC(=O)C1CCN(c2ncccc2CNC(=O)c2cc(F)cc(C(F)(F)F)c2)CC1]

In brief, an amide bond was formed by reaction of 10 and 11 in the presence of 1-[bis(dimethylamino)methylene]-1H-1,2,3-triazolo[4,5-b]pyridinium 3-oxid hexafluorophosphate (HATU) and DIPEA in acetonitrile (CH₃CN) to obtain compound 35.

An outline of the synthetic procedure for compound 36 is provided below:

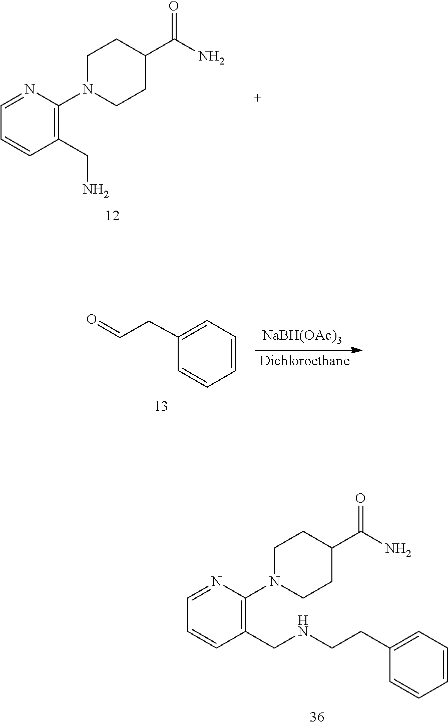

In brief, to a stirring solution of 12 and 13 in dichloroethane, NaBH(OAc)₃ was added to undergo reductive amination to obtain compound 36.

An outline of the synthetic procedure for compound 37 is provided below:

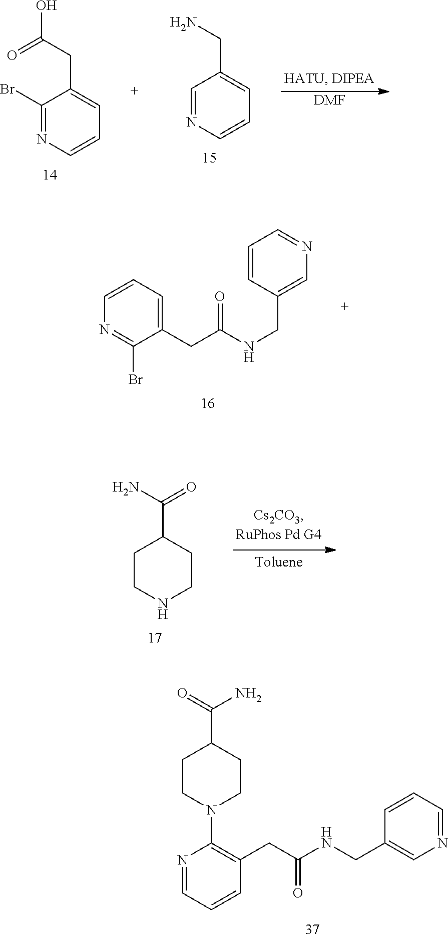

In brief, precursor 16 was formed by amidation reaction of 14 and 15 in the presence of HATU and DIPEA in DMF. Precursor 16 was then reacted with piperidine 17 in the presence of a RuPhos Pd G4 ([dicyclohexyl(2′,6′-diisopropoxy-2-biphenylyl)phosphine-κP](methanesulfonatato-κO)[2′-(methylamino-κN)-2-biphenylyl-κC²]palladium) catalyst to form compound 37.

An outline of the synthetic procedure for compound 38 is provided below:

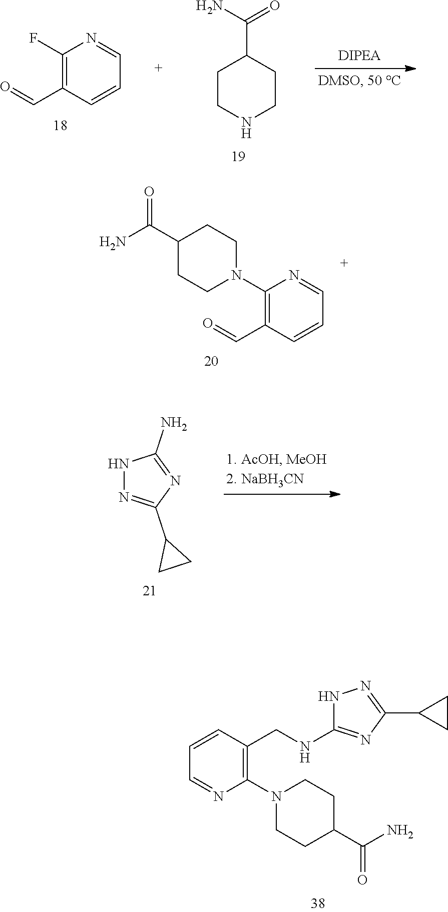 [SMILES: O=Cc1cccnc1F ; NC(=O)C1CCNCC1 ; NC(=O)C1CCN(c2ncccc2C=O)CC1 ; Nc1nc(C2CC2)n[nH]1 ; NC(=O)C1CCN(c2ncccc2CNc2nc(C3CC3)n[nH]2)CC1]

Nucleophilic aromatic substitution of 18 and 19 took place in the presence of DIPEA in dimethyl sulfoxide (DMSO) at 50 °C followed by reductive amination with triazole 21 in acetic acid (AcOH) and methanol (MeOH) in the presence of sodium cyanoborohydride (NaBH₃CN) to give compound 38.

An outline of the synthetic procedure for compound 39 is provided below:

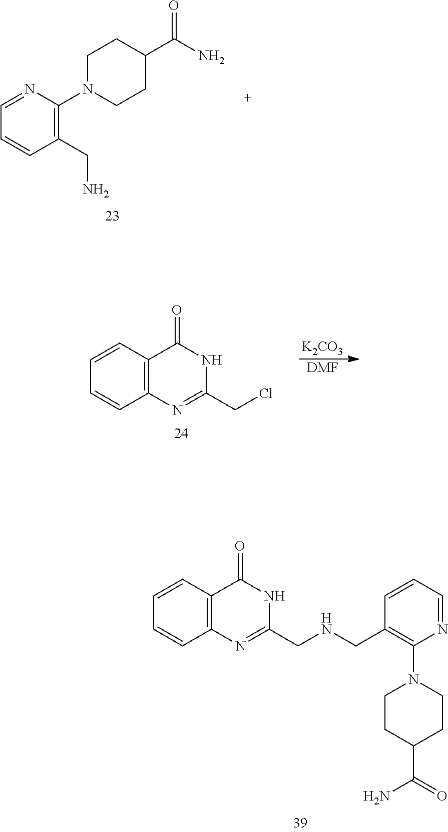

Amination of 23 and 24 with K₂CO₃ in DMF gave compound 39.

An outline of the synthetic procedure for compound 40 is provided below:

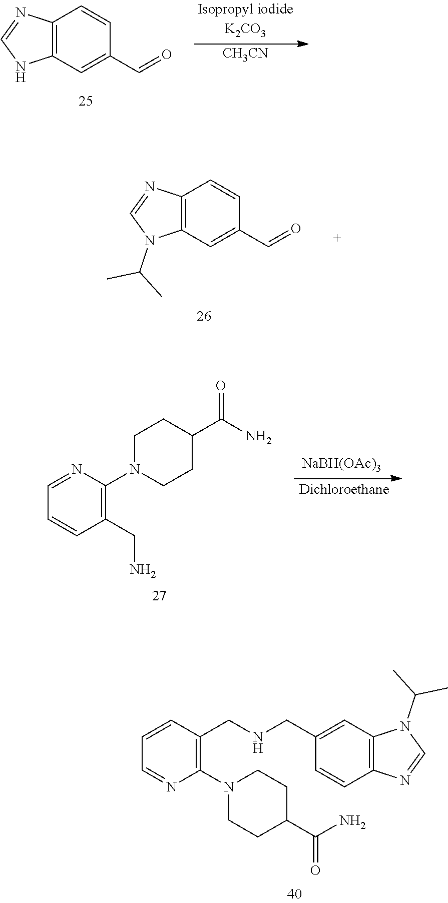

25 was alkylated with isopropyl iodide to give precursor 26. Reductive amination of precursor 26 with 27 in the presence of sodium triacetoxyborohydride (NaBH (OAc)₃ formed compound 40.

The compounds were analysed using NMR spectroscopy and mass spectrometry (MS). The NMR and MS results are as follows:

Compound 31: ¹H NMR (DMSO-d₆, 400 MHz): 11.25 (s, 1H, NH), 8.76 (t, J=5.1 Hz, 1H, NH), 7.71 (d, J=6.6 Hz, 1H, ArH), 7.49 (d, J=8.5 Hz, 1H, ArH), 7.43 (d, J=7.1 Hz, 1H, ArH), 7.37 (m, 1H, ArH), 7.26 (s, 1H, ArH), 7.07 (m, 2H, ArH), 6.77 (m, 2H, ArH), 4.45 (d, J=5.7 Hz, 2H, CH₂), 3.43 (d, J=10.6 Hz, 1H, CH), 2.84 (d, J=10.6 Hz, 2H, CH₂), 2.26 (m, 1H, CH), 1.84-1.64 (m, 4H, 2×CH₂), MS (ESI+): [C₂₁H₂₃N₅O₂+H]⁺ calc. 378.19, found 378.45.

Compound 32: ¹H NMR (DMSO-d₆, 500 MHz): 13.05 (s, 1H, NH), 8.12 (m, 2H, ArH), 7.83 (d, J=5.0 Hz, 1H, ArH), 7.62 (d, J=8.0 Hz, 1H, ArH), 7.25 (m, 2H, ArH), 7.02 (m, 2H, ArH), 6.73 (s, 1H, NH), 4.02 (s, 2H, CH₂), 3.66 (s, 2H, CH₂), 3.30 (m, 2H, CH₂), 2.65 (m, 3H, CH₂, CH), 2.14 (m, 1H, CH), 1.69-1.49 (m, 4H, 2×CH₂); MS (ESI+): [C₂₀H₂₄N₆O+H]⁺ calc. 365.20, found 365.45.

Compound 33: ¹H NMR (DMSO-d₆, 500 MHz): 8.45 (s, 1H, NH), 8.03 (dd, J=7.5, 1.9 Hz, 1H, ArH), 7.75 (dd, J=7.0, 1.8 Hz, 1H, ArH), 7.42 (m, 2H, ArH), 6.88 (m, 1H, ArH), 6.77 (m, 1H, ArH), 6.63 (m, 1H, ArH), 4.12 (s, 2H, CH₂), 3.56 (s, 2H, CH₂), 3.32 (m, 3H, CH₂, CH), 2.29 (m, 1H, CH), 1.73-1.60 (m, 4H, 2×CH₂); MS (ESI+): [C₂₀H₂₄N₆O+H]⁺ calc. 365.20, found 365.40.

Compound 34: ¹H NMR (DMSO-d₆, 500 MHz): 8.12 (m, 1H, ArH), 7.97 (t, J=5.1 Hz, 1H, ArH), 7.78 (m, 1H, ArH), 7.62 (m, 2H, ArH), 7.20 (m, 2H, ArH), 6.95 (m, 2H, ArH), 6.83 (s, 1H, NH), 6.62 (d, J=5.0 Hz, 1H, ArH), 4.12 (d, J=8.2 Hz, 2H, CH₂), 3.74 (d, J=8.1 Hz, 2H, CH₂), 3.33 (m, 1H, CH), 3.06 (m, 1H, CH), 2.63 (m, 2H, CH₂), 2.17 (m, 1H, CH), 1.82-1.55 (m, 4H, 2×CH₂); MS (ESI+): [C₂₀H₂₄N₆O+H]⁺ calc. 365.20, found 365.40.

Compound 35: ¹H NMR (DMSO-d₆, 500 MHz): 9.29 (t, J=7.0 Hz, 1H, NH), 8.17-7.92 (m, 4H, ArH), 7.57 (m, 1H, ArH), 7.26 (s, 1H, ArH), 6.99 (m, 1H, ArH), 6.84 (s, 1H, ArH), 4.49 (m, 2H, CH₂), 3.34 (m, 2H, CH₂), 2.75 (t, J=7.5 Hz, CH₂), 2.25 (m, 1H, CH), 1.82-1.66 (m, 4H, 2×CH₂); MS (ESI+): [C₂₀H₂₀F₄N₄O₂+H]⁺ calc. 425.15, found 425.30.

Compound 36: ¹H NMR (DMSO-d₆, 500 MHz): 8.09 (dd, J=7.9, 1.4 Hz, 1H, ArH), 7.67 (dd, J=7.2, 1.4 Hz, 1H, ArH), 7.27-7.10 (m, 6H, ArH), 6.93 (m, 1H, ArH), 6.73 (s, 1H, NH), 3.65 (s, 2H, CH₂), 3.40 (d, J=12 Hz, 2H, CH₂), 2.74-2.60 (m, 6H, 3×CH₂), 2.46-2.07 (m, 2H, CH₂), 1.74-1.58 (m, 4H, 2×CH₂); MS (ESI+): [C₂₀H₂₆N₄O+H]⁺ calc. 339.21, found 339.45.

Compound 37: ¹H NMR (DMSO-d₆, 400 MHz): 8.67 (t, J=5.1 Hz, 1H, NH), 8.47 (m, 2H, ArH), 8.16 (dd, J=4.8, 1.9 Hz, 1H, ArH), 7.67 (m, 1H, ArH), 7.55 (dd, J=7.4, 1.8 Hz, 1H, ArH), 7.35 (m, 1H, ArH), 7.28 (s, 1H, NH), 6.99 (m, 1H, ArH), 6.75 (s, 1H, NH), 4.32 (d, J=5.8 Hz, 1H, CH₂), 3.54 (s, 2H, CH₂), 2.67 (m, 2H, CH₂), 2.46 (m, 1H, CH), 2.21 (m, 1H, CH), 1.79-1.67 (m, 4H, 2×CH₂); MS (ESI+): [C₁₉H₂₃N₅O₂+H]⁺ calc. 354.19, found 354.40.

Compound 38: ¹H NMR (DMSO-d₆, 500 MHz): 12.42 (s, 1H, NH), 11.78 (s, 1H, NH), 8.12 (d, J=11.0 Hz, 1H, ArH), 7.57 (m, 1H, ArH), 7.26 (s, 1H, ArH), 6.95 (m, 1H, ArH), 6.82-6.07 (m, 2H, ArH), 4.22 (d, J=9.2 Hz, 2H, CH₂), 3.33 (m, 2H, CH₂), 2.72 (m, 2H, CH₂), 2.19 (m, 1H, CH), 1.85-1.61 (m, 5H, 2×CH₂, CH), 0.90-0.60 (m, 4H, 2×CH₂); MS (ESI+): [C₁₇H₂₃N₇O+H]⁺ calc. 342.20, found 342.45.

Compound 39: ¹H NMR (DMSO-d₆, 500 MHz): 8.09 (m, 2H, ArH), 7.77 (m, 2H, ArH), 7.63 (d, J=7.6 Hz, 1H, ArH), 7.47 (t, J=7.4 Hz, 1H, ArH), 7.45 (s, 1H, NH), 6.96 (m, 1H, ArH), 6.71 (s, 1H, ArH), 3.68 (d, J=9.0 Hz, 4H, 2×CH₂), 3.38 (d, J=7.8 Hz, 2H, CH₂), 2.65 (m, 2H, CH₂), 2.18 (m, 1H, CH), 1.70-1.55 (m, 4H, 2×CH₂); MS (ESI+): [C₂₁H₂₄N₆O₂+H]⁺ calc. 393.20, found 393.40.

Compound 40: ¹H NMR (DMSO-d₆, 500 MHz): 8.26 (s, 1H, ArH), 8.13 (m, 1H, ArH), 7.82 (d, J=5.2 Hz, 1H, ArH), 7.65 (m, 1H, ArH), 7.28-7.17 (m, 2H, ArH), 6.94 (m, 1H, ArH), 6.81 (s, 1H, NH), 4.69 (m, 1H, CH), 3.82 (s, 2H, CH₂), 3.65 (s, 2H, CH₂), 3.42 (d, J=8.0 Hz, 2H, CH₂), 2.65 (m, 3H, CH₂, CH), 2.16 (m, 1H, CH), 1.75-1.57 (m, 4H, 2×CH₂), 1.50 (d, J=2 Hz, 6H, 2×CH₃); MS (ESI+): [C₂₃H₃₀N₆O+H]⁺ calc. 407.25, found 407.45.

The predicted properties of compounds 22 and 32-40 are summarised in Table 3. The properties were predicted using Schrödinger software (Schrödinger Inc., New York, USA).

TABLE 3
SUMMARY OF PROPERTIES OF COMPOUNDS 22 AND 32-40

Compound No.

| Property | 22 | 32 | 33 | 34 |
|---|---|---|---|---|
| MW | 363.46 | 364.45 | 364.45 | 364.45 |
| HBD | 4 | 4 | 3 | 3 |
| c log P | 2.14 | 1.53 | 1.71 | 2.08 |
| TPSA | 88.85 | 104.35 | 91.03 | 90.90 |
| % oral | 70.15 | 65.41 | 67.23 | 75 |

Compound No.

| Property | 35 | 36 | 37 | 38 |
|---|---|---|---|---|
| MW | 424.40 | 338.45 | 353.42 | 341.42 |
| HBD | 3 | 3 | 3 | 4 |
| c log P | 3.16 | 2.31 | 0.83 | 1.24 |
| TPSA | 97.19 | 74.27 | 109.17 | 120.42 |
| % oral | 89.75 | 78.75 | 67.41 | 67.82 |

| | Compound No. | | Control Compound |
|---|---|---|---|
| Property | 39 | 40 | Nirmatrelvir |
| MW | 392.46 | 408.55 | 499.532 |
| HBD | 4 | 3 | 2 |
| c log P | 0.77 | 2.41 | 0.34 |
| TPSA | 129.59 | 87.11 | 144.63 |
| % oral | 53.09 | 79.49 | 58.12 |

wherein MW = molecular weight, HBD = hydrogen bond donors, c log P = calculated partition coefficient (log P); TPSA = topological polar surface area; % oral = predicted oral bioavailability.

### Example 6—Effect of Compounds on Neuronal Cell Death Caused by NSP5

### Materials and Methods

Primary neurons were treated with 10 μM of compounds 22 and 32-40 (prepared according to Examples 2 and 5). NSP5 was added to primary neurons at DIV11, and the test compound was added 48 hours later. The enzyme deficient mutant, NSP5 C145A, was added in place of NSP5 in the negative control (“C145A”). Cells were treated with propidium iodide 48 hours after compound addition. Cells were washed with PBS and fixed with 4% PFA. Cells were then washed and stained with DAPI. Images of cells were taken with ZEISS Axioscan 7 microscope slide scanner (Zeiss, Oberkochen, Germany). Cell death was determined by Image J analysis, counting the death cell (red, PI) vs all cells (blue, DAPI).

### Results

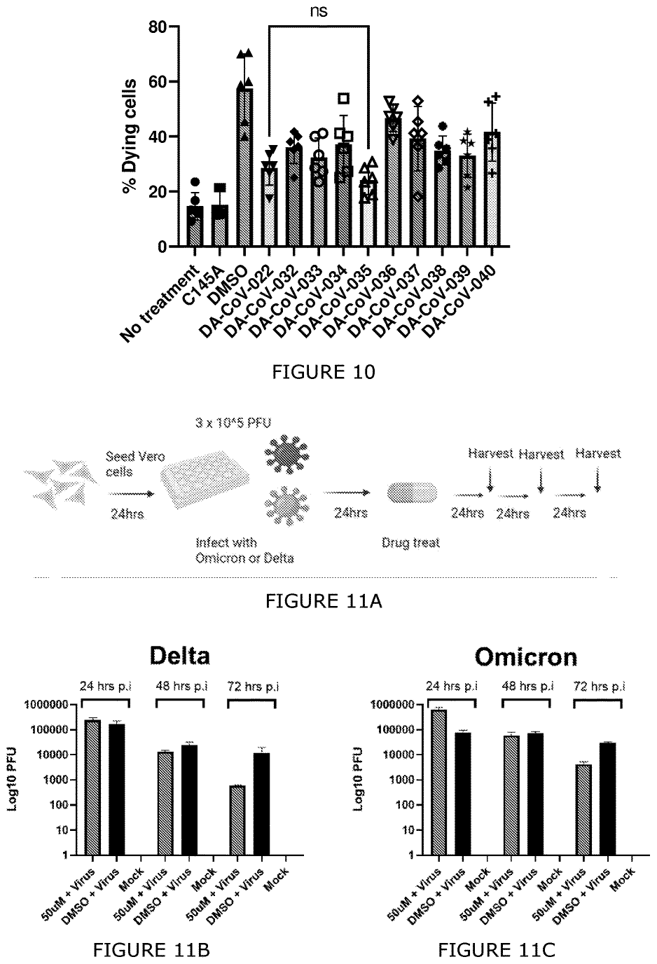

The results are presented in FIG. 10. As shown in FIG. 10, the test compounds selectively reduced neuronal cell death caused by NSP5, with compounds 22 and 35 having the greatest activity.

### Example 7—Effect of Compound 22 on Viral Replication

### Materials and Methods

Vero cells were infected with 3×10⁵ plaque-forming units (PFU) Delta or Omicron variants of SARS-CoV-2 for 24 hours before treatment with 50 μM compound 22 (prepared in accordance with Example 2). Titers of the virus were measured after 24, 48, and 72 hours incubation following treatment with compound 22. The process is outlined in FIG. 11A.

### Results

Significant reduction of titres of both Delta and Omicron variants of SARS-CoV-2 were observed after 72 hours treatment with compound 22 (refer to FIG. 11B).

### Example 8—Pharmacokinetic Properties of Compound 22

### Materials and Methods

Pharmacokinetic parameters of compound 22 (prepared in accordance with Example 2) were determined following intravenous (IV, 3 mg/kg) and intraperitoneal (IP, 10 mg/kg) administration to male C57BL/6 mice. A summary of the experimental procedures is provided in Table 4.

TABLE 4
SUMMARY OF EXPERIMENTAL PROCEDURES

| STUDY | Species | Male C57BL/6 mice | |
|---|---|---|---|
| DESIGN | Dose route | IV | IP |
| | Target Dose | 3 mg/kg | 10 mg/kg |
| | Dosing details | Bolus IV injection into the tail | Via gavage needle at a |
| | | vein using 1 mL syringe with | volume of 5 mL/kg |
| | | 25G × 1″ needle at a volume | |
| | | of 2 mL/kg | |
| | Post- dose plasma | 1, 2, 5, 15 and 30 min; 1, 2, 4, | 5, 15 and 30 min; 1, 2, 4, 7.5 |
| | collection ^{a} | 7.5 and 24 h | and 24 h |
| | Post-dose tissue ^{a} | Not required | Brain and lung were collected |
| | collection | | at 30 min, 4 and 24 h |
| FORMULATION | Vehicle | 5% (v/v) DMSO in 50 mM phosphate buffered saline (pH 6.5) | |
| | Appearance | Colourless solution | Colourless solution |
| | | (pH 6.6) | (pH 6.5) |
| | Target Concentration | 1.5 mg/mL | 2.0 mg/mL |

wherein
^{a} = plasma, brain and lung samples were also taken from two mice that were not administered compound 22 for use as analytical blanks.

### Formulation Preparation and Analysis

IV and IP formulations were prepared separately by dissolving solid compound 22 in dimethyl sulfoxide (DMSO) prior to addition of 50 mM phosphate buffered saline (pH 6.5) using vortexing and sonication to produce a colourless solution, and giving a final DMSO concentration of 5% (v/v) for each formulation. The IV formulation was filtered through a 0.22 μm syringe filter prior to dosing, whereas the IP formulation was not filtered.

The concentration of compound 22 in each formulation was determined via a suitably validated generic high performance liquid chromatography with ultraviolet spectroscopy (HPLC-UV) assay using a Waters Acquity HPLC system (Waters Corporation, Milford, USA) with a Phenomenex Ascentis Express RP-Amide column (50×2.1 mm, 2.7 μm) (Phenomenex Inc., Torrance, USA) coupled to a Waters photo diode array (PDA) detector analysing at 254 nm. The measured concentrations in both the IV and IP formulations were within 15% of the nominal concentrations, hence the nominal doses were used for data analysis.

### Mouse Pharmacokinetics

The in-life study was conducted using established procedures in accordance with the Australian Code of Practice for the Care and Use of Animals for Scientific Purposes, and the study protocols were reviewed and approved by the Monash Institute of Pharmaceutical Sciences Animal Ethics Committee.

The pharmacokinetics of compound 22 was studied in non-fasted male C57BL/6 mice weighing 21.1-26.5 g. Mice had access to food and water ad libitum throughout the pre- and post-dose sampling period.

Compound 22 was dosed to mice by bolus injection into the lateral tail vein (2 mL/kg) for IV administration and by injection into the IP cavity (5 mL/kg) for IP administration. After dosing, blood samples were collected up to 24 h (n=3 mice per time point) with a maximum of three samples from each mouse via submandibular bleed (approximately 120 μL). No urine samples were collected as mice were housed in bedded cages. Blood was collected into polypropylene Eppendorf tubes containing heparin as anticoagulant. Once collected, blood samples were centrifuged immediately, supernatant plasma was removed, and stored at −80 °C until analysis by liquid chromatography-mass spectrometry (LC-MS). A summary of the bioanalytical method is included in Table 5.

TABLE 5
SUMMARY OF BIOANALYTICAL METHOD

| Instrument | Waters Xevo TQS Micro coupled to a Waters Acquity UPLC | | | |
|---|---|---|---|---|
| Detection | Positive electrospray ionisation multiple-reaction monitoring mode | | | |
| Column | Supelco Ascentis Express RP C8 column (50 × 2.1 mm, 2.7 μm) | | | |
| LC conditions | Gradient cycle time: 4; Injection vol: 0.3 μL; Flow rate: 0.4 mL/min | | | |
| Mobile phase | (A)0.05% formic acid in water; (B) 0.05% formic acid in acetonitrile | | | |
| Sample | Plasma: Protein precipitation using acetonitrile (3-fold volume ratio) | | | |
| Preparation | Tissue: Protein precipitation using acetonitrile (3-fold volume ratio) | | | |
| Analyte | t_{R}* (min) | Transition (m/z) | Cone voltage (V) | CID^{#} (V) |
| DA-CoV-022 | 1.83 | 364.22 > 218.09 | 25 | 15 |
| Diazepam (IS) | 2.62 | 285.16 > 154.08 | 40 | 25 |

wherein IS = internal standard;
*= retention time;
^{#}= collision-induced dissociation; and DA-CoV-022 = compound 22.

For the tissue collection, at 0.5, 4 and 24 h following IP administration, mice were anaesthetised (using gaseous isoflurane). Immediately after the submandibular bleed, mice were killed via cervical dislocation and the whole brain and lung were removed from each carcase. All these tissues were blotted to remove excess blood, placed into pre-weighed polypropylene vials and weighed. Tissue samples were snap frozen in dry ice and subsequently stored frozen at −80 °C until analysis. A summary of the bioanalytical method is included in Table 5.

### In Vivo Determination of Whole Blood-to-Plasma Ratio

As part of the assessment of the IV pharmacokinetics, blood was collected from mice (n=3) at 1 h post dose for the purposes of determining the whole blood-to-plasma (B/P) ratio.

Blood was collected via cardiac puncture (whilst under gaseous isoflurane anaesthesia) into tubes containing heparin. The haematocrit was determined via centrifugation [13000×g for 4 min using a Clemets® Microhematocrit centrifuge (Clements, Rydalmere, Australia) and Safecap® Plain Self-sealing Mylar Wrapped capillary tubes (Safe-Tec Clinical Products LLC, Broomall, USA)], and values ranged from 32% to 36% for blood samples across the three mice.

Each blood aliquot (25 μL) was collected and matrix matched with an equivalent volume of blank plasma. The remainder of the blood sample was centrifuged and 1×25 μL plasma aliquot was collected and matrix matched with an equivalent volume of blank blood. Samples were frozen on dry ice and subsequently stored at −80 °C until analysis by LC-MS. The B/P ratio was calculated on the basis of the LC-MS response of compound 22 in blood and plasma following centrifugation of whole blood and the average B/P ratio (n=3 mice) is reported.

### Bioanalytical Method Summary

Concentrations of test compound in plasma and tissue samples were determined using a liquid chromatography with tandem mass spectrometry (LCMS/MS) method validated for linearity, accuracy, precision, matrix factor and recovery (refer to Table 5).

Test compound standard solutions were diluted from a concentrated stock solution (1 mg/mL in DMSO) using 50% acetonitrile in water (v/v) and a calibration curve was prepared in a matched matrix to the test samples.

Plasma: The plasma calibration curve was prepared by spiking aliquots of blank mouse plasma (25 μL) with test compound standard solutions (5 μL) and internal standard solution (5 μL of diazepam, 5 μg/mL in 50% acetonitrile in water). Test plasma samples (25 μL) were thawed, mixed and then spiked with internal standard solution (5 μL). Plasma protein precipitation was performed by addition of acetonitrile (3-fold volume ratio) and thorough vortex mixing. Samples were centrifuged (RCF=9391×g) for 3 minutes and the supernatant (100 μL) was collected for analysis. The analysis of blood/plasma (B/P) partitioning samples was conducted similarly as for the plasma samples, using protein precipitation with acetonitrile as described above.

Tissue: Pre-weighed tissue samples (brain and lung) were homogenised using a gentleMACS™ Octo Dissociator (Miltenyi Biotec, Macquarie Park, Australia) in buffer containing an EDTA/potassium fluoride solution (0.1 M/4 mg/mL) as a stabilisation cocktail to minimise the potential for ex vivo degradation (3 mL cocktail/g tissue). The tissue homogenate was briefly centrifuged (RCF=79×g) for 10 seconds to separate the foam layer before transferring an aliquot of the tissue homogenate (200 μL) to a fresh Eppendorf tube for sample extraction. Calibration standards were prepared by spiking blank brain and lung homogenate (200 μL) with the solution standards (10 μL) and the internal standard (10 μL). Study samples (for all tissue types) were similarly prepared, except that acetonitrile (10 μL) was added instead of solution standards to maintain the same volume. Protein precipitation was carried out by the addition of a 3-fold volume of acetonitrile, followed by vortex mixing and centrifugation (RCF=9391×g) for 3 min to recover the supernatant for analysis.

Replicate analysis: Triplicate analytical replicate (ARs) samples were prepared similarly to the standards for each sample type at three concentrations (50, 500 and 2000 ng/mL) and repeat injections of these ARs were included throughout the analytical run to assess assay performance. The extraction of the test compound from the standards and ARs were conducted as described above.

All test samples were quantified within the calibration range of the assay and the assay performance for ARs were acceptable. The plasma matrix factor was 128.3% and the recovery of the test compound from mouse plasma was 88.6%. The brain tissue matrix factor was 114% and the recovery of the test compound from brain tissue homogenate was 87%. The lung tissue matrix factor was 100% and the recovery of the test compound from lung tissue homogenate was 84%. The test compound was stable in brain and lung homogenates during the period of sample processing (15 min).

### Standard Calculations

The measured concentrations in both the IV and IP formulations were within 15% of the nominal concentrations, hence the nominal doses were used for data analysis.

The plasma concentration versus time profile was defined by the average plasma concentration at each sample time, and PK parameters were calculated using non-compartmental methods (PKSolver Version 2.0). Standard calculations for each pharmacokinetic parameter are listed below.

$$Plasma CL = \frac{{Dose}_{IV}}{{AUC}_{IV , 0 - inf}} Plasma CL = \frac{{AUMC}_{IV , 0 - inf}}{{AUC}_{IV , 0 - inf}} \times Plasma CL {t}_{1 / 2} = \frac{Ln ( 2 )}{{λ}_{z}}$$

$$Blood CL = \frac{Plasma CL}{B / P} Blood {V}_{ss} = \frac{Plasma {V}_{ss}}{B / P}$$

$$BA = \frac{{AUC}_{IP , 0 - last} \times {Dose}_{IV}}{{AUC}_{IV , 0 - inf} \times {Dose}_{IP}} \times 100 \% Unbound C = {C}_{total} \times {f}_{u}$$

$$CL = Clearance in plasma / blood after I V administration$$

$$AU {C}_{I V , 0 - inf} Area under the plasma concentration versus time profile from time zero to infinity after I V administration$$

$$B / P = Whole blood - to - plasma partitioning ratio$$

$${f}_{u} = Plasma unbound fraction ; a value of 0.547 was used$$

$${t}_{1 / 2} = Elimination half - life$$

$${λ}_{z} = Terminal elimination rate constant after I V administration$$

$${V}_{ss} = Apparent volume of distribution in plasma / blood at steady state$$

$${AUMC}_{IV , 0 - inf} = Area under the first movement of the plasma concentration versus time profile from time zero to infinity after I V administration$$

$$BA = I P bioavailability$$

$${AUC}_{IP , 0 - inf} = Area under the plasma concentration versus time profile from time zero to infinity after I P administration$$

$${AUC}_{0 - last} = Area under the plasma concentration v s time profile from time zero to the last time point with measureable concentration$$

$${C}_{max} = Maximum plasma concentration observed after I P administration$$

$${T}_{max} = Time to achieve {C}_{max}$$

$${C}_{total} = Total plasma concentration$$

$$Unbound C = Unbound plasma concentration$$

### Calculation of Tissue (Brain/Lung) Exposure Parameters

The concentration of compound 22 in brain or lung parenchyma was calculated on the basis of the measured concentration in brain or lung homogenate, after correcting for the contribution of compound contained within the vascular space of brain or lung samples as follows:

Cₜᵢₛₛᵤₑ=C_{tissue homogentate}−C_{tissue vasculature} where C_{tissue vasculature}=Cₚₗₐₛₘₐ×Vₚ
Cₜᵢₛₛᵤₑ=concentration of compound in brain or lung parenchyma (μM)
C_{tissue homogenate}=concentration of compound in brain or lung homogenate (μM)
C_{tissue vasculature}=concentration of compound in brain or lung vasculature (μM)
Cₚₗₐₛₘₐ=concentration of compound in plasma (μM)

Vₚ=brain plasma volume (0.017 mL/g for male C57BL/6 mice; Nicolazzo et al. (2010), Clinical and Experimental Pharmacology and Physiology, 37: 647-649) or lung plasma volume (0.16 mL/g of tissue, Boswell et al. (2014), Molecular Pharmaceutics, 11: 1591-1598)

Concentrations in brain or lung parenchyma were converted from units of ng/g to μM assuming a specific gravity of 1.

For each mouse, the tissue (brain or lung)-to-plasma concentration ratio (based on total concentrations in each matrix) was calculated as:

$$Tissue : plasma = \frac{{C}_{tissue}}{{C}_{plasma}}$$

For brain uptake, the unbound brain-to-plasma partition coefficient (based on unbound concentrations in each matrix) was calculated as:

$${K}_{p , uu} = \frac{{C}_{brain , unbound}}{{C}_{plasma , unbound}}$$

$$where$$

$${C}_{brain , unbound} = {C}_{brain} \times {f}_{u , brain} {C}_{plasma , unbound} = {C}_{plasma} \times {f}_{u , plasma}$$

$${f}_{u , brain} = unbound fraction of compound in brain tissue , using a value of {f}_{u , brain} = 0.474$$

$${f}_{u , plasma} = unbound fraction of compound in plasma , using a value of {f}_{u , plasma} = 0.547$$

### Results

The apparent whole blood-to-plasma (B/P) ratio of compound 22 in mouse blood was 1.4, indicative of effective distribution into erythrocytes.

There were no adverse reactions or compound-related side effects observed in any mice during the 24-hour sampling period after IV and IP administration of compound 22.

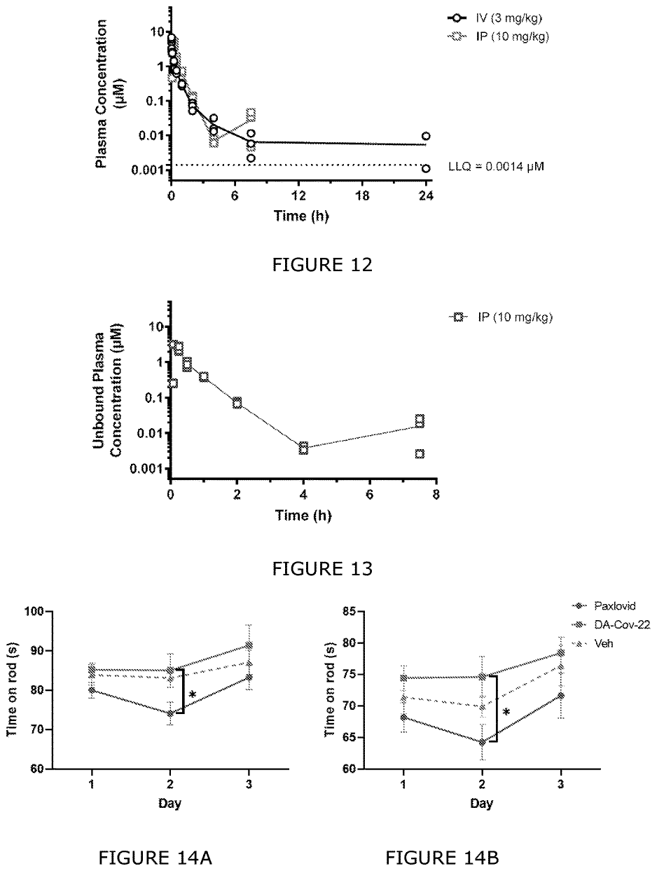

The total plasma concentration versus time profiles for compound 22 are shown in FIG. 12 and the pharmacokinetic parameters are presented in Table 6.

TABLE 6
PHARMACOKINETIC PARAMETERS FOR COMPOUND
22 IN MALE C57BL/6 MICE FOLLOWING
IV AND IP ADMINISTRATION

| | IV administration at | IP administration at |
|---|---|---|
| Parameter | 3 mg/kg | 10 mg/kg |
| Apparent t_{1/2} (h) | 14 ^{a} | c.n.c. |
| Plasma Cₘₐₓ (μM) | — | 4.25 |
| Tₘₐₓ (h) | — | 0.25 |
| Plasma AUC₀₋ₗₐₛₜ (h*μM) | 1.51 | 2.56 |
| Plasma AUC_{0-inf} (h*μM) | 1.62 ^{a} | c.n.c. |
| Plasma CL (mL/min/kg) | 84.9 ^{a} | — |
| Plasma V_{SS} (L/kg) | 23.3 ^{a} | — |
| B/P ratio | 1.4 | — |
| Blood CL (mL/min/kg) | 60.6 ^{a} | — |
| Blood V_{SS} (L/kg) | 16.6 ^{a} | — |
| BA (%) ^{b} | — | 47.3 |

wherein
^{a} = Terminal phase was not clearly defined, hence data based on extrapolation to infinity are approximations only; c.n.c. = Could not be calculated due to insufficient definition of an apparent terminal elimination phase; and
^{b} = BA calculated using the truncated AUC_{0-7.5 h}. The reported BA is therefore likely to be a slight underestimation of the actual value.

The calculated unbound plasma concentration versus time profiles of compound 22 after IP administration are shown in FIG. 13 (based on a fᵤ value of 0.547 in mouse plasma). Plasma, lung and brain concentrations at 0.5, 4 and 24 h post dose, together with the corresponding lung-to-plasma (L:P) ratios, brain-to-plasma (B:P) ratios and unbound B:P partition coefficient (K_{p,uu}) are summarised in Tables 7 and 8.

TABLE 7
PLASMA AND LUNG CONCENTRATIONS (TOTAL) AND LUNG:PLASMA
RATIOS FOR COMPOUND 22 IN MALE C57BL/6 MICE
FOLLOWING IP ADMINISTRATION AT 10 MG/KG.

| | | Total Plasma | | Total Lung Parenchyma | | | |
|---|---|---|---|---|---|---|---|
| | | Concentration (μM) | | Concentration (μM) | | L:P ratio | |
| Time | Mouse | | Mean ± | | Mean ± | | Mean ± |
| (h) | ID | Individual | SD | Individual | SD | Individual | SD |
| 0.5 | 15 | 1.92 | 1.58 ± | 3.83 | 3.80 ± | 2.0 | 2.5 ± |
| | 14 | 1.29 | 0.320 | 3.56 | 0.221 | 2.8 | 0.41 |
| | 13 | 1.54 | | 4.00 | | 2.6 | |
| 4.0 | 18 | 0.0078 | 0.0069 | 0.356 | 0.386 | 45.7 | 57.1 |
| | 17 | 0.0061 | | 0.416 | | 68.6 | |
| | 16 ^{a} | — | | — | | — | |
| 24 | 21 | ND | — | 0.0025 | 0.0021 ± | c.n.c. | — |
| | 20 | ND | | 0.0016 | 0.0009 | c.n.c. | |
| | 19 | ND | | 0.0007 | | c.n.c. | |

wherein
^{a} = Data excluded as an outlier due to a likely mis-dose in animal number 16; c.n.c. = Could not be calculated due to no measurable concentrations detected in plasma samples; and ND = not detected.

TABLE 8
PLASMA AND BRAIN CONCENTRATIONS (TOTAL AND UNBOUND),
BRAIN:PLASMA RATIOS AND UNBOUND BRAIN-TO-PLASMA PARTITION
COEFFICIENT (K_{P, UU}) FOR COMPOUND 22 IN MALE C57BL/6
MICE FOLLOWING IP ADMINISTRATION AT 10 MG/KG.

| | | Total Plasma | | Total Brain Parenchyma | | | |
|---|---|---|---|---|---|---|---|
| | | Concentration (μM) | | Concentration (μM) | | B:P ratio | |
| Time | Mouse | | Mean ± | | Mean ± | | Mean ± |
| (h) | ID | Individual | SD | Individual | SD | Individual | SD |
| 0.5 | 15 | 1.92 | 1.58 ± | 0.337 | 0.328 ± | 0.18 | 0.21 ± |
| | 14 | 1.29 | 0.320 | 0.291 | 0.0333 | 0.23 | 0.031 |
| | 13 | 1.54 | | 0.356 | | 0.23 | |
| 4.0 | 18 | 0.0078 | 0.0069 | 0.0616 | 0.0609 | 7.90 | 8.90 |
| | 17 | 0.0061 | | 0.0601 | | 9.91 | |
| | 16 ^{a} | — | | — | | — | |
| 24 | 21 | ND | — | 0.0127 | 0.0069 | c.n.c. | — |
| | 20 | ND | | 0.0011 | | c.n.c. | |
| | 19 | ND | | ND | | c.n.c. | |

| | | | | Unbound Brain | | | |
|---|---|---|---|---|---|---|---|
| | | Unbound Plasma | | Parenchyma | | Unbound B:P ratio | |
| | | Concentration (μM) | | Concentration (μM) | | (K_{p, uu}) | |
| Time | Mouse | | Mean ± | | Mean ± | | Mean ± |
| (h) | ID | Individual | SD | Individual | SD | Individual | SD |
| 0.5 | 15 | 1.05 | 0.866 ± | 0.160 | 0.155 ± | 0.15 | 0.18 ± |
| | 14 | 0.704 | 0.175 | 0.138 | 0.0158 | 0.20 | 0.027 |
| | 13 | 0.841 | | 0.169 | | 0.20 | |
| 4.0 | 18 | 0.0043 | 0.0038 | 0.0292 | 0.0288 | 6.84 | 7.72 |
| | 17 | 0.0033 | | 0.0285 | | 8.59 | |
| | 16 ^{a} | — | | — | | — | |
| 24 | 21 | — | — | 0.0060 | 0.0033 | — | — |
| | 20 | — | | 0.0005 | | — | |
| | 19 | — | | — | | — | |

wherein
^{a} = Data excluded as an outlier due to a likely mis-dose in animal number 16; c.n.c. = Could not be calculated due to no measurable concentrations detected in plasma and/or brain homogenate samples; and ND = not detected.

Following IV administration, measurable plasma concentrations were observed for the duration of the 24-hour sampling period and the profile exhibited an apparent half-life of approximately 14 h. This value should be considered as an approximation only as the terminal elimination phase was not clearly defined. The apparent blood volume of distribution was high and the blood clearance was moderate. The in vivo blood clearance corresponded to ~50% of the nominal hepatic blood flow in the mouse; assuming that systemic elimination was predominantly via hepatic clearance processes, this would suggest that compound 22 has a moderate in vivo hepatic extraction ratio.

Following IP administration, maximum plasma concentrations were observed at 0.25 h, indicative of rapid absorption. A clear terminal phase was not defined after IP administration, which precluded the determination of a half-life. The apparent bioavailability (BA) estimated on the basis of the truncated AUC was 47%.

Compound 22 exhibited measurable concentrations for up to 24 hours post dose in lung and 4 hours post dose in brain. For both tissues, the apparent tissue-to-plasma ratio (B:P or L:P) at 4 h was substantially higher than that at 0.5 h. This suggests that distributional equilibrium between each tissue and plasma was not reached by 0.5 h, and that B:P and L:P ratios determined at later time may provide a better indication of the steady state partitioning ratios for this compound.

### Example 9—In Vivo Activity of Compound 22

### Materials and Methods

In vivo testing was performed on 5-month-old female C57BL/6 wildtype mice injected intravenously with PHP.B hSyn-NSP5-V5 adeno-associated virus (AAV) and treated daily with compound 22 (10 mg/kg), Paxlovid (nirmatrelvir/ritonavir) (10 mg/kg nirmatrelvir: 3.33 mg/kg ritonavir) or a vehicle control (n=10 per group).

Motor testing was performed on the Rota-rod five times daily over a three-day testing period as previously described (refer to Ke et al. (2015) Acta Neuropathol, 130: 661-678). Motor coordination was also assessed using the hanging wire test at 14 and 22 days post injection with the AAV, which assessed the time hanging upside down on a wire grid (refer to Chan et al. (2020) Am J Path, 190(8): 1713-1722). Anxiety, disinhibition and exploratory behaviour was assessed using the elevated plus maze (EPM) test as previously described (refer to Ke et al. (2015) Acta Neuropathol, 130: 661-678).

Weight loss was monitored over a 21-day period post injection with the AAV.

### Results

Motor testing on the Rota-rod showed that compound 22 treated mice performed significantly better than Paxlovid-treated mice in both the maximum time (FIG. 14A) and the average time (FIG. 14B) of five daily trials that they were able to stay on the accelerating rotating rod over a three-day testing period.

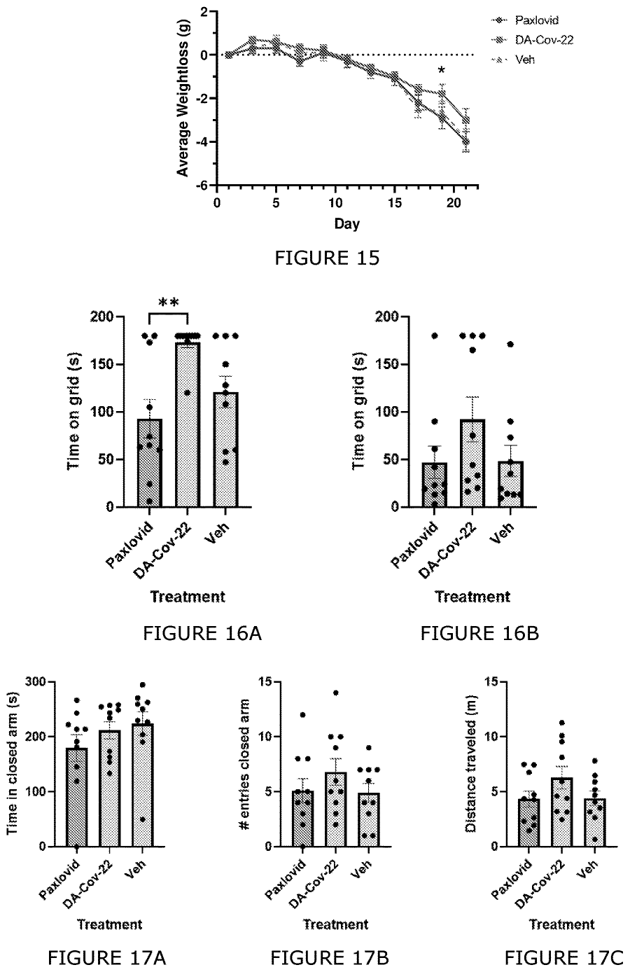

Average weight loss following AAV injection was comparable for all treatment groups until 17 days post injection, at which point weight loss was less severe in compound 22-treated mice than both Paxlovid- and vehicle-treated controls, reaching significance at day 19 and continuing until end of testing (FIG. 15).

Similarly, motor coordination testing in the hanging wire test at both early (14 days post AAV injection; FIG. 16A) and later (22 days post AAV injection; FIG. 16B) stage resulted in more compound 22-treated mice reaching the maximum time hanging upside down on the wire grid compared with Paxlovid- and vehicle-treated mice.

No significant differences were seen between the treatment groups in anxiety, disinhibition and exploratory behaviour in the elevated plus maze for total time spent in or the number of entries into the protected closed arm of the apparatus (FIGS. 17A, 17B and 17C).

The disclosure of every patent, patent application, and publication cited herein is hereby incorporated herein by reference in its entirety.

The citation of any reference herein should not be construed as an admission that such reference is available as “Prior Art” to the instant application.

Throughout the specification the aim has been to describe the preferred embodiments of the invention without limiting the invention to any one embodiment or specific collection of features. Those of skill in the art will therefore appreciate that, in light of the instant disclosure, various modifications and changes can be made in the particular embodiments exemplified without departing from the scope of the present invention. All such modifications and changes are intended to be included within the scope of the appended claims.

1. A compound of Formula I: 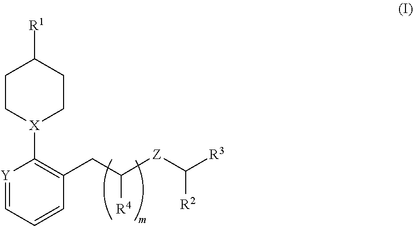
or a pharmaceutically acceptable salt thereof,
wherein:
R¹ is selected from the group consisting of H, hydroxy, optionally substituted C₁₋₆ alkyl, —NH₂, —C(O)—NH₂, optionally substituted —C(O)—C₁₋₆ alkyl, halogen, —C(OH)—NH₂, —C(O)—OH, —CH₂—NH₂, —C(O)H and optionally substituted C₁₋₆ alkoxy;
R² is selected from the group consisting of H, ═O, hydroxy, halogen, optionally substituted C₁₋₆ alkyl and optionally substituted C₁₋₆ alkoxy; and
R³ is selected from the group consisting of optionally substituted C₆₋₁₈ aryl, optionally substituted 5- to 18-membered heteroaryl, optionally substituted C₅₋₁₈ cycloalkyl, optionally substituted 5- to 18-membered heterocycloalkyl, or —C₁₋₆ alkylene-R⁵; or
R² and R³ together with the carbon atom to which they are attached form an optionally substituted C₆₋₁₈ aryl, optionally substituted 5- to 18-membered heteroaryl, optionally substituted C₅₋₁₈ cycloalkyl and optionally substituted 5- to 18-membered heterocycloalkyl;
R⁴ is selected from the group consisting of H, ═O, hydroxy, halogen, optionally substituted C₁₋₆ alkyl and optionally substituted C₁₋₆ alkoxy;
R⁵ is selected from the group consisting of optionally substituted C₆₋₁₈ aryl, optionally substituted 5- to 18-membered heteroaryl, optionally substituted C₅₋₁₈ cycloalkyl and optionally substituted 5- to 18-membered heterocycloalkyl;
m is 0 or 1;
X is selected from the group consisting of C and N;
Y is selected from the group consisting of C and N; and
Z is selected from the group consisting of C, O, N and S;
wherein the compound is other than a compound of Formula IV, V, VII, VIII, IX, XI or XII:

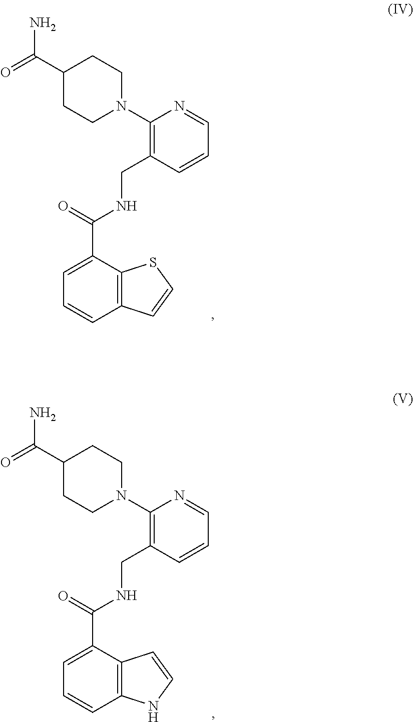 [SMILES: NC(=O)C1CCN(c2ncccc2CNC(=O)c2cccc3ccsc23)CC1 ; NC(=O)C1CCN(c2ncccc2CNC(=O)c2cccc3[nH]ccc23)CC1]

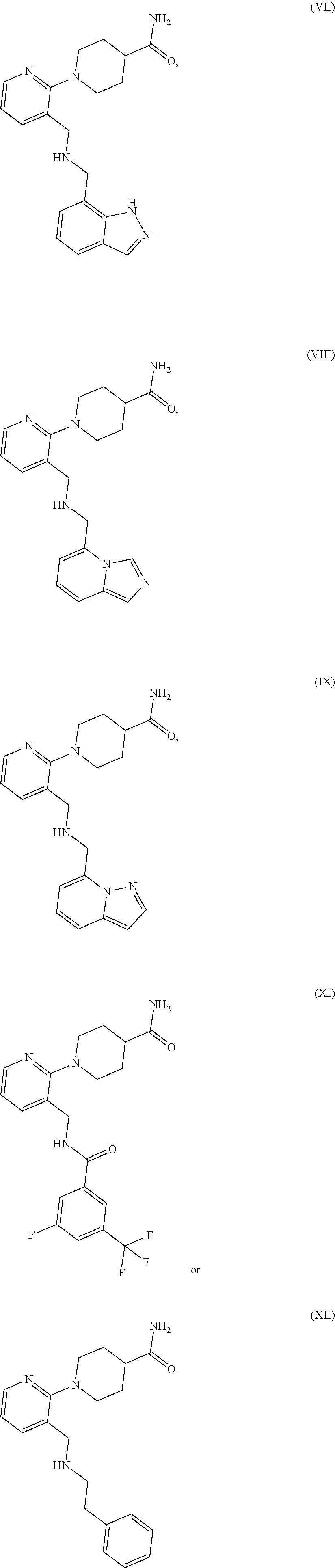 [SMILES: NC(=O)C1CCN(c2ncccc2CNCc2cccc3cn[nH]c23)CC1 ; NC(=O)C1CCN(c2ncccc2CNCc2cccc3cncn23)CC1 ; NC(=O)C1CCN(c2ncccc2CNCc2cccc3ccnn23)CC1 ; NC(=O)C1CCN(c2ncccc2CNC(=O)c2cc(F)cc(C(F)(F)F)c2)CC1 ; NC(=O)C1CCN(c2ncccc2CNCCc2ccccc2)CC1]

2. The compound according to claim 1, wherein R¹ is selected from the group consisting of —C(O)—NH₂, optionally substituted —C(O)—C₁₋₆ alkyl, —C(OH)—NH₂ and —C(O)—OH.

3. The compound according to claim 2, wherein R¹ is —C(O)—NH₂.

4. The compound according to any one of claims 1-3, wherein R² is H or ═O.

5. The compound according to any one of claims 1-4, wherein R³ is selected from the group consisting of optionally substituted C₆₋₁₈ aryl and optionally substituted 5- to 18-membered heteroaryl.

6. The compound according to claim 5, wherein R³ is optionally substituted 5- to 18-membered heteroaryl.

7. The compound according to claim 6, wherein R³ is: 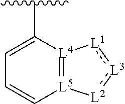
wherein L¹, L², L³, L⁴ and L⁵ are independently selected from the group consisting of C, O, N and S.

8. The compound according to claim 6, wherein R³ is: 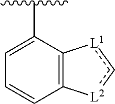
wherein:
L¹ and L² are independently selected from the group consisting of C, O, N and S.

9. The compound according to claim 8, wherein L¹ is C and L² is N or L¹ is S and L² is C.

10. The compound according to claim 8, wherein R³ is:

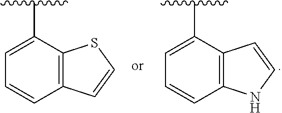 [SMILES: *c1cccc2ccsc12 ; *c1cccc2[nH]ccc12]

11. The compound according to any one of claims 1-4, wherein R³ is selected from the group consisting of: 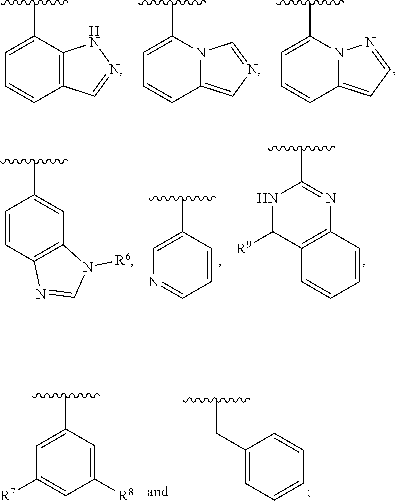
wherein R⁶ is selected from the group consisting of H, C₁₋₆ alkyl and halogen;
R⁷ is selected from the group consisting of H, C₁₋₆ alkyl, halogen and C₁₋₆ haloalkyl;
R⁸ is selected from the group consisting of H, C₁₋₆ alkyl, halogen and C₁₋₆ haloalkyl; and
R⁹ is selected from the group consisting of H, ═O, hydroxy, halogen and C₁₋₆ alkyl.

12. The compound according to claim 11, wherein R⁶ is —CH(CH₂)₂.

13. The compound according to claim 11, wherein R⁷ is —CF₃.

14. The compound according to claim 11 or claim 13, wherein R⁸ is F.

15. The compound according to claim 11, wherein R⁹ is ═O.

16. The compound according to any one of claims 1-3, wherein R² and R³ together with the carbon atom to which they are attached form an optionally substituted 5- to 18-membered heteroaryl.

17. The compound according to claim 16, wherein R² and R³ together with the carbon atom to which they are attached form: 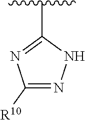
wherein R¹⁰ is selected from the group consisting of H, C₁₋₆ alkyl, C₃₋₆ cycloalkyl and halogen, and wherein 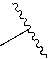
indicates the point of attachment to Z.

18. The compound according to claim 17, wherein R¹⁰ is cyclopropyl.

19. The compound according to any one of claims 1-18, wherein m is 0.

20. The compound according to any one of claims 1-18, wherein m is 1, and R⁴ is ═O.

21. The compound according to any one of claims 1-20, wherein X is N.

22. The compound according to any one of claims 1-21, wherein Y is N.

23. The compound according to any one of claims 1-22, wherein Z is C or N.

24. The compound according to claim 23, wherein Z is N.

25. The compound according to claim 1, wherein the compound is a compound of any one of Formula VI, X and XIII-XVI:

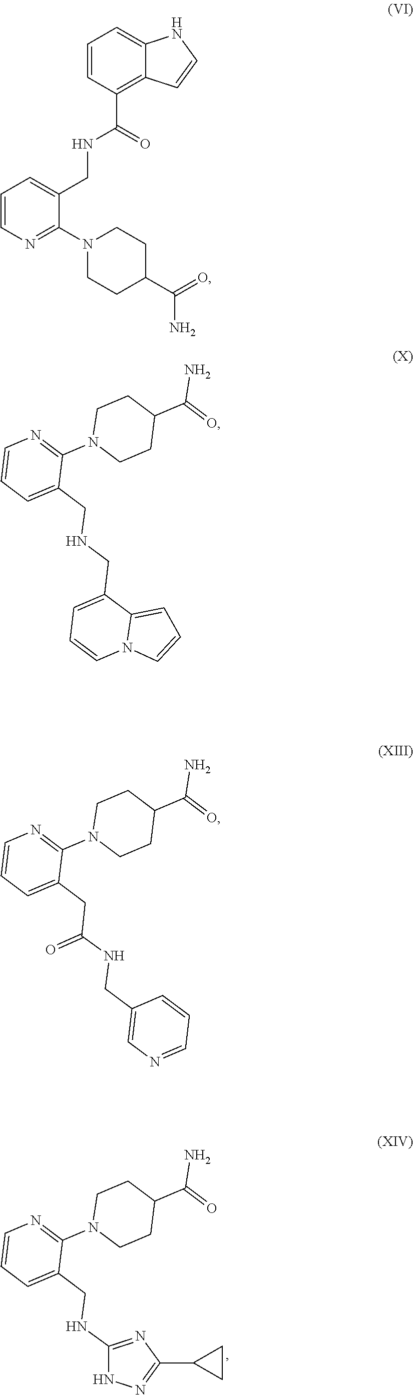

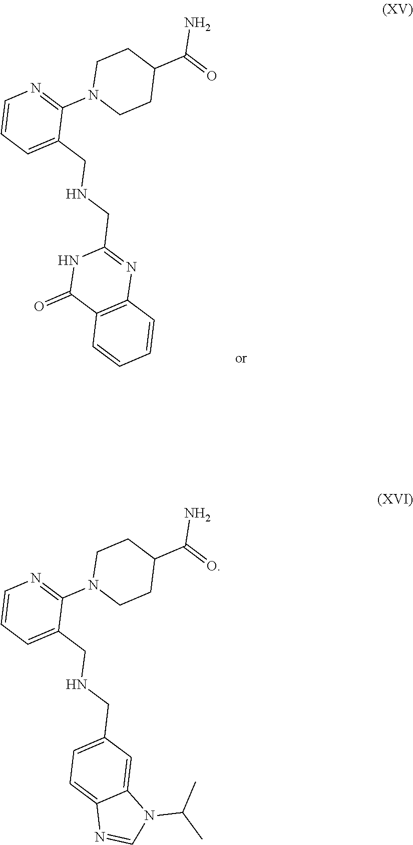 [SMILES: NC(=O)C1CCN(c2ncccc2CNCc2nc3ccccc3c(=O)[nH]2)CC1 ; CC(C)n1cnc2ccc(CNCc3cccnc3N3CCC(C(N)=O)CC3)cc21]

26. The compound according to claim 25, wherein the compound is a compound of Formula VI or XIII.

27. The compound according to claim 1, wherein the compound is a compound of Formula II or a pharmaceutically acceptable salt thereof: 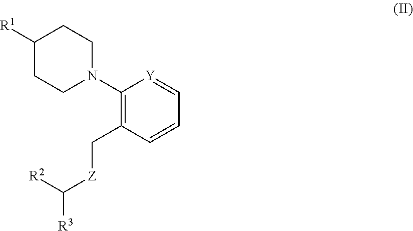
wherein:
R¹ is selected from the group consisting of H, hydroxy, optionally substituted C₁₋₆ alkyl, —NH₂, —C(O)—NH₂, optionally substituted —C(O)—C₁₋₆ alkyl, halogen, —C(OH)—NH₂, —C(O)—OH, —CH₂—NH₂, —C(O)H and optionally substituted C₁₋₆ alkoxy;
R² is selected from the group consisting of H, ═O, hydroxy, halogen, optionally substituted C₁₋₆ alkyl and optionally substituted C₁₋₆ alkoxy;
R³ is selected from the group consisting of optionally substituted C₆₋₁₈ aryl, optionally substituted 5- to 18-membered heteroaryl, optionally substituted C₅₋₁₈ cycloalkyl and optionally substituted 5- to 18-membered heterocycloalkyl;
X is selected from the group consisting of C and N;
Y is selected from the group consisting of C and N; and
Z is selected from the group consisting of C, O, N and S.

28. A composition comprising a compound or a pharmaceutically acceptable salt thereof according to any one of claims 1-27 and a pharmaceutically acceptable carrier or diluent.

29. A compound or a pharmaceutically acceptable salt thereof according to any one of claims 1-27 for use in therapy.

30. A method of inhibiting non-structural protein 5 (NSP5) activity, comprising, consisting or consisting essentially of contacting NSP5 with a compound of Formula I: 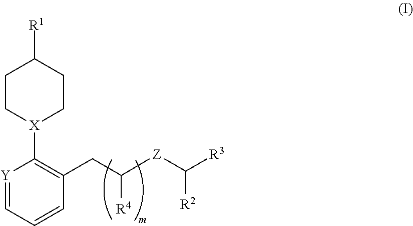
or a pharmaceutically acceptable salt thereof,
wherein:
R¹ is selected from the group consisting of H, hydroxy, optionally substituted C₁₋₆ alkyl, —NH₂, —C(O)—NH₂, optionally substituted —C(O)—C₁₋₆ alkyl, halogen, —C(OH)—NH₂, —C(O)—OH, —CH₂—NH₂, —C(O)H and optionally substituted C₁₋₆ alkoxy;
R² is selected from the group consisting of H, ═O, hydroxy, halogen, optionally substituted C₁₋₆ alkyl and optionally substituted C₁₋₆ alkoxy; and
R³ is selected from the group consisting of optionally substituted C₆₋₁₈ aryl, optionally substituted 5- to 18-membered heteroaryl, optionally substituted C₅₋₁₈ cycloalkyl, optionally substituted 5- to 18-membered heterocycloalkyl, or —C₁₋₆ alkylene-R⁵; or
R² and R³ together with the carbon atom to which they are attached form an optionally substituted C₆₋₁₈ aryl, optionally substituted 5- to 18-membered heteroaryl, optionally substituted C₅₋₁₈ cycloalkyl and optionally substituted 5- to 18-membered heterocycloalkyl;
R⁴ is selected from the group consisting of H, ═O, hydroxy, halogen, optionally substituted C₁₋₆ alkyl and optionally substituted C₁₋₆ alkoxy;
R⁵ is selected from the group consisting of optionally substituted C₆₋₁₈ aryl, optionally substituted 5- to 18-membered heteroaryl, optionally substituted C₅₋₁₈ cycloalkyl and optionally substituted 5- to 18-membered heterocycloalkyl;
m is 0 or 1;
X is selected from the group consisting of C and N;
Y is selected from the group consisting of C and N; and
Z is selected from the group consisting of C, O, N and S.

31. The method according to claim 30, wherein the NSP5 is a coronavirus NSP5.

32. The method according to claim 30 or 31, wherein the NSP5 comprises the amino acid sequence set forth in SEQ ID NO:1 or a sequence at least about 80% identical thereto.

33. A method of treating or inhibiting the development of a coronavirus infection in a subject, comprising, consisting or consisting essentially of administering a compound of Formula I or a pharmaceutically acceptable salt thereof as defined in claim 30 to the subject.

34. The method according to claim 33, wherein the coronavirus is capable of causing severe acute respiratory syndrome (SARS).

35. A method of treating or inhibiting the development of neurological symptoms of, and/or neurological complications or impairments associated with, a coronavirus infection in a subject, comprising, consisting or consisting essentially of administering a compound of Formula I or a pharmaceutically acceptable salt thereof as defined in claim 30 to the subject.

36. The method according to claim 35, wherein the one or more neurological symptoms, complications or impairments are selected from headache, fatigue, dizziness, difficulty sleeping, anxiety, depression, impaired or loss of smell (anosmia), impaired or loss of taste (dysgeusia), and cognitive challenges and/or deficits.

37. The method of claim 36, wherein the cognitive challenges and/or deficits are selected from loss of memory, impaired concentration, poor attention, confusion difficulties with thinking and difficulties with decision making.

38. The method according to any one of claims 35-37, wherein the one or more neurological symptoms, complications or impairments are experienced during the acute phase of a coronavirus infection.

39. The method according to any one of claims 35-37, wherein the one or more neurological symptoms, complications or impairments are experienced during recovery from a coronavirus infection.

40. The method according to any one of claims 35-37, wherein the one or more neurological symptoms, complications or impairments are experienced in a chronic phase following a coronavirus infection, irrespective of viral status.

41. A method for treating or inhibiting the development or progression of long COVID or one or more symptoms thereof in a subject following SARS-CoV-2 infection, comprising, consisting or consisting essentially of administering a compound of Formula I or a pharmaceutically acceptable salt thereof as defined in claim 30 to the subject.

42. The method according to claim 41, wherein one or more symptoms or manifestations of the long COVID are selected from fatigue, heart symptoms or conditions (such as myocarditis), digestive symptoms including diarrhea and stomach pain, joint or muscle pain, and one or more neurological symptoms, complications or impairments.

43. The method according to claim 42, wherein the one or more neurological symptoms, complications or impairments are selected from headache, fatigue, dizziness, difficulty sleeping, anxiety, depression, impaired or loss of smell (anosmia), impaired or loss of taste (dysgeusia), and cognitive challenges and/or deficits such as loss of memory, impaired concentration, poor attention, confusion difficulties with thinking and difficulties with decision making.

44. A method of treating an acute inflammatory condition in a subject, wherein the condition is associated with a coronavirus infection, comprising, consisting or consisting essentially of administering a compound of Formula I or a pharmaceutically acceptable salt thereof as defined in claim 30 to the subject.

45. The method according to claim 44, wherein the acute inflammatory condition is associated with the presence of cytokine release syndrome (CRS) or a cytokine storm or is associated with a multisystem inflammatory syndrome in children (MIS-C), a systemic inflammatory response syndrome (SIRS), or acute respiratory distress syndrome (ARDS).

46. The method according to claim 44 or 45, wherein the acute inflammatory condition is associated with SARS.

47. The method according to any one of claims 33-40 and 44-46, wherein the coronavirus is a betacoronavirus.

48. The method according to claim 47, wherein the betacoronavirus is SARS-CoV-2.

49. The method according to any one of claims 33-48, wherein the compound is administered orally, intranasally, by inhalation, or by infusion.

50. The method according to any one of claims 30-49, wherein R¹ is selected from the group consisting of —C(O)—NH₂, optionally substituted —C(O)—C₁₋₆ alkyl, —C(OH)—NH₂ and —C(O)—OH.

51. The method according to claim 50, wherein R¹ is —C(O)—NH₂.

52. The method according to any one of claims 30-51, wherein R² is H or ═O.

53. The method according to any one of claims 30-52, wherein R³ is selected from the group consisting of optionally substituted C₆₋₁₈ aryl and optionally substituted 5- to 18-membered heteroaryl.

54. The method according to claim 53, wherein R³ is optionally substituted 5- to 18-membered heteroaryl.

55. The method according to claim 54, wherein R³ is: 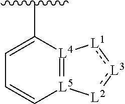
wherein L¹, L², L³, L⁴ and L⁵ are independently selected from the group consisting of C, O, N and S.

56. The method according to claim 54, wherein R³ is: 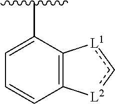
wherein:
L¹ and L² are independently selected from the group consisting of C, O, N and S.

57. The method according to claim 56, wherein L¹ is C and L² is N or L¹ is S and L² is C.

58. The method according to claim 56, wherein R³ is:

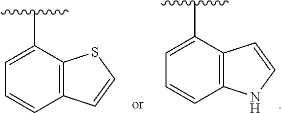 [SMILES: *c1cccc2ccsc12 ; *c1cccc2[nH]ccc12]

59. The method according to any one of claims 30-52, wherein R³ is selected from the group consisting of: 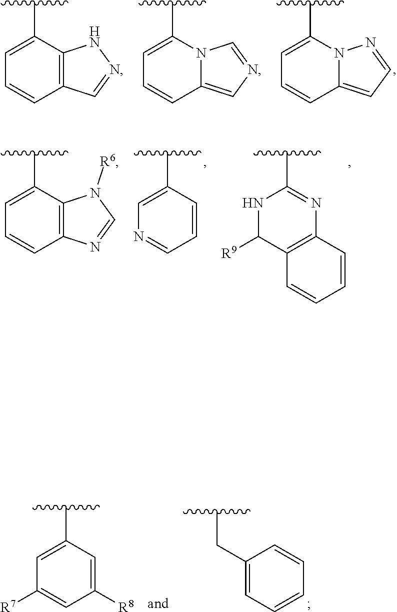
wherein R⁶ is selected from the group consisting of H, C₁₋₆ alkyl and halogen;
R⁷ is selected from the group consisting of H, C₁₋₆ alkyl, halogen and C₁₋₆ haloalkyl;
R⁸ is selected from the group consisting of H, C₁₋₆ alkyl, halogen and C₁₋₆ haloalkyl; and
R⁹ is selected from the group consisting of H, ═O, hydroxy, halogen and C₁₋₆ alkyl.

60. The method according to claim 59, wherein R⁶ is —CH(CH₂)₂.

61. The method according to claim 59, wherein R⁷ is —CF₃.

62. The method according to claim 59 or 61, wherein R⁸ is F.

63. The method according to claim 59, wherein R⁹ is ═O.

64. The method according to any one of claims 30-51, wherein R² and R³ together with the carbon atom to which they are attached form an optionally substituted 5- to 18-membered heteroaryl.

65. The method according to claim 64, wherein R² and R³ together with the carbon atom to which they are attached form: 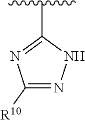
wherein R¹⁰ is selected from the group consisting of H, C₁₋₆ alkyl, C₃₋₆ cycloalkyl and halogen, and wherein 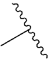
indicates the point of attachment to Z.

66. The method according to claim 65, wherein R¹⁰ is cyclopropyl.

67. The method according to any one of claims 30-66, wherein m is 0.

68. The method according to any one of claims 30-66, wherein m is 1, and R⁴ is ═O.

69. The method according to any one of claims 30-68, wherein X is N.

70. The method according to any one of claims 30-69, wherein Y is N.

71. The method according to any one of claims 30-70, wherein Z is C or N.

72. The method according to claim 71, wherein Z is N.

73. The method according to any one of claims 30-49, wherein the compound is a compound of any one of Formula IV-XVI:

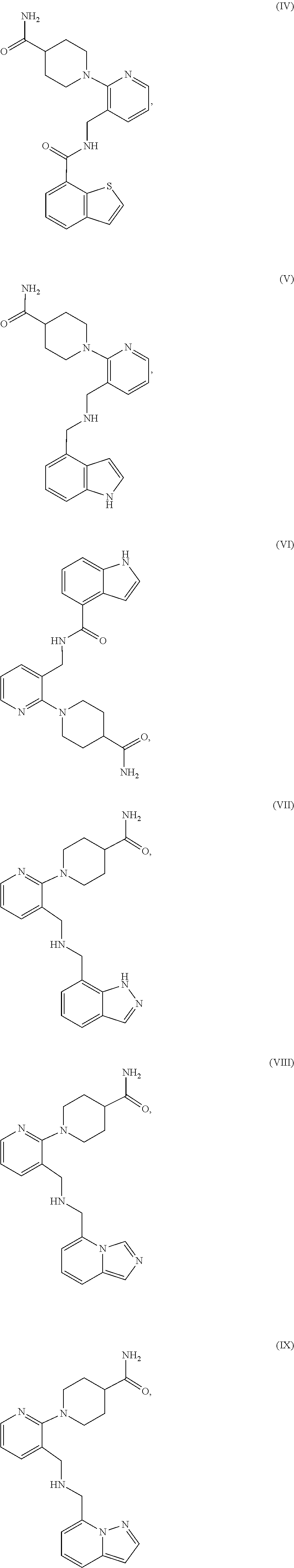 [SMILES: NC(=O)C1CCN(c2ncccc2CNC(=O)c2cccc3ccsc23)CC1 ; NC(=O)C1CCN(c2ncccc2CNCc2cccc3[nH]ccc23)CC1 ; NC(=O)C1CCN(c2ncccc2CNC(=O)c2cccc3[nH]ccc23)CC1 ; NC(=O)C1CCN(c2ncccc2CNCc2cccc3cn[nH]c23)CC1 ; NC(=O)C1CCN(c2ncccc2CNCc2cccc3cncn23)CC1 ; NC(=O)C1CCN(c2ncccc2CNCc2cccc3ccnn23)CC1]

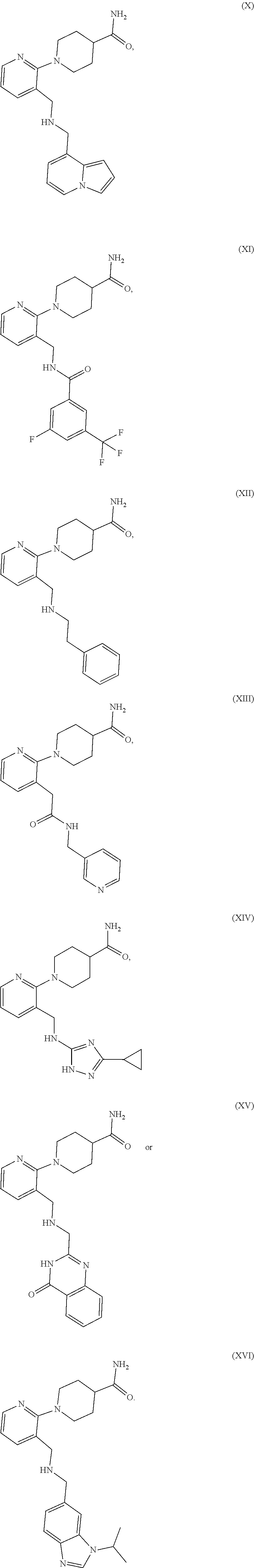

74. The method according to claim 73, wherein the compound is a compound of Formula V or XI.

75. The method according to any one of claims 30-49, wherein the compound is a compound of Formula II or a pharmaceutically acceptable salt thereof: 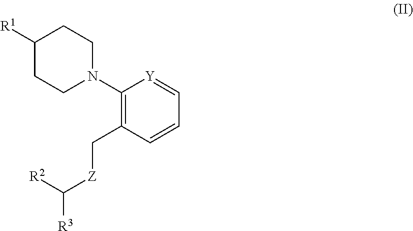
wherein:
R¹ is selected from the group consisting of H, hydroxy, optionally substituted C₁₋₆ alkyl, —NH₂, —C(O)—NH₂, optionally substituted —C(O)—C₁₋₆ alkyl, halogen, —C(OH)—NH₂, —C(O)—OH, —CH₂—NH₂, —C(O)H and optionally substituted C₁₋₆ alkoxy;
R² is selected from the group consisting of H, ═O, hydroxy, halogen, optionally substituted C₁₋₆ alkyl and optionally substituted C₁₋₆ alkoxy;
R³ is selected from the group consisting of optionally substituted C₆₋₁₈ aryl, optionally substituted 5- to 18-membered heteroaryl, optionally substituted C₅₋₁₈ cycloalkyl and optionally substituted 5- to 18-membered heterocycloalkyl;
X is selected from the group consisting of C and N;
Y is selected from the group consisting of C and N; and
Z is selected from the group consisting of C, O, N and S.
